(19) United States
(12) Patent Application Publication
(10) Pub. No.: US 2025/0263689 A1
(43) Pub. Date: Aug. 21, 2025

(54) METHODS AND SYSTEMS FOR NUCLEIC ACID ISOLATION

(71) Applicant: Seek Labs, Inc., Salt Lake City, UT (US)

(72) Inventors: Jonathan POLITIS, West Jordan, UT (US); Anindita ROY, Salt Lake City, UT (US); Madisen PYPER, Salt Lake City, UT (US); Hayden MOSCON, Salt Lake City, UT (US)

(21) Appl. No.: 19/200,382

(22) Filed: May 6, 2025

Related U.S. Application Data
(63) Continuation of application No. PCT/US2023/079525, filed on Nov. 13, 2023.
(60) Provisional application No. 63/383,426, filed on Nov. 11, 2022.
(60) Provisional application No. 63/509,861, filed on Jun. 23, 2023.

(51) Int. Cl. C12N 15/10

(52) U.S. Cl. CPC C12N 15/1017

(57) ABSTRACT

Methods, devices, and systems for isolating nucleic acid e.g., in a point of need setting, featuring a housing with a binding chamber therein, the binding chamber comprising a binding component capable of binding nucleic acid or other molecule or biomolecule of interest, and at least one fluid chamber for housing a sample or other solutions such as but not limited to buffers fluidly connected to the binding chamber. The fluids in the fluid chamber can be conveyed to and from the binding chamber, for example with the use of an actuator.

### CROSS-REFERENCES TO RELATED APPLICATIONS

This application is a continuation of International Patent Application No. PCT/US2023/079525, filed Nov. 13, 2023, which claims benefit of U.S. Provisional Application No. 63/509,861 filed Jun. 23, 2023, and U.S. Provisional Application No. 63/383,426 filed Nov. 11, 2022, the specifications of which are incorporated herein in their entirety by reference.

### FIELD OF THE INVENTION

The present invention features systems, devices, and methods for nucleic acid isolation, and includes systems, devices, and methods for isolation or filtering of other molecules, e.g., biomolecules, not limited to nucleic acid.

### BACKGROUND OF THE INVENTION

Nucleic acid detection at point-of-care is an extremely specific, sensitive, and accurate diagnostic technology that can help diagnose infectious diseases, cancer genetics, antibiotic-resistant bacteria, etc. However, sample collection and processing remain a roadblock in the current point of need for diagnostics. Specifically, sample processing methods usually require laboratory-based procedures involving multiple steps and specific equipment (e.g., pipettors, centrifuge, incubator, vortex, magnetic beads) and skilled lab personnel. This limits the use of point-of-need sample processing, especially in resource-limited settings. Currently, the gold standard has been to collect samples and send them to a testing facility where nucleic acid isolation, amplification, and detection can be carried out, and the results can take days. Most point-of-need rapid diagnostics rely on antigen-based detection methods. Nucleic acid based diagnostics are much more sensitive and accurate than antigen-based tests, with the benefit of detecting low infection levels and differentiation of pathogen variants. Thus, there is a need to develop a rapid, simple-to-use, equipment-free, cost-effective nucleic acid isolation method.

### BRIEF SUMMARY OF THE INVENTION

It is an objective of the present invention to provide systems, devices, methods, and kits that allow for the isolation of nucleic acids from samples. Non-limiting examples of types of nucleic acids isolated from samples include genomic DNA, plasmid DNA, mitochondrial RNA, ribosomal RNA, viral RNA, and other types of nucleic acids, e.g., target nucleic acids, in samples. Non-limiting examples of types of samples include biological samples, such as blood (whole blood, plasma or serum), saliva, urine, stool, bodily secretions, swabs of bodily secretions (nasal swabs, throat swabs, vaginal swabs, etc.), tissue sections or chunks from muscle, skin or other organs or glands of animals, plant parts or tissue, fungal samples, preserved biological samples, bacterial cultures, cell cultures, tissue cultures. Sample types can also be environmental samples, such as wastewater, tapwater, rainwater, samples from natural or artificial water bodies, animal waste from farms or natural environments, ground coverings in farms or natural environments. Embodiments of the present invention can be freely combined with each other if they are not mutually exclusive. It is also an objective of the present invention to provide systems, devices, methods, and kits that allow for the isolation or filtration of a particular molecule, e.g., biomolecule, other than nucleic acid.

Briefly, the present invention features a device for isolating nucleic acids from a sample. The device may comprise a housing comprising a binding chamber with a binding component capable of binding nucleic acid therein, a first chamber, and a second chamber. The device may further comprise a first channel fluidly connecting the binding chamber and the first chamber and a second channel fluidly connecting the binding chamber and the second chamber. In some embodiments, the device comprises a means for conveying a fluid through either one or both of the first channel or the second channel. For example, the device may comprise a means for conveying a first fluid through the first channel between the first chamber and the binding chamber and a means for conveying a second fluid through the second channel between the second chamber and the binding chamber. In some embodiments, the means for conveying the first fluid through the first channel comprises a first actuator operatively connected to the first chamber. In some embodiments, the means for conveying the second fluid through the second channel comprises a second actuator operatively connected to the second chamber. Alternatively, the means for conveying a first fluid through the first channel comprises a flexible vessel, tube, or vial (or other appropriate component) operatively connected to the first chamber, and the means for conveying the second fluid through the second channel comprises a flexible vessel, tube, or vial (or other appropriate component) operatively connected to the second chamber.

The device for isolating nucleic acids (or other molecules, e.g., biomolecules) from a sample may comprise a housing with a binding chamber with a binding component such as one capable of binding nucleic acid therein, a first chamber, a second chamber, a first channel, a second channel, a first actuator, and a second actuator disposed or integrated therein. The first channel fluidly connects the binding chamber and the first chamber, and the second channel fluidly connects the binding chamber and the second chamber. In some embodiments, the first actuator is operatively connected to the first chamber and configured to affect fluid flow through the first channel between the first chamber and the binding chamber. In some embodiments, the second actuator is operatively connected to the second chamber and configured to affect fluid flow through the second channel between the second chamber and the binding chamber. The housing may further comprise a first coupler fluidly connected to the first chamber and a second coupler fluidly connected to the second chamber. The first and second couplers provide an opening to the first and second chambers, respectively. In some embodiments, the first coupler and the second coupler are configured to engage a closing means, e.g., a means for closing the opening to the first or second chamber (respectively), for preventing access to the first chamber through the first coupler, for preventing access to the second chamber through the second coupler. Non-limiting examples of closing means include a cap, a seal, a plug, a film, valve, or a vial. In some embodiments, the couplers can have design elements that allow or help in the connection between the chambers in a specific direction, such as screw threads, luer locking mechanism, etc.

In some embodiments, the binding of the nucleic acid is temporary. In some embodiments, the binding of nucleic acid is strong enough to allow for one or more wash steps prior to elution. In some embodiments, the binding of nucleic acid is such that the nucleic acid can be directly used in amplification methods, e.g., the binding component interacts with amplification solutions, etc. In some embodiments, the binding component comprises cellulose. In certain embodiments, the binding component is immobilized in the binding chamber.

The present invention also features kits with one or more systems and/or devices and/or components of the systems described herein. In some embodiments, the kits comprise a lysis buffer for use with the systems and devices. Lysis buffers are well known to be an ordinary skill in the art. A lysis buffer may comprise, for example, a solution that comprises a buffering agent and a salt (or a combination of salts). In some embodiments, the lysis buffer comprises a chelating agent. In some embodiments, the lysis buffer comprises a chaotropic or denaturing agent. In some embodiments, the lysis buffer comprises one or more detergents. In some embodiments, the lysis buffer comprises one or more enzymes such as lysozyme, proteinase, RNase, DNase, or other appropriate enzyme, or a combination of enzymes. In some embodiments, the lysis buffer further comprises one or more salts. In some embodiments, the lysis buffer further comprises a chelating agent. Commercially available lysis buffers are also within the scope of the present invention. In some embodiments, the systems and devices herein do not depend on the pH of the lysis buffer. Examples of lysis buffers are further described herein. In some embodiments, the kits further comprise a wash buffer or solution for use with the systems and devices. In some embodiments, the kits further comprise an elution buffer or solution for use with the systems and devices. Wash buffers and elution buffers are well known to be an ordinary skill in the art. In some embodiments the elution solution can just be nuclease-free water. In some embodiments the wash or elution solution or buffer may comprise, for example, a solution that comprises a buffering agent. In some embodiments, the wash or elution buffer further comprises one or more salts. In some embodiments, the elution buffer further comprises a chelating agent. Commercially available wash buffers and elution buffers are also within the scope of the present invention. In some embodiments, the systems and devices herein do not depend on the pH of the wash or elution buffer. In some embodiments, the specific pH of the wash buffer or elution buffer can help enhance the performance of the systems and devices.

Although the disclosure herein describes specific embodiments and configurations of the systems and devices for isolating nucleic acid, one of ordinary skill in the art would recognize that changes or alternative configurations of the systems and devices can still teach an equivalent and effective nucleic acid isolation system and said changes and alternative configurations fall within the scope of the present invention.

Furthermore, the present invention broadly encompasses nucleic acid isolation systems (and more generally molecule, e.g., biomolecule, isolation systems) and devices featuring a spatially enclosed binding component, e.g., cellulose, glass fiber (or equivalent), and a mechanism or system for causing intentional flow of a fluid through the binding component (or binding chamber), e.g., allows for a specific direction of fluid flow.

Without wishing to limit the present invention to any theory or mechanism, it is believed that aspects of the present invention are advantageous. For example, the configuration of the spatially enclosed binding component helps prevent contamination and enhance safety since the binding component is not itself moved from one vial to the next with possible exposure to contaminants. Further, configurations of the present invention can provide for physical separation of contaminants (e.g., lysis buffer debris). For example, the systems and devices may be configured to use combinations of filtration components to physically separate contaminants from the sample instead of reagents. Additionally, the use of open channels (e.g., tubes) between chamber and/or vials described herein advantageously allows for less force to be required to move liquid around the system or device. Furthermore, the present invention provides both positive and negative pressure within the systems and devices described herein to allow for fluid to flow in a continuous circuit. None of the presently known prior references or work has the unique inventive technical feature of the present invention. As is shown in the Example, another advantage of the system is the minimal time required to perform the DNA isolation steps from start to end compared to existing systems. For example, the methods, devices, and systems can isolate DNA in under 5 minutes (e.g., 5 minutes, 4 minutes, 3 minutes, 2 minutes, 1 minute, etc.). The present invention is not limited to a maximum processing time of 5 minutes. Another advantage of the system is not necessarily requiring any equipment or machine apart from the described invention to process the sample. For example, embodiments of the methods, devices, and systems described herein can isolate DNA without requiring use of pipettes, centrifuges, incubators or any device requiring electricity or power source. Note the present invention is not limited to use without any external equipment. Yet another advantage of the system is not necessarily requiring an incubation at one specific temperature to process the sample. For example, embodiments of the methods, devices, and systems described herein can isolate DNA at room temperature or ambient temperature. Note the present invention is not limited to processing at room temperature or ambient temperature.

As used herein, “conveying a fluid” and “a means of conveying of fluid” may be used interchangeably and may refer to the process of transporting, directing, or transferring a fluid (e.g., a liquid, a gas, or any other form of fluid) from one location to another through a system and/or device. Both physical elements and the operational processes may be involved in fluid transportation. The conveying of a fluid encompasses various mechanisms and configurations, including but not limited to tubes, channels, valves, pumps, and any associated components designed to facilitate the movement of fluids. The terms encompass all known and novel approaches to fluid conveyance, as well as any modifications or improvements to existing methods or devices. In some embodiments, the systems and devices described herein may be designed for the purpose of controlling the flow, direction, or pressure of the fluid. The term “fluid” herein encompasses liquids, gasses, and mixtures thereof. For example, conveying a first fluid through the first channel refers to the transfer of the first fluid from the first chamber to the binding chamber or vice versa.

“Operational processes” refers to the specific activities, procedures, or series of steps that are carried out to make a system, device, or mechanism function or perform its intended tasks. In the context aforementioned definitions, it would include the actions and methods involved in the conveyance of fluids, such as how a pump operates, how valves control the flow of the fluid, how actuators are used to manipulate fluid direction, how the device or system is positioned, and other similar processes.

The methods, systems, and devices herein may be used for nucleic acid isolation. In some embodiments, the isolation process includes the step of extraction, e.g., introducing a solution to a sample for the purpose of freeing nucleic acid, e.g., lysing cells, solubilizing nucleic acid, etc. In some embodiments, samples introduced to the systems or devices have been treated, e.g., subjected to extraction, e.g., subjected to lysis buffer or the like. However, the terms “extraction” and “isolation” may be used interchangeably to refer to the isolative process of purifying nucleic acids.

Any feature or combination of features described herein are included within the scope of the present invention provided that the features included in any such combination are not mutually inconsistent as will be apparent from the context, this specification, and the knowledge of one of ordinary skills in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1A-1M shows various views of a three-chambered device as described herein.

FIG. 2A-2E shows a non-limiting example of how a three-chambered device, as described herein, may be used in a method of isolating nucleic acids. The present invention is not limited to the use of a sample vial (e.g., squeezable or hard) to add or collect the sample to or from the device. Other non-limiting examples of methods and devices used to add or collect the sample to or from the device include a laboratory pipette, a transfer pipette, a multichannel pipette, a syringe, or the like. Further, adding or collecting the sample may be a manual step or an automated step, e.g., carried out by an automated system such as an automated dispenser, a fraction collector, etc.

FIG. 3 shows an embodiment in which a sample (e.g., biological or environmental sample) is added to a sample vial, e.g., comprising a filter, and mechanically lysed before a lysis buffer is added to the sample vial to chemically lyse the sample.

FIG. 4A-4C shows alternative embodiments of the aforementioned three-chamber device comprising a single actuator (either a first actuator (FIG. 4A) or a second actuator (FIG. 4B)) or no actuators (FIG. 4C).

FIG. 5A and FIG. 5B show an alternative embodiment of a three-chambered device as described herein.

FIG. 6 shows one embodiment of a single-chambered device as described herein.

FIG. 7A-7F shows various embodiments of a single-chambered device described herein. FIG. 7A shows a device with no filters; FIG. 7B shows devices with one filter (e.g., an in-line filter); FIG. 7C shows a device with one filter near the binding chamber of the device; FIG. 7D shows a filter within the first coupler;

FIG. 7E shows a device with two filters (e.g., two in-line filters); and FIG. 7F shows a device with two filters, one filter being disposed in the first coupler and the other disposed near the binding chamber.

FIG. 8A-8D shows an alternative embodiment of a single-chambered device/systems described herein. The single-chambered device may comprise a binding chamber fluidly connected to either a first coupler (FIG. 8B) or a first coupler and a second coupler (FIG. 8A). The single chamber system may further comprise a binding chamber comprising a first binding chamber port and a sample chamber comprising a sample chamber port. The sample chamber port and the first binding chamber port are adapted to engage so as to fluidly connect the sample chamber and the binding chamber. The sample chamber either functions as an actuator (FIG. 8C, left) or comprises an actuator component (FIG. 8C, right). FIG. 8D shows a system further comprising a second chamber comprising a second chamber port. The second chamber port and the second binding chamber port are adapted to engage so as to fluidly connect the second chamber and the binding chamber.

FIG. 9A-9C shows various views of an adapter that may be used in conjunction with the devices of the present invention. The adapter may be used to removably attach a vial (e.g., a first vial or a second vial) to a coupler (e.g., a first coupler or a second coupler).

FIGS. 10A and 10B shows one embodiment of the systems described herein.

FIGS. 11A and 11B show various embodiments of the systems described herein.

FIG. 12A-121 shows a non-limiting example of how a system, as described herein, may be used in a method of isolating nucleic acids.

FIG. 13 shows an embodiment of the single vial system described herein.

FIG. 14A-14D shows alternative embodiments of systems described herein.

FIG. 15 shows alternative embodiments of systems described herein.

FIG. 16A-F shows the results (e.g., total gDNA yielded, the quality of DNA yielded (260/280), concentration of DNA, qPCR assay, average processing time) of samples analyzed using the system of the present invention, an alternative commercial point-of-care system, and two commercial DNA isolation kits.

### DETAILED DESCRIPTION OF THE INVENTION

Following is a list of elements corresponding to a particular element referred to herein:
100, 1100 Systems and Devices for Isolation of Nucleic Acids
110, 1110 First Vial/Sample Vial/Chamber
111, 1111 First End
112, 1112 Second End
113, 1113 Inner Cavity
114, 1114 First Sample Vial Port
115, 1115 Second Sample Vial Port
120, 1120 Binding Chamber
121, 1121 First End
122, 1122 Second End
123, 1123 Inner Cavity
124, 1124 First Binding Chamber Port
125, 1125 Second Binding Chamber Port
126, 1126 Third Binding Chamber Port
127, 1127 Fourth Binding Chamber Port
130, 1130 Second Vial/Chamber Collection/Elution Vial
131, 1131 First End
132, 1132 Second End
133, 1133 Inner cavity
134, 1134 First Vial Port
135, 1135 Second Vial Port
140a, 1140a Coupler, e.g., First Coupler
1140b Second Coupler
141, 1141 Outer Side
Inner Side 142, 1142
144, 1144 First Coupler Port
145, 1145 Second Coupler Port
146, 1146 Third Coupler Port
147, 1147 Fourth Coupler Port
149, 1149 Cap
151, 1151 First Actuator
152, 1151 Second Actuator
161, 1161 First Channel
162, 1162 Second Channel
163, 1163 Third Channel
164, 1164 Fourth Channel
165, 1165 Fifth Channel
166, 1166 Sixth Channel
170, 1170 Binding Component
180 Valve
181 First Valve
182 Second Valve
190, 1190 Filter
191, 1191 First Filter
192, 1192 Second Filter
1210 First Chamber
1211 First End
1212 Second End
1213 Inner Cavity
1220 Second Chamber
1221 First End
1222 Second End
1223 Inner Cavity

The present invention features methods, systems, and devices for isolating nucleic acid, e.g., extracting and isolating nucleic acid, from samples. The systems and devices feature the use of a housing or binding chamber with a binding component, such as one that binds nucleic acid, and mechanisms for introducing a sample, including a processed or lysed sample, to the binding component. The systems and devices also feature mechanisms for introducing an elution buffer or wash buffer to the binding component and/or obtaining the elution buffer or wash buffer. The elution buffer can be obtained from the systems and devices. Optionally, the systems and devices allow for accessing the binding component itself.

As will be described herein, the present invention also features methods, systems, and devices for isolating specific nucleic acid molecules. For example, the methods, systems, and devices herein may be modified to isolate a specific nucleic acid, e.g., the binding component may be configured to bind a specific nucleic acid.

As will be described herein, the present invention also features methods, systems, and devices for isolating molecules, e.g., biomolecules, other than nucleic acid. For example, the methods, systems, and devices may be modified to isolate a molecule, e.g., biomolecule, other than nucleic acid, e.g., the binding component may be configured to bind a molecule of interest; etc. Such methods, systems, and devices may be used for the purpose of isolation of said molecule, e.g., biomolecule, from a sample, or in some embodiments, for filtration or removal of a molecule, e.g., biomolecule, from a sample.

### Brief Description of Individual Components Described Herein

The descriptions herein pertaining to the individual components are intended solely for illustrative purposes and are not intended to impose any limitations. It is emphasized that these enumerated components are amenable to utilization in conjunction with any of the devices and systems encompassed by the present invention.

### Samples

The systems and/or devices herein may be applied to a variety of applications and optionally modified, if necessary, to be compatible with a variety of applications. In some embodiments, the sample is a tissue sample, e.g., an animal tissue sample or a plant tissue sample. In some embodiments, the sample is a cell sample. In some embodiments, the sample is a biological fluid sample, e.g., blood, urine, saliva, plasma, cerebrospinal fluid (CSF), etc. In some embodiments, the sample is a sample comprising a bacteria or other pathogen. The present invention is not limited to the aforementioned sample types. For example, the sample may comprise other biological material or cell suspensions, etc. The present invention is not limited to a particular sample size, volume, or weight. Non-limiting examples of sample weights, sizes, or volumes that the systems or devices may be configured to process include a tissue sample weighing 5 mg or less, a tissue sample weighing 100 mg or more, a blood sample having a volume of 100 uL or less, a blood sample having a volume of 1 mL or more, a plant sample cross-section size of 5 cm or more or less, etc.

In some embodiments, the sample is a liquid sample. In some embodiments, the sample comprises a combination of liquid and solid materials. In some embodiments, the sample is a biological sample. In some embodiments, the sample is an environmental sample. As used herein, the term “biological sample” includes but is not limited to a sample comprising one or more biological components such as bacteria, viruses or other pathogens, cells or tissue of a plant or other organism (e.g., fungus, insect, etc.), cells or tissue from an animal (e.g., mammalian cells or tissue), samples derived from a plant or animal or other organisms (e.g., blood, urine, etc.), etc. As used herein, the term “environmental sample” may include but is not limited to a soil sample, water sample, air sample, or other sample derived from the environment, e.g., not directly obtained from an organism.

### Binding Component

The systems, devices, and methods herein feature the use of a binding component, e.g., for binding nucleic acid, e.g., genomic DNA, target DNA, RNA, or other molecule, e.g., biomolecule. In some embodiments, the binding components are capable of reversibly binding nucleic acids. The binding components described herein may comprise any appropriate material that can bind or reversibly bind to nucleic acids (e.g., DNA, RNA). In some embodiments, the binding component comprises cellulose or cellulose-based material; however, the present invention is not limited to cellulose or a cellulose-based material. Non-limiting examples of materials that may be used as at least a part of the binding component include cellulose, nitrocellulose, modified cellulose, silica, glass fiber, a cotton pad, paper (e.g., Whatman® paper; e.g., Fusion 5 Filters), the like, or combinations thereof. In some embodiments, the binding component comprises glass fibers.

The binding component may be featured as part of a binding chamber, e.g., the binding component may be disposed inside a binding chamber. In some embodiments, the binding component is free-floating within the inner cavity of the binding chamber. In other embodiments, the binding component is immobilized in the binding chamber, e.g., the binding component may be attached to an inner wall or portion of the binding chamber. The present invention is not limited to the particular attachment sites of the binding component described herein. In some embodiments, the binding component subdivides the binding chamber into two (or more) sub-compartments or subcavities, e.g., the binding component is attached to the inner wall or cavity of the binding chamber such that the binding chamber is divided into two sub-compartments. Without wishing to limit the present invention to any theory or mechanism, it is believed that a configuration wherein the binding component subdivides the inner cavity of the binding chamber into two sub-compartments advantageously helps to keep cellular debris or tissue that may be present in the sample from contaminating the final solution, e.g., the final elution solution.

The binding component may be immobilized in the binding chamber, e.g., via an immobilizing means. Non-limiting examples of immobilizing means include a post or a combination of posts, an adhesive, a notch, a frame, a slot, a clip, or any other appropriate immobilizing means.

### Chambers

The systems, devices, and methods herein may feature the use of at least one chamber (e.g., fluid chamber, which may also be described herein as a first chamber or second chamber, binding chamber) for housing a fluid or sample, e.g., a sample from which nucleic acid is to be isolated. As described above, a sample may be a liquid sample, or a sample may comprise liquid and solid material. The term “fluid” includes a sample comprising liquid and solid material. In some embodiments, the fluid chamber houses a biological sample, environmental sample, or other appropriate sample. In some embodiments, the fluid chamber houses other fluids or solutions, such as but not limited to wash buffers, elution buffers, other buffers, or other mixtures. In some embodiments, the fluid chamber is adapted to accommodate a sample or a buffer or solution, e.g., both may be introduced to the fluid chamber at separate, appropriate times. In some embodiments, the system or device comprises two fluid chambers, wherein one fluid chamber is for housing a sample, and one fluid chamber is for housing a buffer or other solution.

The fluid chamber (or chambers) is fluidly connected to the binding chamber. In some embodiments, the fluid chamber (or chambers) is conditionally fluidly connected to the binding chamber, e.g., the flow of fluid between the fluid chamber (or chambers) and the binding chamber may be regulated in some manner to permit or prevent fluid flow at appropriate times. In some embodiments, flow of fluid between a fluid chamber and the binding chamber is permitted when the fluid chamber is engaged with the system, e.g., the fluid chamber is connected directly or indirectly to the binding chamber, and flow of fluid between said fluid chamber and the binding chamber is prevented when the fluid chamber is disengaged from the system, e.g., the chamber is disconnected (directly or indirectly) from the binding chamber, etc. In some embodiments, flow of fluid between a fluid chamber and the binding chamber is permitted when the system or device is oriented such that gravity assists the movement of fluid from the chamber to the binding chamber or from the binding chamber to the chamber. In some embodiments, flow of fluid between a fluid chamber and the binding chamber is permitted when pressure is applied to the fluid chamber, e.g., the fluid chamber is squeezed. In some embodiments, flow of fluid between a fluid chamber and the binding chamber is permitted when an external mechanism applies pressure to the fluid chamber. For example, in some embodiments, flow of fluid between a fluid chamber is permitted when an actuator is activated, the actuator being configured to direct flow of fluid from the fluid chamber to the binding chamber and/or vice versa. In some embodiments, the external mechanism comprises a squeezable tube that can directly or indirectly engage the fluid chamber and provide pressure to the fluid chamber so as to move fluid from the fluid chamber to the binding chamber. Note a squeezable tube may also function as a fluid chamber, as described herein. In some embodiments, the systems or devices comprise a valve that can help regulate the movement of fluid between the binding chamber and the fluid chamber. The present invention is not limited to the configurations for allowing, preventing, and/or regulating fluid flow described above.

Samples may be added directly to a fluid chamber and then subsequently be directed to the binding chamber. In some embodiments, samples are first added to a container, tube, vial, or other vessel (e.g., a sample vial, a collection vial, etc.), then directed to a fluid chamber, and then subsequently directed to the binding chamber. For example, as will be described herein in detail, the systems and devices may further comprise one or more containers, tubes, vials, or other vessels that can engage the system or device, e.g., directly or indirectly connected to a fluid chamber.

The devices and systems herein are shown with a single binding chamber. However, the present invention is not limited to a single binding chamber. In some embodiments, the devices or systems comprise two binding chambers, each with a binding component. In some embodiments, the devices or systems herein comprise more than two binding chambers, each with a binding component. The binding components may not necessarily all be the same in the configurations with multiple binding chambers and binding components.

### Adapter

The systems and devices herein may utilize an adapter to facilitate the attachment of a container, tube, or vial (e.g., a sample vial, a collection vial, etc.) or other vessel or component to an attachment point (e.g., coupler, port, channel, hole, valve, etc.) on the system or device, e.g., to connect directly or indirectly the container, tube, vial, or vessel to a fluid chamber. The adapter may be configured to provide a secure and leak-free connection between the container, tube, vial, or vessel and its attachment point on the device or system. In some embodiments, the adapter may comprise one or more filters (e.g., one filter, two filters, three filters, more than three filters, a plurality of filters, etc.) for filtering fluid flowing between the container, tube, vial, or vessel and the fluid chamber. In some embodiments, the filters (e.g., two filters, three filters, plurality of filters) are separated, e.g., spaced a distance apart, within the adapter. In some embodiments, the filters are evenly spaced within the adapter; however, the present invention is not limited to this configuration. As a non-limiting example, the filters may be separated by one or more spacing means, e.g., component for placing space between filters, for ensuring space remains between filters, etc. (e.g., spacers, notches, gaskets, o-rings, etc.), or the filters may be immobilized in the adapter in a configuration such that the filters are not in contact with each other or a combination thereof. In some embodiments, the filters may be arranged within the adapter in a particular manner, e.g., filters with smaller pore sizes may be arranged in a particular configuration with respect to filters with larger pore sizes. For example, filters with larger pore sizes may be positioned distally relative to the device/attachment point (e.g., coupler port, channel, hole, valve, etc.), and filters with smaller pore sizes may be positioned more proximally relative to the device/attachment point (e.g., coupler port, channel, hole, valve, etc.). The present invention is not limited to this configuration.

### Filters

The systems and devices described herein may comprise one or more filters, such as but not limited to a filter referred to herein as a prefilter or supplemental filter, e.g., a component for filtering specific cells (e.g., separating red blood cells from white blood cells, etc.), cell debris, or other materials including but not limited to a cell wall material, biological contaminants, pieces of ground tissue, the like, or a combination thereof. In some embodiments, the systems and devices comprise a single filter. In some embodiments, the systems and devices comprise two filters. In some embodiments, the systems and devices comprise three filters. In some embodiments, the systems and devices comprise four or more filters. In some embodiments, the systems and devices do not feature a filter.

In some embodiments, the filter is configured and/or positioned such that the sample passes through the filter prior to contact with the binding chamber. In some embodiments, the filter is configured and/or positioned such that the sample passes through the filter prior to contact with the binding component.

As previously discussed, a filter may be disposed in an adapter. In some embodiments, a filter may be disposed in another appropriate position in the system or device, e.g., a chamber, a channel, etc.

In some embodiments, the filter comprises a pore size selected from: 10 μm-700 μm. In some embodiments, the filter comprises a pore size selected from: 700 μm-200 μm. In other embodiments, the filter comprises a pore size selected from: 700 μm-350 μm. In further embodiments, the filter comprises a pore size selected from about 800 μm-50 μm, or about 800 μm-200 μm, or about 800 μm-350 μm, or about 800 μm-500 μm, or about 800 μm-650 μm, or about 650 μm-50 μm, or about 650 μm-200 μm, or about 650 μm-350 μm, or about 650 μm-500 μm, or about 500 μm-50 μm, or about 500 μm-200 μm, or about 500 μm-350 μm, or about 350 μm-50 μm, or about 350 μm-200 μm, or about 200 μm-50 μm. In some embodiments, the filter is removable. In other embodiments, the filter may comprise other types of filters that are porous and that do not bind to nucleic acids (e.g., DNA). As a non-limiting example, the filter, e.g., PET filter, comprises a 10 μm filter. In some embodiments, the filter comprises a 35 μm filter. In some embodiments, the filter comprises a 90 μm filter. In some embodiments, the filter comprises a 100 μm filter. In some embodiments, the filter comprises a 200 μm filter. In some embodiments, the filter comprises a 250 μm filter. In some embodiments, the filter comprises a 350 μm filter. In some embodiments, the filter comprises a 400 μm filter. In some embodiments, the filter comprises a 500 μm filter. In some embodiments, the filter comprises a 600 μm filter. In some embodiments, the filter comprises a 700 μm filter. The present invention is not limited to the aforementioned pore sizes.

In some embodiments, the filter is composed of any material that does not absorb and/or does not significantly absorb nucleic acids (e.g., absorbs or binds less than 1% of the nucleic acid in the sample, etc., e.g., absorbs or binds less than 2% of the nucleic acid in the sample, etc., e.g., absorbs or binds less than 5% of the nucleic acid in the sample, etc., e.g., absorbs or binds less than 10% of the nucleic acid in the sample, etc., absorbs or binds less than 15% of the nucleic acid in the sample, etc.). For example, the filter may comprise a polyethylene filter (PE) or a polyethylene terephthalate (PET). In some embodiments, the filter may comprise a polypropylene (PP) mesh filter. The present invention is not limited to the aforementioned filters. Accordingly, one of ordinary skill in the art would understand that different filter types may be used in accordance with the present invention depending on the sample type being processed. For example, if the sample comprises blood, leukosorb filters, transfusion filters, or the like may be used.

In some embodiments, the supplemental filter(s) may comprise a polyethylene (PET or PE) filter. In some embodiments, the supplemental filter(s) comprise a polypropylene (PP) mesh filter. In some embodiments, the supplemental filter(s) may comprise a steel mesh filter.

### Lysis Buffer

The present invention also features lysis buffers for use with the systems and devices. Lysis buffers are well known to one of ordinary skill in the art. A lysis buffer may comprise, for example, a solution that comprises a buffering agent and a salt (or a combination of salts). In some embodiments, the lysis buffer comprises a solution that comprises one or more buffering agents and one or more salts. In some embodiments, the lysis buffer further comprises a chelating agent. In some embodiments, the lysis buffer comprises a detergent. In some embodiments, the lysis buffer further comprises antifoaming agents. In some embodiments, the lysis buffer further comprises additional additives, e.g., DNA binding enhancers, chaotropic agents, enzymes (Proteinase K, Protease, Pronase, etc.), or a combination thereof. Commercially available lysis buffers are also within the scope of the present invention. In some embodiments, the systems and devices herein do not depend on the pH of the lysis buffer. As a non-limiting example, a lysis buffer may comprise Tris and NaCl. Another non-limiting example includes a lysis buffer comprising PBS.

### Three Chamber Devices and Methods of Use Thereof

The present invention features systems or devices for isolating nucleic acid, e.g., extracting and isolating nucleic acid, from a sample comprising a binding chamber with a binding component, e.g., for binding nucleic acid, disposed therein. The systems and devices may comprise two additional chambers (e.g., fluid chambers) fluidly connected to the binding chamber, one for introducing a sample to the binding chamber and one for introducing an elution buffer, a wash buffer, or other mixture to the binding chamber. Said fluid chambers can be accessed via couplers, e.g., openings in the fluid chambers. For example, when the couplers (e.g., openings) are in an open position, fluid may flow into and/or out of the fluid chambers; a sample can be introduced to a fluid chamber via the coupler (e.g., opening) when the coupler (e.g., opening) is in the open position. In some embodiments, the fluid chambers can be closed off, e.g., access to the fluid chambers can be prevented when the couplers (e.g., openings) are in the closed position. As previously discussed, the methods, systems and devices (e.g., binding component) may be modified to isolate a specific nucleic acid. The methods, systems and devices (e.g., binding component) may be modified to isolate an alternative molecule, e.g., biomolecule.

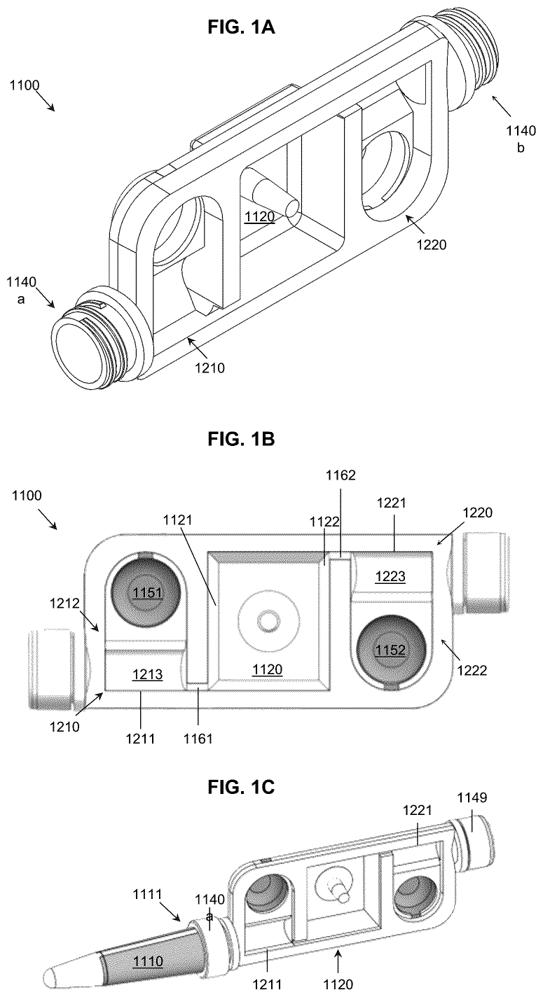

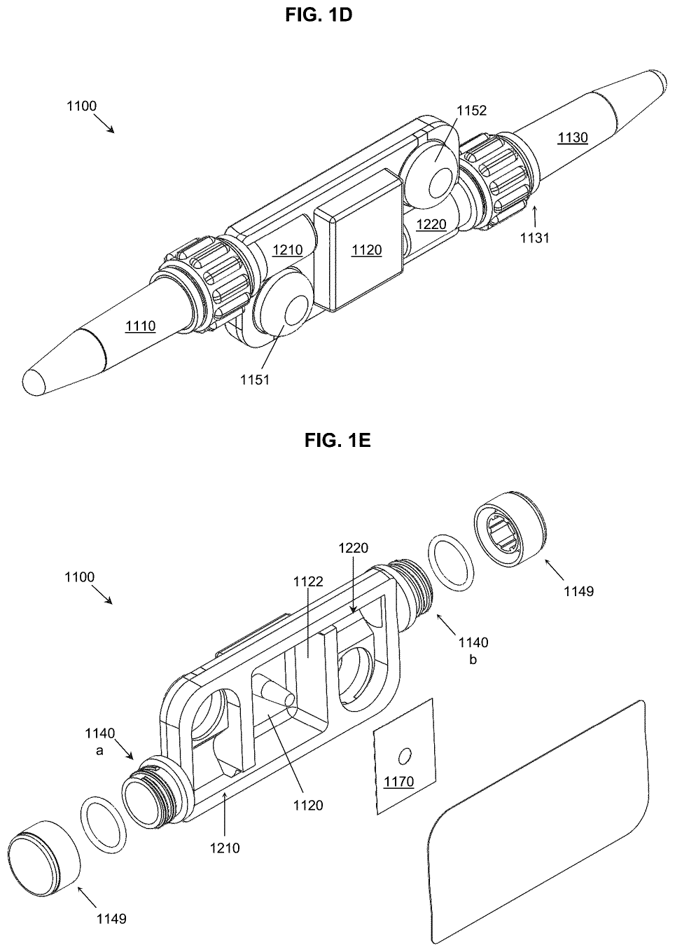

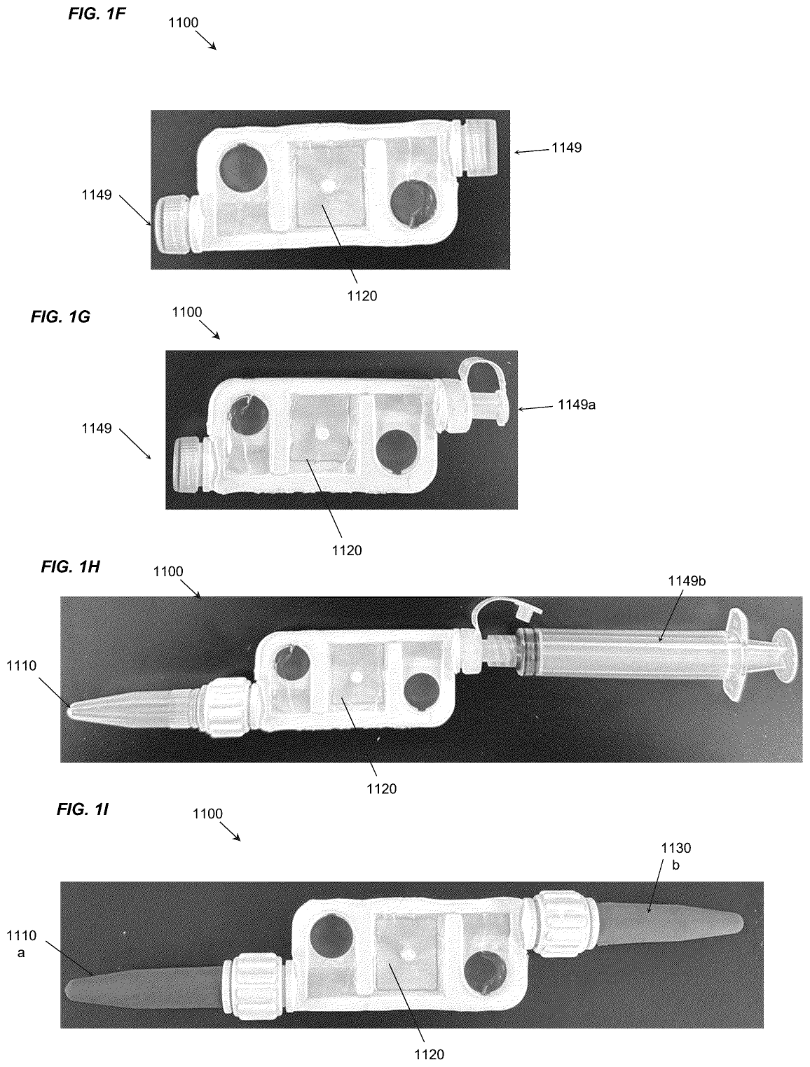

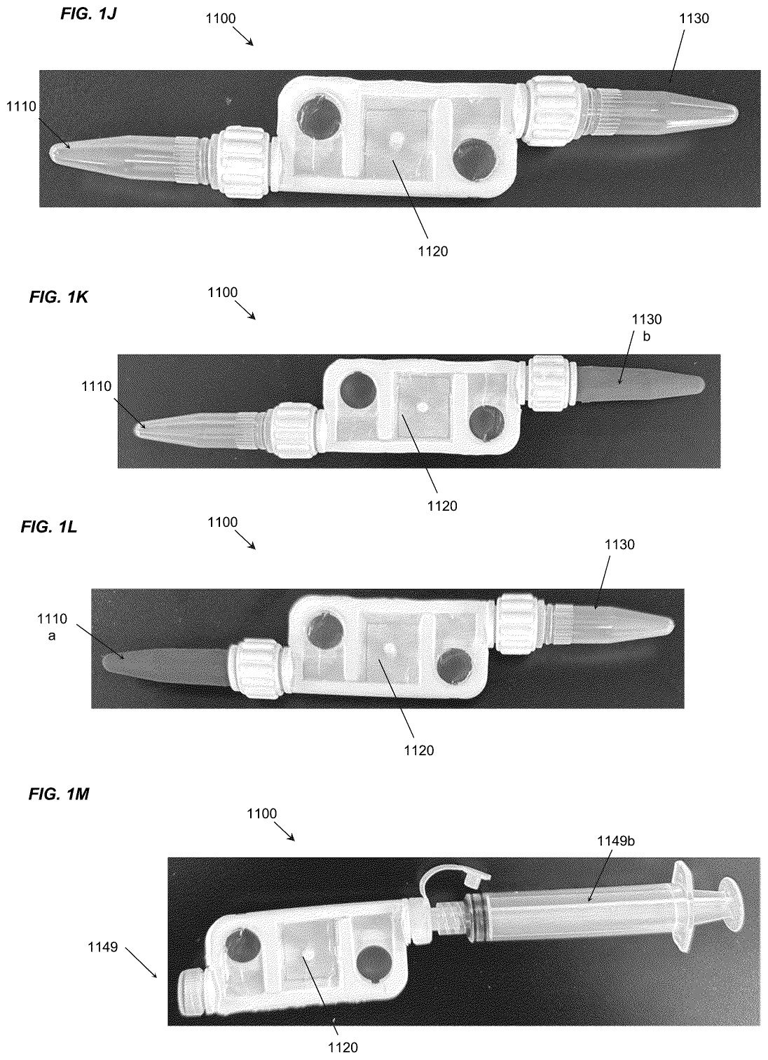

As shown in FIG. 1A-1M, the device (1100) comprises a binding chamber (1120) with a binding component (1170), e.g., capable of binding nucleic acid, disposed therein. The device (1100) further comprises a first chamber (1210) (first fluid chamber) fluidly connected to the binding chamber (1120). In some embodiments, the device (1100) further comprises a first actuator (1151) operatively and/or fluidly connected to the first chamber (1210). As will be described herein, the systems and devices may feature a variety of different actuator configurations. In some embodiments, the first actuator (1151) may be configured to affect (e.g., regulate) flow of fluid in the first chamber (1210) to and from the binding chamber (1120), e.g., assist or regulate movement of fluid. In some embodiments, the flow of fluid from the first fluid chamber to the binding chamber may not necessarily be dependent solely on the first actuator. For example, if the device comprises two actuators, a second actuator may also be used to move fluid from the first chamber to the binding chamber, e.g., either of two actuators or both actuators may be used to affect fluid flow. In some embodiments, only one actuator is used to move fluid between the first chamber and the binding chamber and the second chamber and the binding chamber.

The first chamber (1210) comprises a first coupler (1140a), which provides an opening in the first chamber (1210). The first coupler (1140a) can move between at least an open position, allowing access to the first chamber (1210), and a closed position, preventing access to the first chamber (1210). In some embodiments, the first chamber (1210) is fluidly connected to the binding chamber (1120) via a first channel (1161). For example, a first end of the first channel (1161) is fluidly connected to the binding chamber (1120), and a second end of the first channel (1161) is fluidly connected to the first chamber (1210). In some embodiments, the first actuator (1151) may be disposed opposite the first channel (1161) in the first chamber (1210). For example, the first actuator (1151) may be disposed at or near a second end (1212) of the first chamber (1210), and the first channel (1161) may be disposed at or near a first end (1211) of the first chamber (1210).

The device (1100) further comprises a second chamber (1220) (second fluid chamber) fluidly connected to the binding chamber (1120), and a second actuator (1152) operatively and/or fluidly connected to the second chamber (1220) and configured to affect (e.g., regulate) flow of fluid in the second chamber (1220) to and from the binding chamber (1120). The second chamber (1220) comprises a second coupler (1140b), which provides an opening in the second chamber (1220). The second coupler (1140b) can move between at least an open position, allowing access to the second chamber (1220), and a closed position, preventing access to the second chamber (1220). In some embodiments, the second chamber (1220) is fluidly connected to the binding chamber (1120) via a second channel (1162). For example, a first end of the second channel (1162) is fluidly connected to the binding chamber (1120), and a second end of the second channel (1162) is fluidly connected to the second chamber (1220). In some embodiments, the second actuator (1152) may be disposed opposite the second channel (1162) in the first chamber (1210). For example, the second actuator (1152) may be disposed at or near a second end (1212) of the second chamber (1220), and the second channel (1162) may be disposed at or near a first end (1211) of the second chamber (1220).

The channels, e.g., the first channel (1161), the second channel (1162), or other channels described herein, may comprise a tube or be a molded channel or may be any other appropriate conduit for fluid.

In accordance with the present invention, the binding chamber (1120) may vary in size and shape depending on the sample size and the type of sample to be processed using the devices described herein. Additionally, the binding component (1170) may vary in size and/or shape to appropriately fit into the binding chamber (1120) or to optimize binding of nucleic acid (or other molecule, e.g., biomolecule). Likewise, the binding chamber (1120) may vary in size and/or shape to appropriately fit the binding component (1170) or optimize nucleic acid (or other molecule, e.g., biomolecule) binding.

The device (1100) may be designed as a housing fabricated to contain the binding chamber (1120) and fluid chambers (e.g., first chamber (1210) and second chamber (1220)). The present invention is not limited to this configuration and includes modular systems wherein fluid chambers may be engaged to, e.g., attached to, or disengaged from, e.g., detached from, the binding chamber.

As shown in FIG. 1B, the first channel (1161) may be fluidly connected to a first side (1121) of the binding chamber (1120), and the second channel (1162) may be fluidly connected to a second side (1122) of the binding chamber. In some embodiments, the first side (1121) of the binding chamber (1120) is opposite the second side (1122) of the binding chamber (1120); however, the present invention is not limited to this configuration. In some embodiments, the first channel (1161) may be fluidly connected to a first position of the first side (1121) of the binding chamber (1120), and the second channel (1162) may be fluidly connected to a second position of the second side (1122) of the binding chamber, wherein the first position of the first side (1121) is opposite that of the second position of the second side (1122), e.g., when viewed from the side, the first position is at or near the bottom of the binding chamber and the second position is at or near the top of the binding chamber, or the first position is at or near the top of the binding chamber and the second position is at or near the bottom of the binding chamber. In some embodiments, device or system is configured (e.g., the channels described herein are positioned and/or configured) such that fluid is able to flow between the binding chamber and the first chamber but not between the binding chamber and the second chamber at the same time, or such that fluid is able to flow between the binding chamber and the second chamber but not between the binding chamber and the first chamber at the same time. As used herein, the term “exclusive” position may refer to a position or the positions of the channels wherein fluid can flow between the binding chamber and the first chamber but not the second chamber at the same time, or wherein the fluid can flow between the binding chamber and the second chamber but not the first chamber at the same time. The ability of the system or device to exclude fluid flow between the binding chamber and one of the first or second chambers while allowing fluid to flow between the binding chamber and the other of the first or second chambers may be a function one or a combination features of the system or device or other external operations or configurations, e.g., the positioning of the channels (e.g., an exclusive position), the amount of force applied to convey the fluid through one of the channels, the size of the channels, the size of the binding chamber, the size of the first or second chambers, the internal pressure, the orientation in which the device or system is held, etc.

The first coupler (1140a) and/or the second coupler (1140b) may be configured to engage a closing means, e.g., a means for moving the couplers to the closed position, a means for preventing access to the chamber. In some embodiments, the closing means comprises a cap (1149). For example, a cap (1149) may be removably attachable to the couplers so as to move the couplers to the closed position. Non-limiting examples of other closing means include a plug, a film, a wax, a seal, a cap, a valve, a tube, a vial, a flexible vial, etc.

The first coupler (1140a) may be configured to engage a first vial (1110), e.g., a sample vial, wherein fluid from the first vial may flow to the first chamber (1210). As shown in FIG. 1C, the first end (1111) of the first vial (1110) is removably attachable to the first coupler (1140a), e.g., an outer side of the first coupler (1140a). The first vial (1110) may house a sample, such as a sample in a lysis buffer. The first vial (1110) may comprise a flexible vial (1110a) or tube.

The second coupler (1140b) may be configured to engage a second vial (1130), e.g., an elution vial, wherein fluid from the second vial (1130) may flow to the second chamber (1220). As shown in FIG. 1D, the first end (1131) of the second vial (1130) is removably attachable to the second coupler (1140b), e.g., an outer side of the second coupler (1140b). The second vial (1130) may house a buffer or other liquid, such as but not limited to an elution buffer or a wash buffer. The second vial (1130) may comprise a flexible vial (1130b) or tube.

The embodiments described herein may comprise one or more filters, a prefilter, a supplemental filter, etc. The filter may comprise a component for filtering specific cells (e.g., separating red blood cells from white blood cells, etc.), cell debris, or other materials including but not limited to a cell wall material, biological contaminants, pieces of ground tissue, the like, or a combination thereof. In some embodiments, the systems and devices comprise a single filter. In some embodiments, the systems and devices comprise two filters. In some embodiments, the systems and devices comprise three filters. In some embodiments, the systems and devices comprise four or more filters. In some embodiments, the systems and devices do not feature a filter. In some embodiments, the filter is configured and/or positioned such that the sample passes through the filter prior to contact with the binding chamber. In some embodiments, the filter is configured and/or positioned such that the sample passes through the filter prior to contact with the binding component. In some embodiments, one or more filters are disposed in a channel, an adapter, a chamber, etc.

Alternative configurations of the devices and systems herein are shown in FIG. 1F-1M. FIG. 1F shows a device similar to that of FIG. 1B, wherein the couplers are sealed by threaded caps (1149). FIG. 1G shows a configuration wherein a first coupler is sealed by a threaded cap (1149) and a second coupler is sealed by a snap-on cap (1149a) (e.g., a cap that snaps on or slides into the opening of the coupler). FIG. 1H shows a configuration wherein a sample tube (1110) is connected to a first coupler and a syringe (1149b) is engaged with a second coupler. FIG. 11 shows a configuration wherein a first flexible tube (1110a) is connected to a first coupler and a second flexible tube (1130b) is connected to a second coupler. FIG. 1J shows a configuration wherein a first sample tube (1110) is connected to a first coupler and a second tube (1130) is connected to a second coupler. FIG. 1K shows a configuration wherein a second tube (1110) is connected to a first coupler and a flexible tube (1130b) is connected to a second coupler. FIG. 1L shows a configuration wherein a flexible tube (1110a) is connected to a first coupler and a second tube (1130) is connected to a second coupler. FIG. 1M shows a configuration wherein a first coupler is sealed by a threaded cap (1149a) and a syringe (1149b) is engaged with a second coupler.

In some embodiments, the caps, syringes, sample tubes, flexible tubes, etc. are translucent, clear, opaque, tinted, colored, or any variation thereof.

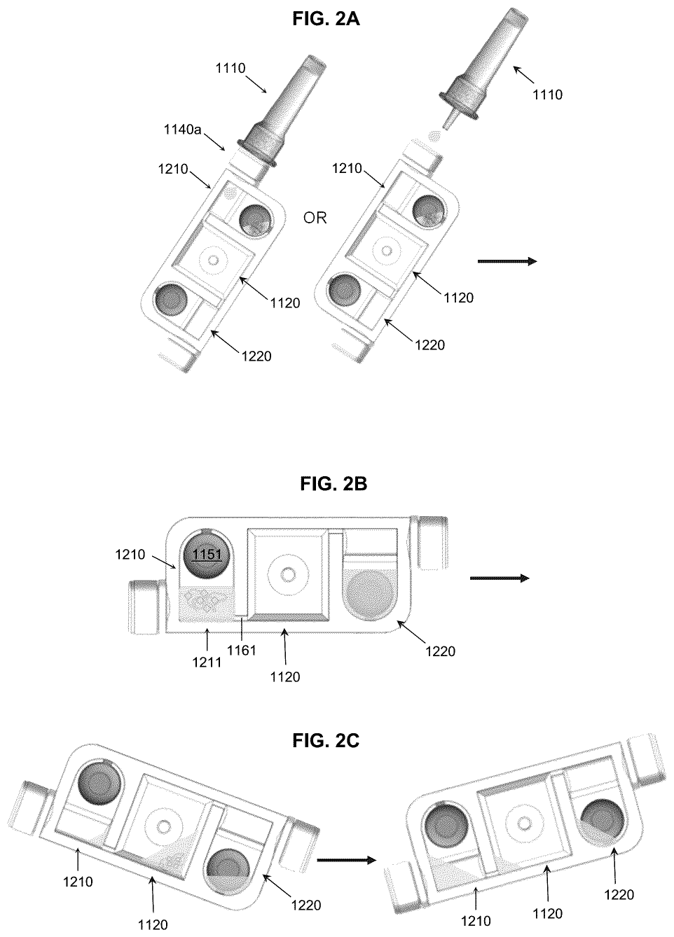

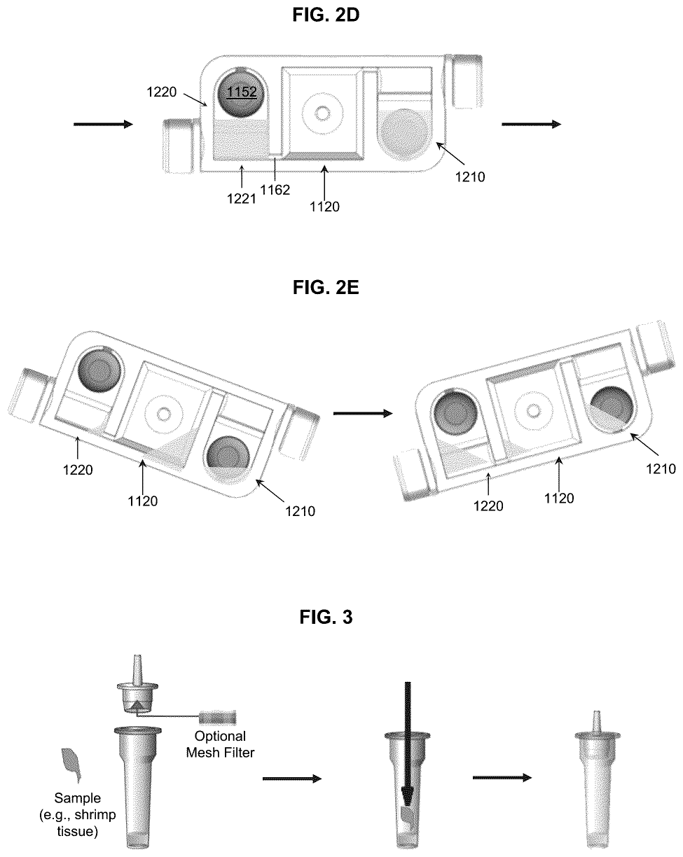

Referring to FIG. 2A-2E, the present invention also features a method of isolating nucleic acid, e.g., extracting and isolating nucleic acid, from a sample using systems and devices (1100) as described herein. The method may comprise a) adding a sample to the first chamber (1210), b) rotating the device (1100) into a first position so the sample contacts a first side (1211) of the first chamber (1210), c) pressing the first actuator (1151) at least one time to move the sample from the first chamber (1210) into the binding chamber (1120), e.g., via a first channel (1161), and d) pressing the first actuator (1151) at least one time to move the sample from the binding chamber (1120) into the first chamber (1210). As shown in FIG. 2C (left), the method may optionally comprise tilting the device (1100), e.g., at a 45° angle, to assist in moving the sample from the first chamber (1210) into the binding chamber (1120). Additionally, as shown in FIG. 2C (right), the method may optionally comprise tilting the device (1100), e.g., at a 45° angle, to assist in moving the sample from the binding chamber (1120) back into the first chamber (1210). The method may further comprise e) rotating the device (1100) into a second position so the elution buffer contacts a first side (1221) of the second chamber (1220), f) pressing the second actuator (1152) at least one time to move the sample from the second chamber (1220) into the binding chamber (1120), e.g., via a second channel (1162), and d) pressing the second actuator (1152) at least one time to move the elution buffer from the binding chamber (1120) into the second chamber (1220). As shown in FIG. 2E (left), the method may optionally comprise tilting the device (1100), e.g., at a 45° angle, to assist in moving the elution buffer from the second chamber (1220) into the binding chamber (1120). Additionally, as shown in FIG. 2E (right), the method may optionally comprise tilting the device (1100), e.g., at a 45° angle, to assist in moving the elution buffer from the binding chamber (1120) back into the second chamber (1220).

The present invention also includes the use of a particular device or system (e.g, automated, manual), configured to accept one or more of the isolation devices herein and guide or position the device at the appropriate angle, e.g., as described above.

Referring to FIG. 2A and FIG. 3, in some embodiments, the sample may be added to the first chamber (1210) by attaching a first vial (1110) to the first coupler (1140a). In other embodiments, the sample may be added to the first chamber (1210) by dispensing the sample directly into the first chamber (1210), e.g., by using a drip tube. The sample may be added to a first vial (e.g., comprising a filter) and mechanically lysed before a lysis buffer is added to the sample vial to chemically lyse the sample. The sample may then be added to the first chamber (1210) by dispensing the sample directly into the first chamber (1210), e.g., by using a drip tube. After the sample is dispensed into the first chamber (1210) a cap may be attached to the first coupler (1140a) to enclose the device. In some embodiments, the elution buffer may be pre-loaded into the device, e.g., into the second chamber (1220). In other embodiments, the elution buffer may be added into the second chamber (1220) as needed.

The present invention is not limited to the use of a sample vial (e.g., squeezable or hard) to add or collect the sample to or from the device. Other non-limiting examples of methods and devices used to add or collect the sample to or from the device include a laboratory pipette, a transfer pipette, a multichannel pipette, a syringe, or the like. Further, adding or collecting the sample may be a manual step or an automated step, e.g., carried out by an automated system such as an automated dispenser, a fraction collector, etc.

The present invention also features a method of isolating nucleic acid, e.g., extracting and isolating nucleic acid, from a sample, wherein the method may comprise using a device (1100) as described herein and a) conveying a first fluid through a first channel (1161) into a binding chamber (1120), the binding chamber (1120) comprising a binding component (1170), e.g., capable of binding nucleic acids, disposed therein, wherein the first fluid contacts the binding component (1170), b) conveying the first fluid from the binding chamber (1120) through the first channel (1161), to effectuate the removal of the first fluid from the binding chamber (1120), c) conveying a second fluid through a second channel (1162) to the binding chamber (1120), wherein the second fluid contacts the binding component (1170) and d) conveying the second fluid from the binding chamber (1120) through the second channel (1162), to effectuate the removal of the second fluid from the binding chamber (1120).

In some embodiments, the first fluid comprises a sample, e.g., a raw sample, a processed sample, a sample in a lysis buffer, a biological sample, an environmental sample, or any other appropriate sample. In some embodiments, the second fluid comprises an elution buffer. In some embodiments, the second fluid comprises a wash buffer. The present invention is not limited to the examples of first fluids and second fluids.

In some embodiments, the binding of the nucleic acid is temporary. In some embodiments, the binding of nucleic acid is strong enough to allow for one or more wash steps prior to elution. In some embodiments, the binding of nucleic acid is such that the nucleic acid can be directly used in amplification methods, e.g., the binding component interacts with amplification solutions, etc.

The devices (1100) described herein are configured such that fluid in the binding chamber (1120) necessarily contacts the binding component (1170) so that nucleic acid (or other molecule, e.g., biomolecule, of interest) in the fluid can be in contact with the binding component (1170) and/or an elution buffer can be contact with the nucleic acid (or other molecule, e.g., biomolecule, of interest) bound to the binding component (1170).

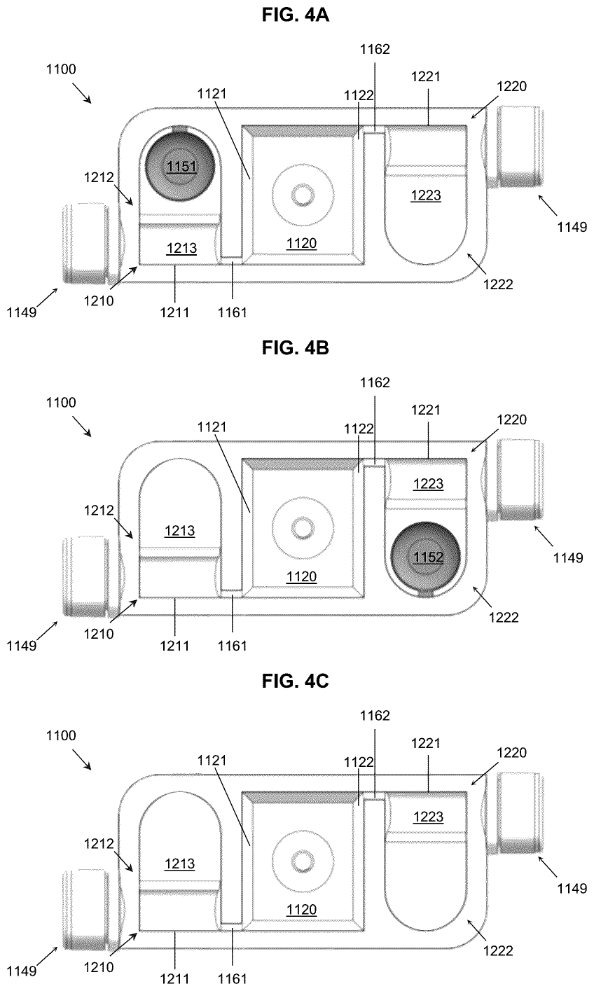

As shown in FIGS. 4A and 4B, the device (1100) may comprise a binding chamber (1120) comprising a binding component (1170), e.g., capable of binding nucleic acid, disposed therein, a first chamber (1210) fluidly connected to a first side (1121) of the binding chamber (1120), a second chamber (1220) fluidly connected to a second side (1122) of the binding chamber (1120) and either a first actuator or a second actuator. For example, as shown in FIG. 4A, the device (1100) may comprise a binding chamber (1120) comprising a binding component (1170), e.g., capable of binding nucleic acid, disposed therein, a first chamber (1210) fluidly connected to a first side (1121) of the binding chamber (1120), a second chamber (1220) fluidly connected to a second side (1122) of the binding chamber (1120) and a first actuator (1151) operatively and/or fluidly connected to the first chamber (1210). The first actuator (1151) is configured to i) affect flow of fluid in the first chamber (1210) to and from the binding chamber (1120), e.g., assist movement of fluid and/or ii) affect flow of fluid in the second chamber (1220) to and from the binding chamber (1120), e.g., assist movement of fluid. In some embodiments, the first chamber (1210) is fluidly connected to the binding chamber (1120) via a first channel (1161). For example, a first end of the first channel (1161) is fluidly connected to the binding chamber (1120), and a second end of the first channel (1161) is fluidly connected to the first chamber (1210). As previously discussed, the systems and devices (e.g., binding component) may be modified to isolate a specific nucleic acid. The systems and devices (e.g., binding component) may be modified to isolate an alternative molecule, e.g., biomolecule.

Likewise, as shown in FIG. 4B, the device (1100) may comprise a binding chamber (1120) comprising a binding component (1170), e.g., capable of binding nucleic acid, disposed therein, a first chamber (1210) fluidly connected to a first side (1121) of the binding chamber (1120), a second chamber (1220) fluidly connected to a second side (1122) of the binding chamber (1120) and a second actuator (1152) operatively and/or fluidly connected to the second chamber (1220). The second actuator (1152) is configured to i) affect flow of fluid in the first chamber (1210) to and from the binding chamber (1120), e.g., assist movement of fluid and/or ii) affect flow of fluid in the second chamber (1220) to and from the binding chamber (1120), e.g., assist movement of fluid. In some embodiments, the first chamber (1210) is fluidly connected to the binding chamber (1120) via a first channel (1161). For example, a first end of the first channel (1161) is fluidly connected to the binding chamber (1120), and a second end of the first channel (1161) is fluidly connected to the first chamber (1210).

Referring to FIG. 4C, in certain embodiments, the device (1100) comprises a binding chamber (1120) comprising a binding component (1170), e.g., capable of binding nucleic acid, disposed therein, a first chamber (1210) fluidly connected to a first side (1121) of the binding chamber (1120), a second chamber (1220) fluidly connected to a second side (1122) of the binding chamber (1120) and an external actuator. In some embodiments, the external actuator may comprise a component external to the first chamber and/or second chamber that is configured to i) affect or regulate flow of fluid in the first chamber (1210) to and from the binding chamber (1120), e.g., assist or regulate movement of fluid and/or ii) affect or regulate flow of fluid in the second chamber (1220) to and from the binding chamber (1120), e.g., assist or regulate movement of fluid. For example, the external actuator may comprise a component that provides pressure to the first chamber and/or second chamber, respectively. A non-limiting example of a component that can provide pressure to the first chamber includes a squeezable tube or vessel, a flexible vial, or the like, attached or engaged directly or indirectly to the first coupler (1140a). A non-limiting example of a component that can provide pressure to the second chamber includes a squeezable tube or vessel, a flexible vial, or the like, attached or engaged directly or indirectly to the second coupler (1140b). Alternatively, a syringe may be used to provide pressure, e.g., a syringe secured to the first coupler (1140a) and/or the second coupler (1140b) via an adaptor.

Alternatively, in certain embodiments, the device (1100) comprises a binding chamber (1120) comprising a binding component (1170), e.g., capable of binding nucleic acid, disposed therein, a first chamber (1210) fluidly connected to a first side (1121) of the binding chamber (1120), a second chamber (1220) fluidly connected to a second side (1122) of the binding chamber (1120) and an actuator fluidly connected to the binding chamber (1120). The actuator is configured to i) affect flow of fluid in the first chamber (1210) to and from the binding chamber (1120), e.g., assist movement of fluid and/or ii) affect flow of fluid in the second chamber (1220) to and from the binding chamber (1120), e.g., assist movement of fluid. In some embodiments, the first chamber (1210) is fluidly connected to the binding chamber (1120) via a first channel (1161), and the second chamber (1220) is fluidly connected to the binding chamber (1120) via a second channel (1162). For example, a first end of the first channel (1161) is fluidly connected to the binding chamber (1120), and a second end of the first channel (1161) is fluidly connected to the first chamber (1210).

### Alternative Embodiments and Methods of Use Thereof

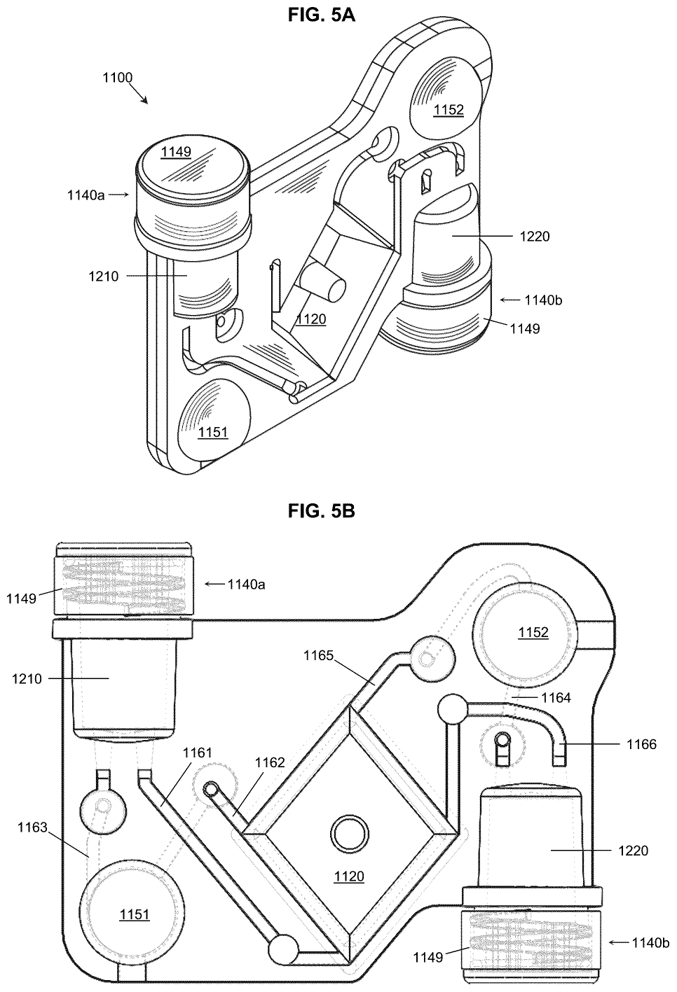

FIG. 5A and FIG. 5B shows an alternative configuration of the system or device of the present invention. The device (1100) comprises a binding chamber (1120) having an inner cavity therein, wherein a binding component, e.g., capable of binding nucleic acid, is disposed in the inner cavity. The device (1100) further comprises a first chamber (1210) fluidly connected to the binding chamber (1120) via two channels, e.g., a first channel (1161) and a second channel (1162), and a first actuator (1151) configured to assist movement of a fluid between the first chamber and binding chamber. For example, the first end of the first channel (1161) may be fluidly connected to the binding chamber (1120) and the second end configured to fluidly couple to the first chamber (1210), and the first end of the second channel (1162) may be fluidly connected to the binding chamber (1120) and the second end may be fluidly and/or operatively connected to the first actuator (1151). The device (1100) may further comprise a fifth channel (1165) having a first end fluidly connected to the binding chamber (1120), a sixth channel (1166) having a first end fluidly connected to the binding chamber (1120) and a second end configured to fluidly couple to a second chamber (1220); and a second actuator (1152) fluidly and/or operatively connected to the fifth channel (1165), wherein the second actuator (1152) is configured to assist movement of a fluid through at least the fifth channel (1165). In some embodiments, the first actuator (1151) is fluidly connected to the second end of the second channel (1162). In some embodiments, the second actuator (1152) is fluidly connected to the second end of the fifth channel (1165). In some embodiments, the first actuator (1151) is fluidly connected to a third channel (1163), and the third channel comprises an end configured to fluidly connect to the first chamber (1210). In some embodiments, the second actuator (1151) is fluidly connected to a fourth channel (1164), and the fourth channel (1164) comprises an end configured to fluidly connect to the second chamber (1220).

The present invention also features a system or device (1100) for isolating nucleic acid from a sample, wherein the device (1100) comprises a first chamber (1210) with an inner cavity for housing a fluid (e.g., sample, buffer, etc.), a first channel (1161) having a first end fluidly connected to the first chamber (1210); a binding chamber (1120) having an inner cavity (1123) therein and fluidly connected to a second end of the first channel (1161); a second channel (1162) having a second end fluidly connected to the binding chamber (1120); a third channel (1163) having a first end fluidly connected to the first chamber (1210); and a first actuator (1151), wherein the first actuator (1151) fluidly connects the second channel (1162) to the third channel (1163). When activated, the first actuator (1151) provides pressure for moving fluid, air, or both from the first chamber (1210) to the binding chamber (1120) via the first channel (1161) and from the binding chamber (1120) to the first chamber (1210) via the second channel (1162) and third channel (1163). The device (1100) further comprises a second chamber (1220) having an inner cavity for housing a fluid (e.g., buffer, wash, etc.); a fourth channel (1164) fluidly connected to the second chamber (1220); a fifth channel (1165) fluidly connected to the binding chamber (1120); a sixth channel (1165) fluidly connected to the second chamber (1220); and a second actuator (1152), wherein the second actuator (1152) fluidly connects the fourth channel (1164) to the fifth channel (1165). When activated, the second actuator (1152) provides pressure for moving fluid, air, or both from the second chamber (1220) to the binding chamber (1120) via the fourth channel (1164) and fifth channel (1165) and from the binding chamber (1120) to the second chamber (1220) via the sixth channel (1166). In some embodiments, a binding component (1170), e.g., capable of binding nucleic acid, is disposed in the inner cavity (1123) of the binding chamber (1120).

In other embodiments, the present invention features a device comprising a binding chamber (1120) comprising a binding component (1170), e.g., capable of binding nucleic acid, disposed therein; a first actuator (1151) fluidly connected to the binding chamber (1120); a first chamber (1210) fluidly connected the binding chamber (1120) and fluidly connected to the first actuator (1151); a second actuator (1152) fluidly connected to the binding chamber (1120); and a second chamber (1220) fluidly connected to the binding chamber (1120) and fluidly connected to the second actuator (1152).

In some embodiments, the first actuator (1151), the first chamber (1210), and the binding chamber (1120), as well as the second actuator (1152), the second chamber (1220), and the binding chamber (1120) are fluidly connected via a plurality of channels. A first channel (1161) fluidly connects the first chamber (1210) to the binding chamber (1120), a second channel (1162) fluidly connects the binding chamber (1120) to the first actuator (1151), and a third channel (1163) fluidly connects the first chamber (1210) to the first actuator (1151). Likewise, a fourth channel (1164) fluidly connects the second chamber (1220) to the second actuator (1152), a fifth channel (1165) fluidly connects the binding chamber (1120) to the second actuator (1152), and a sixth channel (1166) fluidly connects the binding chamber (1120) to the second chamber (1220).

As shown in FIG. 5B, the first actuator (1151) may optionally bisect a channel into a first half and a second half. The first half is the second channel (1162), and the second half is the third channel (1163). Similarly, the second actuator (1152) may optionally bisect a channel into a first half and a second half. The first half is the fourth channel (1164), and the second half is the fifth channel (1165). In some embodiments, the first actuator (1151) or second actuator (1152) is external to the channel.

In some embodiments, the first actuator (1151) or the second actuator (1152) is external to a channel. In some embodiments, the first actuator (1151) comprises a one-way valve. In some embodiments, the second actuator (1152) comprises a one-way valve. In some embodiments, the first actuator (1151), the second actuator (1152), or both are a pressure pump. In other embodiments, the second actuator (1152) comprises a one-way valve. In some embodiments, the first actuator (1151), the second actuator (1152), or both are peristaltic pumps. In some embodiments, the first actuator (1151) comprises a directional guide component. In some embodiments, the second actuator (1152) comprises a directional guide component. The directional guide component may be a valve (e.g., a one-way valve). In some embodiments, the directional guide component is a component that is adapted to allow gravity to guide fluid flow.

In some embodiments, the channels extend outwardly from the binding chamber (1120) and are arranged radially around the binding chamber (1120). In some embodiments, the channels are arranged in a configuration that occupies one or a combination of the x-axis, y-axis, and z-axis with respect to the binding chamber (1120). In some embodiments, the channels extend outwardly from the binding chamber (1120) in any particular configuration. In some embodiments, the channels are arranged radially around the binding chamber (1120) and evenly spaced.

In some embodiments, the plurality of channels comprises a plurality of tubes. In other embodiments, the plurality of channels are molded channels. The present invention is not limited to tubes and channels as a means of conveying fluid from one location to another.

The aforementioned devices may further comprise a first coupler (1140a) operably connected to the first chamber (1210) and a second coupler (1140b) operably connected to the second chamber (1220). In some embodiments, the first coupler (1140a) and the second coupler (1140b) may further comprise a cap (1149). In some embodiments, the cap (1149) is removably attached to the coupler. The cap (1149) may move between at least an open position to provide access to the first chamber (1210) or the second chamber (1220) and a closed position where the first chamber (1210) or the second chamber (1220) are not accessible. For example, a first cap (1149) may be removably attached to the first coupler (1140a) such that when the cap (1149) is removed from the first coupler (1140a), the first coupler (1140a) is in an open position and the first chamber (1210) is accessible. Additionally, when the cap (1149) is attached to the first coupler (1140a), the first coupler (1140a) is in a closed position, and the first chamber (1210) is not accessible. In some embodiments, when both the first coupler (1140a) and the second coupler (1140b) are engaged with a cap (1149), an enclosed system is created.

In accordance with the present invention, the binding chamber (1120) may vary in size and shape depending on the sample size and the type of sample to be processed using the devices described herein. Additionally, the binding component (1170) may vary in size and/or shape to appropriately fit into the binding chamber (1120) or optimize nucleic acid binding. Likewise, the binding chamber (1120) may vary in size and/or shape to appropriately fit the binding component (1170) or optimize binding of nucleic acid (or other molecule, e.g., biomolecule, of interest).

### Single Chamber Devices and Methods of Use Thereof

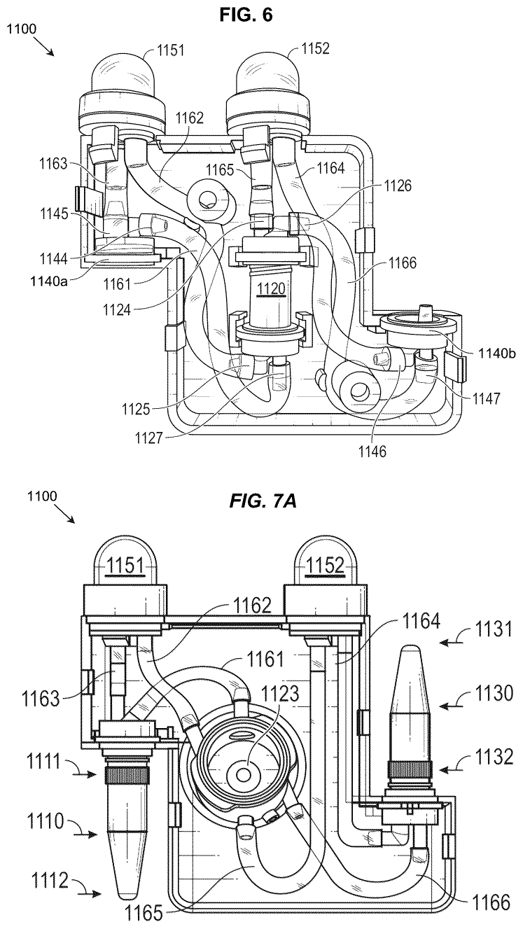

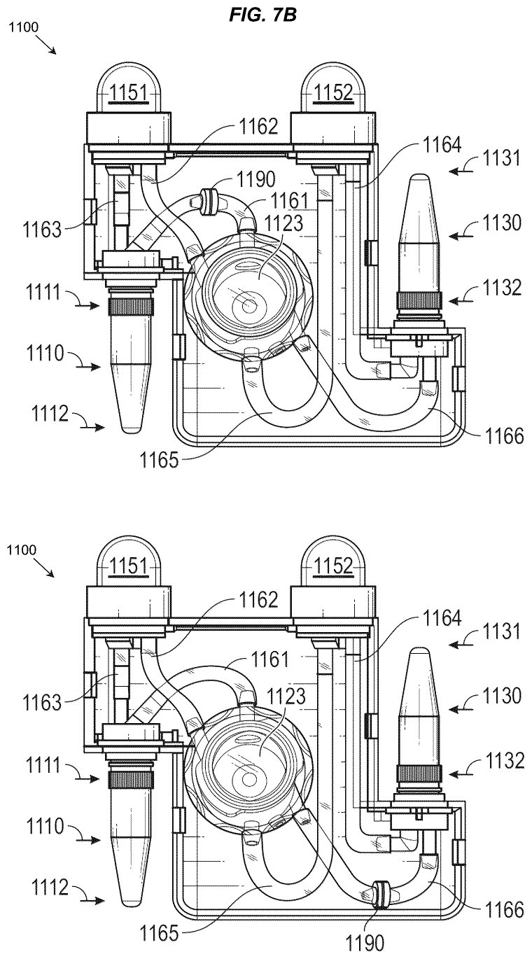

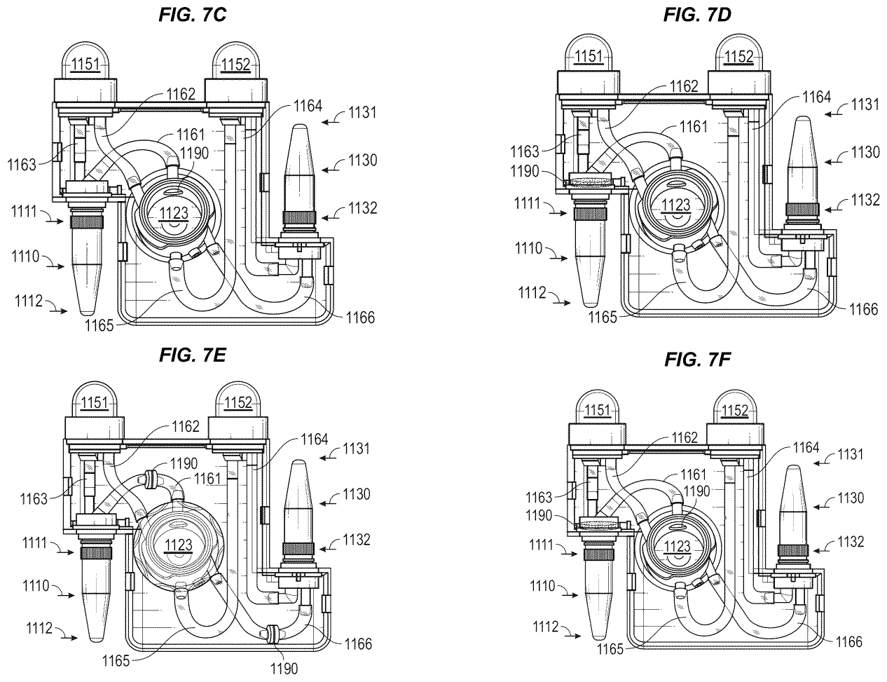

Referring to FIG. 6 and FIG. 7A-7F, the present invention may further feature a device (1100) for isolating nucleic acid from a sample. The device comprises a binding chamber having an inner cavity therein (1123), wherein a binding component (1170), e.g., capable of binding nucleic acid, is disposed in the inner cavity (1123), a first actuator (1151), a second actuator (1152), and a plurality of channels. A first channel (1161) may have a first end fluidly connected to the binding chamber (1120) and a second end configured to fluidly couple to a first vial (1110). A second channel (1162) may have a first end fluidly connected to the binding chamber (1120). In some embodiments, the first actuator (1151) may be operatively connected to the second channel (1162) and configured to assist the movement of a fluid through at least the second channel (1162). The first actuator (1151) is optionally fluidly connected to a second end of the second channel (1162). A fifth channel (1165) may have a first end fluidly connected to the binding chamber (1120), and a sixth channel (1166) may have a first end fluidly connected to the binding chamber (320) and a second end configured to fluidly couple to a second vial (1130). In some embodiments, the second actuator (1152) is operatively connected to the fifth channel (1165) and is configured to assist the movement of a fluid through at least the fifth channel (1165), the second actuator (252) is optionally fluidly connected to a second end of the fifth channel (1165).

The first actuator (1151) may be operatively connected (e.g., fluidly connected) to a third channel (1163). The third channel (1163) comprises an end configured to fluidly connect to the first vial (1110). The second actuator (1162) may be operatively connected (e.g., fluidly connected) to a fourth channel (1164). The fourth channel (1164) comprises an end configured to fluidly connect to the second vial (1130).

The present invention also features systems and devices (1100) comprising a first vial (1110) having a first end (1111) and an inner cavity (1113) for housing a sample, a binding chamber (1120) having an inner cavity (1123) therein, a first actuator (1151), a second vial (1130) having a second end (1132) and an inner cavity (1133) for housing a fluid, a second actuator (1152), and a plurality of channels. In some embodiments, the first end (1111) of the first vial (1110) comprises or is fluidly connected to a first vial port (1114) and a second vial port (1115). In some embodiments, a binding component (1170), e.g., capable of binding nucleic acid, is disposed in the inner cavity (1123) of the binding chamber (1120). In some embodiments, the binding chamber (1120) comprises or is fluidly connected to a first binding chamber port (1124), a second binding chamber port (1125), a third binding chamber port (1126), and a fourth binding chamber port (1127). A first channel may (1161) fluidly connect to the second binding chamber port (1125) and the second vial port (1115). A second channel (1162) may fluidly connect to the first binding chamber port (1124), and a third channel (1163) may fluidly connect to the first vial port (1114). The first actuator (1151) fluidly connects the second channel (1162) to the third channel (1163). In some embodiments, when activated, the first actuator (1151) provides pressure for either (i) moving fluid, air, or both from the first vial (1110) to the binding chamber (1120) via the first channel (1161) and from the binding chamber (1120) to the first vial (1110) via the second channel (1162) and third channel (1163); or (ii) moving fluid, air, or both from the first vial (1110) to the binding chamber (1120) via the second channel (1162) and third channel (1163) and from the binding chamber (1120) to the first vial (1110) via the first channel (1161). In some embodiments, the first end (1131) of the second vial (1130; e.g., a collection vial) comprises or is fluidly connected to a first vial port (1134) and a second vial port (1135). A fourth channel (1164) may fluidly connect to the first vial port (1134). A fifth channel (1165) may fluidly connect to the third binding chamber port (1126), and a sixth channel (1166) may fluidly connect to the fourth binding chamber port (1127) and the second vial port (1135). In some embodiments, the second actuator (1152) fluidly connects the fourth channel (1164) to the fifth channel (1165). In some embodiments, when activated, the second actuator (1152) provides pressure for either (i) moving fluid, air, or both from the second vial (1130) to the binding chamber (1120) via the fourth channel (1164) and fifth channel (1165) and from the binding chamber (1120) to the second vial (1130; e.g., a collection vial) via the sixth channel (1166); or (ii) moving fluid, air, or both from the second vial (1130; e.g., a collection vial) to the binding chamber (1120) via the sixth channel (1166) and from the binding chamber (1120) to the second vial (1130; e.g., a collection vial) via the fourth channel (1164) and fifth channel (1165).

The present invention may further feature a device (1100) comprising a binding chamber (1120) comprising a binding component (1170), e.g., capable of binding nucleic acid, disposed therein, a first actuator (1151) fluidly connected to the binding chamber (1120), a first coupler (1140a) fluidly connected the binding chamber (1120) and fluidly connected to the first actuator (1151), a second actuator (1152) fluidly connected to the binding chamber (1120), and a second coupler (1140b) fluidly connected to the binding chamber (1120) and fluidly connected to the second actuator (1152). In some embodiments, the first coupler and second coupler (1140b) comprise an inner side (1142) and an outer side (1141).

In some embodiments, a first end (1121) of the binding chamber (1120) comprises or is fluidly connected to a third binding chamber port (1126) and a fourth binding chamber port (1127), and a second end (1122) of the binding chamber (1120) comprises or is fluidly connected to a first binding chamber port (1124) and a second binding chamber port (1125).

The first and second couplers may comprise an inner side (1142) and an outer side (1141). The inner side (1142) of the first coupler (1140a) comprises or is fluidly connected to a first coupler port (1144) and a second coupler port (1145), and the inner side (1142) of the second coupler (1140b) comprises or is fluidly connected to a third coupler port (1146) and a fourth coupler port (1147). In some embodiments, the first coupler (1140a) comprises a filter (e.g., a prefilter) disposed therein (FIG. 7D). In other embodiments, the first coupler (1140a), the second coupler (1140b), or both the first and second coupler comprises a filter disposed therein.

In some embodiments, the first actuator (1151), the first coupler (1140a), and the binding chamber (1120), as well as the second actuator (1152), the second coupler (1140b), and the binding chamber (1120) are fluidly connected via a plurality of channels. For example, a first channel (1161) may fluidly connect the second coupler port (1145) to the second binding chamber port (1125), a second channel (1162) may fluidly connect the first binding chamber port (1124) to the first actuator (1151), and a third channel (1163) fluidly connects the first actuator (1151) to the first coupler port (1144). Likewise, a fourth channel (1164) may fluidly connect the third coupler port (1146) to the second actuator (1152), a fifth channel (1165) may fluidly connect the third binding chamber port (1126) to the second actuator (1152), and a sixth channel (1166) may fluidly connect the fourth binding chamber port 1127) to the fourth coupler port (1147). In some embodiments, the first channel (1161) may comprise a filter (1190; e.g., a first filter (1191); e.g., an inline filter) disposed therein (FIG. 7B, top). In some embodiments, the sixth channel (1166) may comprise a filter (1190; e.g., a second filter (1191); e.g., an inline filter) disposed therein (FIG. 7B, bottom). Referring to FIG. 7E, the first channel (1161) and the sixth channel (1166) may comprise a filter (1190) disposed therein (e.g., a first filter (1191) disposed within the first channel (1161) and a second filter (1192) disposed within the sixth channel (1166)).

In accordance with the present invention, the binding chamber (1120) may vary in size and shape depending on the sample size and the type of sample to be processed using the devices described herein. Additionally, the binding component (1170) may vary in size to appropriately fit into the binding chamber (1120). Likewise, the binding chamber (1120) may vary in size to appropriately fit into the binding component (1170).

In some embodiments, the binding chamber (1120) in the aforementioned devices comprises a cap. The cap may move between at least an open position to provide access to the inner cavity (1123) of the binding chamber (1120) or the binding component (1170) or both, and a closed position where the inner cavity (1123) of the binding chamber (1120) and binding component (1170) are not accessible. In some embodiments, the binding component (1170) is attached to the cap.

In some embodiments, the first actuator (1151) optionally bisects a channel into a first half and a second half, where the first half is the second channel (1162) and the second half is the third channel (1163). In some embodiments, the second actuator (1152) optionally bisects a channel into a first half and a second half, where the first half is the fourth channel (1164) and the second half is the fifth channel (1165).

In some embodiments, the plurality of channels comprises a plurality of tubes. In other embodiments, the plurality of channels are molded channels.

In some embodiments, the first actuator (1151) is fluidly connected to the binding chamber (1120) directly. In other embodiments, the first actuator (1151) is fluidly connected to the binding chamber (1120) indirectly.

The devices (300) described herein are configured such that fluid in the binding chamber (320) necessarily contacts the binding component (370).

The present invention also features a method of isolating nucleic acid from a sample, wherein the method may comprise using a device (1100) as described herein to convey a first fluid through a first channel (1161) into a binding chamber (1120) such that the first fluid contacts a binding component (1170). The binding chamber (1120) may comprise a binding component (1170), e.g., capable of binding nucleic acids, disposed therein. The first fluid is then conveyed from the binding chamber (1120) through a second channel (1162) to or towards a first actuator (1151) to effectuate the removal of the first fluid from the binding chamber (1120). Next, a second fluid is conveyed through a fifth channel (1165) to the binding chamber (1120), where the second fluid contacts the binding component (1170). Then the second fluid is conveyed from the binding chamber (1120) through a sixth channel (1162) to effectuate the removal of the second fluid from the binding chamber (1120). As previously discussed, the methods, systems and devices (e.g., binding component) may be modified to isolate a specific nucleic acid. The methods, systems and devices (e.g., binding component) may be modified to isolate an alternative molecule, e.g., biomolecule.

In some embodiments, said method may comprise using a device (1100) as described herein to convey a first fluid through a first channel (1161) into a binding chamber (1120) such that the first fluid contacts the binding component (1170). The binding chamber (1120) may comprise a binding component (1170), e.g., capable of binding nucleic acids, disposed therein. The first fluid is then conveyed from the binding chamber (1120) through a second channel (1162) to or towards a first actuator (1151) to effectuate the removal of the first fluid from the binding chamber (1120). Next, a second fluid is conveyed through a fifth channel (1165) to the binding chamber (1120), where the second fluid contacts the binding component (1170). Then the second fluid is conveyed from the binding chamber (1120) through a fifth channel (1165) to effectuate the removal of the second fluid from the binding chamber (1120).

In some embodiments, the first fluid comprises a lysis buffer, a biological sample, or a combination thereof. In other embodiments, the first fluid comprises a lysis buffer, an environmental sample, or a combination thereof. In some embodiments, the second fluid comprises an elution buffer. In other embodiments, the second fluid comprises a wash buffer.

In some embodiments, the method comprises conveying the first fluid from a first vial (1110) through the first channel (1161) to the binding chamber (1120).

In some embodiments, the method comprises conveying the first fluid from the first actuator (1151) through a third channel (1163). In other embodiments, the method comprises conveying the first fluid from the first actuator (1151) through a third channel (1163) into a first vial (1110).

In some embodiments, the method comprises conveying the second fluid from a second actuator (1152) through the fifth channel (1165). In other embodiments, the method comprises conveying the second fluid through a fourth channel (1164) to the second actuator (1152) and further conveying the second fluid from the second actuator (1152) through the fifth channel (1165) to the binding chamber (1120). In further embodiments, the method comprises conveying the second fluid from a second vial (1130) through a fourth channel (1164) to a second actuator (1152) and further conveying the second fluid from the second actuator (1152) through the fifth channel (1165) to the binding chamber (1120).

In some embodiments, the method comprises conveying the second fluid from the binding chamber (1120) through the sixth channel (1166) to a second vial (1130).

In some embodiments, the first actuator (1151) functions as a waste chamber.

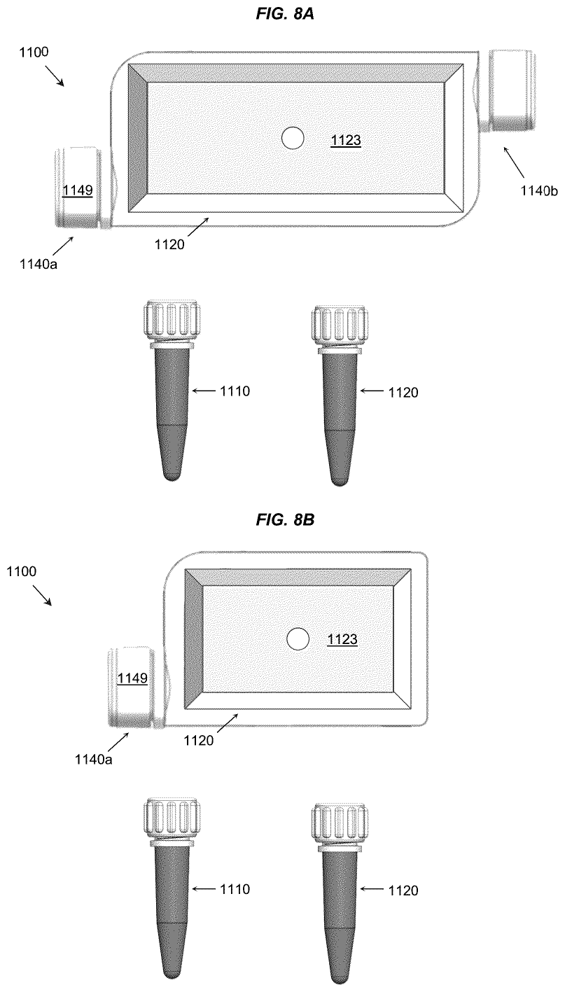

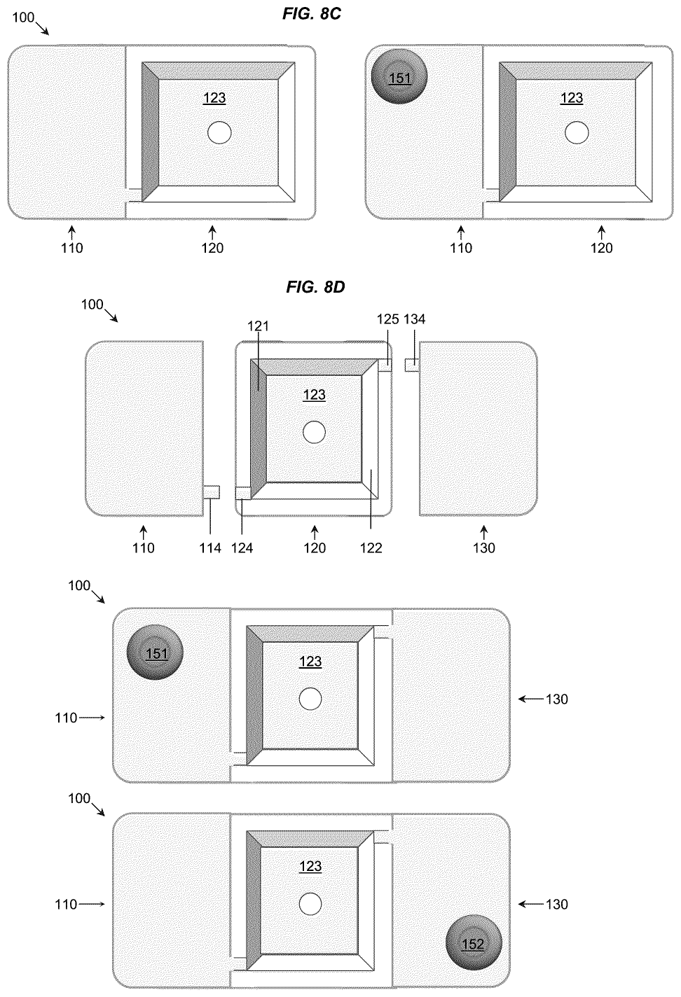

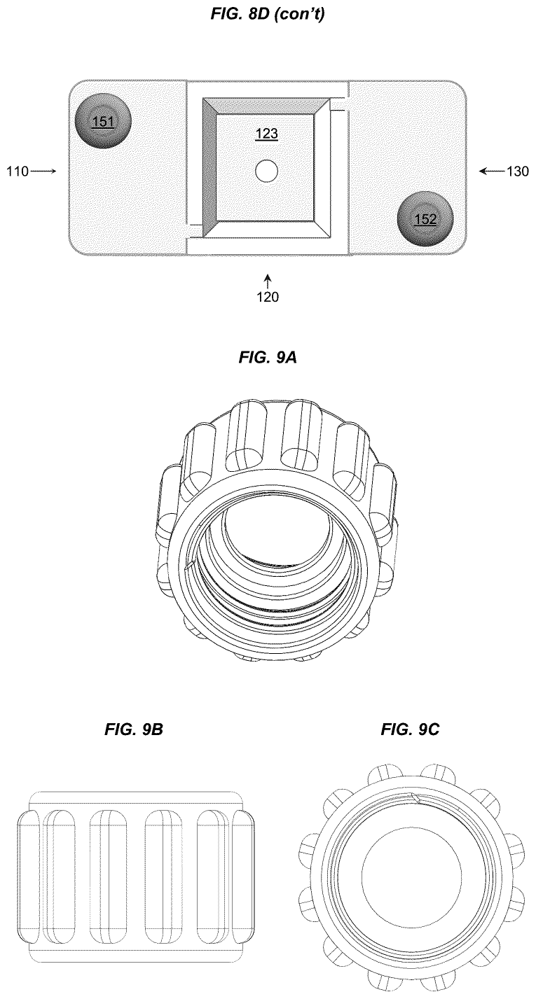

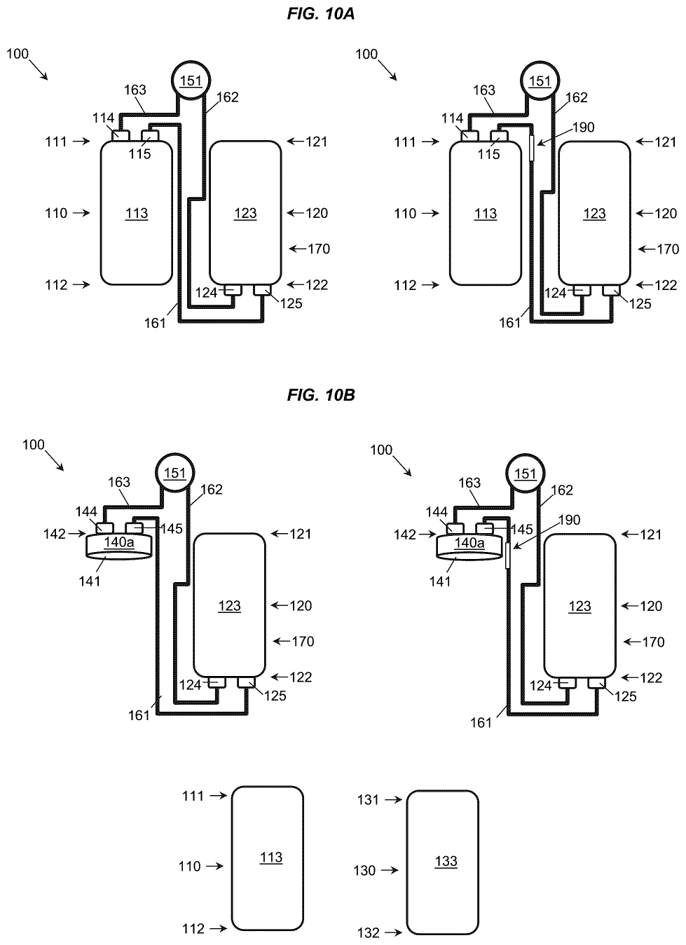

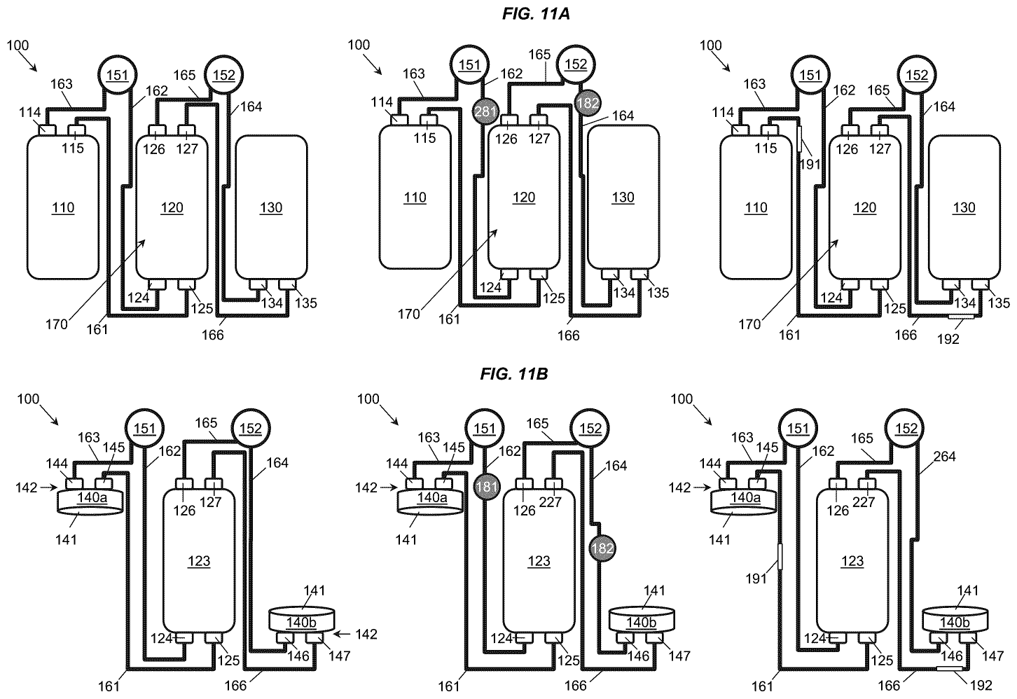

Referring to FIGS. 8A and 8B, the device (1100) may comprise a binding chamber (1120) comprising a binding component (1170), e.g., capable of binding nucleic acid, disposed therein, and either a first coupler (1140a) (opening) or both a first coupler (1140a) and a second coupler (1140b). The couplers (openings) provide access to the binding chamber (1120).

In some embodiments, the first coupler (1140a) and the second coupler (1140b) are configured to engage (e.g., temporarily engage) a closing means, e.g., a component for closing the couplers and preventing access to the binding chamber (1120). In some embodiments, the closing means comprises a cap (1149). In some embodiments, the cap (1149) is removably attached to the coupler. The cap (1149) may move between at least an open position to provide access to the binding chamber (1120) and a closed position where the binding chamber (1120) is not accessible. For example, a cap (1149) may be removably attached to the first coupler (1140a) such that when the cap (1149) is removed from the first coupler (1140a) is in an open position and the binding chamber (1120) is accessible. Additionally, when the cap (1149) is attached to the first coupler (1140a), the first coupler (1140a) is in a closed position, and the first chamber (1210) is not accessible. In some embodiments, when both the first coupler (1140a) and the second coupler (1140b) are engaged with a cap (1149), an enclosed system is created. The present invention is not limited to a cap as a closing means. Other appropriate components may be used, including but not limited to plugs, seals, wax, films, valves, etc.

The device (1100) may further comprise a first vial (1110; e.g., a sample vial) comprising a first end and a second end. The first end of the first vial (1110) is removably attachable to the first coupler (1140a). The first vial (1110) further comprises a lysis buffer. In some embodiments, the first vial (1110) further comprises a lysis buffer and a sample, e.g., a biological sample or an environmental sample. In other embodiments, the device (1100) may comprise a waste vial. The waste vial may be removably attachable to the first coupler (1140a) and is configured to remove a fluid (e.g., the sample) from the binding chamber. In other embodiments, device (1100) may further comprise a second vial (1120; e.g., an elution vial) comprising a first end and a second end. In certain embodiments, the first end of the second vial (1130) is removably attachable to the second coupler (1140b). In other embodiments, the first end (1121) of the second vial (1130) is removably attachable to the first coupler (1140a). The second vial (1130) may comprise an elution buffer.

In accordance with the present invention, the binding chamber (1120) may vary in size and shape depending on the sample size and the type of sample to be processed using the devices described herein. Additionally, the binding component (1170) may vary in size and/or shape to appropriately fit into the binding chamber (1120) and/or optimize binding of nucleic acid (or other molecule, e.g., biomolecule, of interest). Likewise, the binding chamber (1120) may vary in size and/or shape to appropriately fit the binding component (1170) and/or optimize binding of nucleic acid (or other molecule, e.g., biomolecule, of interest).

Referring to FIGS. 8C and 8D, the present invention features a system (100) for isolating nucleic acids from a sample, the system comprising: a) a binding chamber (120), the binding chamber comprising: i) a binding component (170) disposed in an inner cavity (123) of the binding chamber (120), the binding component (170) may be capable of binding nucleic acid; ii) a first binding chamber port (124) disposed in the binding chamber (120) for providing access to the inner cavity (123) of the binding chamber (120); and b) a sample chamber (110) having an inner cavity (113) for housing a fluid sample (e.g., a biological or environmental sample, a test sample or a buffer), the sample chamber (110) comprises a sample chamber port (114) for providing access to the inner cavity (113) of the sample chamber (110), the sample chamber port (114) and the first binding chamber port (124) are adapted to engage so as to fluidly connect the inner cavity (113) of the sample chamber (110) and the inner cavity (123) of the binding chamber (120); and the sample chamber (110) either functions as an actuator or comprises an actuator component (151) to facilitate movement of fluid from the inner cavity (113) of the sample chamber (110) to the inner cavity (123) of the binding chamber (120) or from the inner cavity (123) of the binding chamber (120) of the inner cavity (113) of the sample chamber (110). For example, the sample chamber (110) may be squeezable so as to direct fluid from the sample chamber to the binding chamber.

In some embodiments, the binding chamber (120) may further comprise a second binding chamber port (125) disposed for providing access to the inner cavity (123) of the binding chamber. The first binding chamber port (124) being positioned on an opposite side of the binding chamber (120) to the second binding chamber port (125). For example, in some embodiments, the first binding chamber port (124) is disposed on a first end (121) of the binding chamber (120), and the second binding chamber port (125) is disposed on a second end (122) of the binding chamber (120); wherein the second end (122) is opposite the first end (121).

The system (100) may further comprise a second chamber (130) having an inner cavity (133) for housing a fluid sample (e.g., a wash buffer or an elution buffer). The second chamber (130) comprises a second chamber port (134) for providing access to the inner cavity (133) of the second chamber (130), the second chamber port (134) and the second binding chamber port (125) are adapted to engage so as to fluidly connect the inner cavity (133) of the second chamber (130) and the inner cavity (123) of the binding chamber (120); and the second chamber (130) either functions as an actuator or comprises an actuator component (152) to facilitate movement of fluid from the inner cavity (133) of the second chamber (130) to the inner cavity (123) of the binding chamber (120) or from the inner cavity (123) of the binding chamber (120) of the inner cavity (133) of the second chamber (130). For example, the second chamber (130) may be squeezable so as to direct fluid from the second chamber to the binding chamber.

In some embodiments, at least a portion of the sample chamber (110) is constructed from a flexible material to allow the sample chamber to be squeezed. In some embodiments, the first binding chamber port (124) and/or the sample chamber port (114) is configured to move between at least an open position to allow fluid flow between the sample chamber and the binding chamber (120) and a closed position to prevent fluid flowing between the sample chamber and the binding chamber (120). For example, the first binding chamber port (124) may comprise a valve that occupies a closed position when the first sample chamber port is not engaged with the first binding chamber port. In some embodiments, at least a portion of the second chamber is constructed from a flexible material to allow the chamber to be squeezed. In some embodiments, the second binding chamber port and/or the second chamber port is configured to move between at least an open position to allow fluid flow between the second chamber and the binding chamber (120) and a closed position to prevent fluid flowing between the second chamber and the binding chamber (120). For example, the second binding chamber port may comprise a valve that occupies a closed position when the second chamber port is not engaged with the second binding chamber port.

Referring to FIG. 9A, FIG. 9B, and FIG. 9C, the systems and devices herein may utilize an adapter to facilitate the attachment of a container, tube, or vial (e.g., a sample vial, a collection vial, etc.) or other vessel or component to an attachment point (e.g., coupler, port, channel, hole, valve, etc.) on the system or device, e.g., to connect directly or indirectly the container, tube, vial, or vessel to a fluid chamber. The adapter may be configured to provide a secure and leak-free connection between the container, tube, vial, or vessel and its attachment point on the device or system. In some embodiments, the adapter may comprise one or more filters (e.g., one filter, two filters, three filters, more than three filters, a plurality of filters, etc.) for filtering fluid flowing between the container, tube, vial, or vessel and the fluid chamber. In some embodiments, the adaptor is configured without a filter. In some embodiments, the filters (e.g., two filters, three filters, plurality of filters) are separated, e.g., spaced a distance apart, within the adapter. In some embodiments, the filters are evenly spaced within the adapter, however the present invention is not limited to this configuration. As a non-limiting example, the filters may be separated by one or more spacing means (e.g., spacers, notches, gaskets, o-rings, etc.), or the filters may be immobilized in the adapter in a configuration such that the filters are not in contact with each other, or a combination thereof. In some embodiments, the filters may be arranged within the adapter such that filters with larger pore sizes are disposed distally to the device/attachment point (e.g., coupler port, channel, hole, valve, etc.), and filters with smaller pore sizes are disposed more proximally to the device/attachment point (e.g., coupler port, channel, hole, valve, etc.). The present invention is not limited to this configuration.

Referring to FIG. 10A, the present invention features a system (100) for isolating nucleic acid, e.g., extracting and isolating nucleic acid, from a sample. The system (100) may comprise a first vial (110) having a first end (111) and an inner cavity (113) for housing a sample, a binding chamber (120) comprising an inner cavity (123), a first end (121), a second end (122), and a binding component (170), e.g., capable of binding nucleic acid, disposed in the inner cavity (123) between the first end (121) and the second end (122), a first actuator (151), and a plurality of channels. In some embodiments, the first end (111) of the first vial (110) comprises or is fluidly connected to a first sample vial port (114) and a second sample vial port (115). In some embodiments, the second end (122) of the binding chamber (120) comprises or is fluidly connected to a first binding chamber port (124), and a second binding chamber port (125). A first channel (161) may be fluidly connected to the second sample vial port (115) and the second binding chamber port (125). A second channel (162) may be fluidly connected to the first binding chamber port (124), and a third channel (163) may be fluidly connected to the first sample vial port (114). In some embodiments, the first actuator (151) fluidly connects the second channel (162) to the third channel (163). When activated, the actuator (151) provides pressure for either (i) moving fluid, air, or both from the sample vial (110) to the binding chamber (120) via the first channel (163) and from the binding chamber (120) to the sample vial (110) via the second channel (162) and third channel (163); or (ii) moving fluid, air, or both from the sample vial (110) to the binding chamber (120) via the second channel (162) and third channel (163) and from the binding chamber (120) to the sample vial (110) via the first channel (162). As previously discussed, the methods, systems and devices (e.g., binding component) may be modified to isolate a specific nucleic acid. The methods, systems and devices (e.g., binding component) may be modified to isolate an alternative molecule, e.g., biomolecule.

In some embodiments, fluid moving from the first binding chamber port (124) to the second binding chamber port (125) necessarily passes through or contacts the binding component (170). In some embodiments, the first actuator (151) is a pressure pump.

Referring to FIG. 10B, the present invention also features a system (100) for isolating nucleic acid, e.g., extracting and isolating nucleic acid, from a sample (e.g., a biological sample), wherein the system may comprise a binding chamber (120) comprising a first end (121), a second end (122), and a binding component (170), e.g., capable of binding nucleic acid, disposed therein, a first actuator (151) fluidly connected to the second end (122) of the binding chamber (120), and a coupler fluidly connected to the second end (122) of the binding chamber (120) and fluidly connected to the first actuator (151).

The system (100) may further comprise a first vial (110; e.g., a sample vial) comprising a first end (111) and a second end (112), wherein the first end (111) of the first vial (110) is removably attachable to the outer side (141) of the coupler (140a). The first vial (110) may further comprise a lysis buffer. In some embodiments, the first vial (110) further comprises a lysis buffer and a sample (e.g., environmental sample, biological sample, etc.). Alternatively, the system (100) may comprise a second vial (130; e.g., a collection vial) comprising a first end (131) and a second end (132), wherein the first end (131) of the second vial (130) is removably attachable to the outer side (141) of the coupler (140a). In some embodiments, the second vial (130) may comprise an elution buffer. In other embodiments, the second vial (130) may comprise a wash buffer.

The coupler (140a) may comprise an inner side (142) and an outer side (141). In some embodiments, the coupler (140a) may further comprise a filter (e.g., a prefilter) disposed therein. The filter may allow for filtering cell debris, including but not limited to a cell wall, biological contaminants, pieces of ground tissue, or a combination thereof. The filter may be configured such that the flow of fluid from the first vial (110; e.g., the sample vial) to the binding chamber (220) is through only the filter. In other embodiments, the filter may be configured such that the flow of fluid from the coupler (140a) to the binding chamber (120) is through only the filter. A plurality of filters may be used in accordance with the present invention as long as the filters do not obstruct the flow of fluid from the first vial (110; e.g., the sample vial) or the coupler (140a) to the binding chamber (120).

In some embodiments, the second end (122) of the binding chamber (120) comprises or is fluidly connected to a first binding chamber port (124), and a second binding chamber port (125). In some embodiments, the inner side (142) of the coupler (140, 240) comprises or is fluidly connected to a first coupler port (144) and a second coupler port (145).

In some embodiments, the first actuator (151), the coupler (140a), and the binding chamber (120) are fluidly connected via a plurality of channels. For example, a first channel (161) may fluidly connect the second coupler port (145) to the second binding chamber port (125), a second channel fluidly connects the first binding chamber port (124) to the first actuator (151), and a third channel fluidly connects first actuator (151) to the first coupler port (144). In some embodiments, the first channel (161) may further comprise a filter (190) disposed therein (e.g., an inline filter; see FIGS. 10A and 10B, right side).

In some embodiments, the plurality of channels comprises a plurality of tubes. In other embodiments, the plurality of channels are molded channels.

In some embodiments, the first actuator (151) is fluidly connected to the binding chamber (120) directly. In other embodiments, the first actuator (151) is fluidly connected to the binding chamber (120) indirectly. In some embodiments, the first actuator (151) comprises a one-way valve. In some embodiments, the first actuator (151) is a pressure pump. In some embodiments, the first actuator (151) comprises a directional guide component. The directional guide component may be a valve (e.g., a one-way valve). In some embodiments, the directional guide component is a component that is adapted to allow gravity to guide fluid flow.

In accordance with the present invention, the binding chamber (120) may vary in size and shape depending on the sample size and the type of sample to be processed using the systems described herein. Additionally, the binding component (170) may vary in size to appropriately fit into the binding chamber (120). Likewise, the binding chamber (120) may vary in size to appropriately fit into the binding component (170).

In some embodiments, the binding chamber (120) comprises a cap. The cap may move between at least an open position to provide access to the inner cavity (123) of the binding chamber (120) or the binding component (170), or both, and a closed position where the inner cavity (123) of the binding chamber (120) and binding component (170) are not accessible. In some embodiments, the binding component (170) is attached to the cap.

The present invention also features a method of isolating nucleic acid, e.g., extracting and isolating nucleic acid, from a sample with a system (100) described herein, wherein the method may comprise attaching a first end (111) of a first vial (110; e.g., a sample vial) comprising a lysis buffer and a sample to the outer side (141) of the coupler (140a) and rotating the system (100) into a second position. The second position allows the lysis buffer and the sample to contact the coupler (140; e.g., the first coupler port (144) and the second coupler port (145)). The method may further comprise pressing the first actuator (151) at least once. Pressing the first actuator (151) when the system (100) is in the second position allows the lysis buffer and the sample to move from the first vial (110) into the binding chamber (120), e.g., via the first channel (161). The method may further comprise rotating the system (100) into a first position and pressing the first actuator (151) at least once. Pressing the first actuator (151) when the system (100) is in the first position allows the lysis buffer and the sample to move from the binding chamber (120) into the first vial (110), e.g., via the second channel (162) and the third channel (163). The first vial (110) may be detached from the outer side (141) of the coupler (140a), and a first end (131) second vial (130) comprising a second fluid (e.g., an elution buffer or a wash buffer) may be attached to the outer side (141) of the coupler (140a). The method may comprise rotating the system (100) into the second position and pressing the first actuator (151) at least once. Pressing the first actuator (151) when the system (100) is in the second position allows the second fluid to move from the second vial (130) into the binding chamber (120), e.g., via the first channel (161). The system may then be rotated into the first position and the first actuator (151) at least once. Pressing the first actuator (151) when the system (100) is in the first position allows the second fluid to move from the binding chamber (120) into the second vial (130), e.g., via the second channel (162) and the third channel (163).

In some embodiments, the second fluid may comprise an elution buffer. In other embodiments, the second fluid may comprise a wash buffer.

Referring to FIG. 11A, the present invention also features a system (100) comprising a sample vial (110) having a first end (111) and an inner cavity (113) for housing a sample, a binding chamber (120) comprising an inner cavity (123), a first end (121), a second end (122), and a binding component (170), e.g., capable of binding nucleic acid, disposed in the inner cavity (123) between the first end (121) and the second end (122), a first actuator (151), a second vial (130; e.g., a collection vial) having a first end (131) and an inner cavity (133) for housing a fluid, a second actuator (152), and a plurality of channels. In some embodiments, the first end (111) of the sample vial (110) comprises or is fluidly connected to a first sample vial port (114) and a second sample vial port (115). In some embodiments, the second end (122) of the binding chamber (120) comprises or is fluidly connected to a first binding chamber port (124) and a second binding chamber port (125), and the first end (121) of the binding chamber (120) comprises or is fluidly connected to a third binding chamber port (126) and a fourth binding chamber port (127). A first channel (161) may fluidly connect to the second sample vial port (115) and the second binding chamber port (125). A second channel (162) may fluidly connect to the first binding chamber port (124), and a third channel (163) may fluidly connect to the first sample vial port (115). The first actuator (151) fluidly connects the second channel (162) to the third channel (163). In some embodiments, when activated, the first actuator (151) provides pressure for either (i) moving fluid, air, or both from the sample vial (110) to the binding chamber (120) via the first channel (161) and from the binding chamber (120) to the sample vial (110) via the second channel (162) and third channel (163); or (ii) moving fluid, air, or both from the sample vial (110) to the binding chamber (120) via the second channel (162) and third channel (163) and from the binding chamber (120) to the sample vial (110) via the first channel (161). In some embodiments, the first end (131) of the second vial (130; e.g., a collection vial) comprises or is fluidly connected to a first vial port (134) and a second vial port (135). A fourth channel (164) may fluidly connect to the first vial port (134). A fifth channel (165) may fluidly connect to the third binding chamber port (126), and a sixth channel (166) may fluidly connect to the fourth binding chamber port (127) and the second vial port (135). In some embodiments, the second actuator (152) fluidly connects the fourth channel (164) to the fifth channel (165). In some embodiments, when activated, the second actuator (152) provides pressure for either (i) moving fluid, air, or both from the second vial (230; e.g., a collection vial) to the binding chamber (120) via the fourth channel (164) and fifth channel (165) and from the binding chamber (120) to the second vial (130; e.g., a collection vial) via the sixth channel (166); or (ii) moving fluid, air, or both from the second vial (130; e.g., a collection vial) to the binding chamber (120) via the sixth channel (166) and from the binding chamber (120) to the second vial (130; e.g., a collection vial)) via the fourth channel (164) and fifth channel (165).

Referring to FIG. 11B, the present invention may further feature a system (100) comprising a binding chamber (120) comprising a first end (121), a second end (122), and a binding component (170), e.g., capable of binding nucleic acid, disposed therein, a first actuator (151) fluidly connected to the second end (122) of the binding chamber (120), a first coupler (140a) fluidly connected to the second end (122) of the binding chamber (120) and fluidly connected to the first actuator (151), a second actuator (152) fluidly connected to the first end (121) of the binding chamber (120) and a second coupler (140b) fluidly connected to the first end (121) of the binding chamber (120) and fluidly connected to the second actuator (152).

In some embodiments, fluid moving from the first binding chamber port (124) to the second binding chamber port (125) or from the second binding chamber port (125) to the first binding chamber port (124) necessarily passes through the binding component (1270). In some embodiments, fluid moving from the third binding chamber port (126) to the fourth binding chamber port (127) or from the fourth binding chamber port (127) to the third binding chamber port (126) necessarily passes through the binding component (170).

In some embodiments, the systems (100) described herein may further comprise one or more valves (180). In other embodiments, the systems described herein may comprise a first valve (181) and a second valve (182) (see FIGS. 11A and 11B, middle).

For example, referring to FIG. 11A (middle), the present invention also features a system (100) comprising a sample vial (110) having a first end (111) and an inner cavity (113) for housing a sample, a binding chamber (120) comprising an inner cavity (123), a first end (121), a second end (122), and a binding component (170), e.g., capable of binding nucleic acid, disposed in the inner cavity (123) between the first end (121) and the second end (122), a first actuator (151), a second vial (130; e.g., a collection vial) having a first end (131) and an inner cavity (133) for housing a fluid, a second actuator (152), a first and second valve (181, 182), and a plurality of channels. In some embodiments, the first end (111) of the sample vial (110) comprises or is fluidly connected to a first sample vial port (114) and a second sample vial port (115). In some embodiments, the second end (122) of the binding chamber (120) comprises or is fluidly connected to a first binding chamber port (124) and a second binding chamber port (125), and the first end (121) of the binding chamber (120) comprises or is fluidly connected to a third binding chamber port (126) and a fourth binding chamber port (127). A first channel (161) may fluidly connect to the second sample vial port (115) and the second binding chamber port (125). A second channel (162) may fluidly connect to the first binding chamber port (124), and a third channel (163) may fluidly connect to the first sample vial port (115). The first valve (181) may be disposed on or near the second channel (162) and may inhibit the flow of fluid through the second channel (162). The first actuator (151) fluidly connects the second channel (162) to the third channel (163). In some embodiments, when activated, the first actuator (151) provides pressure for either (i) moving fluid, air, or both from the sample vial (110) to the binding chamber (120) via the first channel (161) and from the binding chamber (120) to the sample vial (110) via the second channel (162) and third channel (163); or (ii) moving fluid, air, or both from the sample vial (110) to the binding chamber (120) via the second channel (162) and third channel (163) and from the binding chamber (120) to the sample vial (110) via the first channel (161). In some embodiments, the first end (131) of the second vial (130; e.g., a collection vial) comprises or is fluidly connected to a first vial port (134) and a second vial port (135). A fourth channel (164) may fluidly connect to the first vial port (134). The second valve (182) may be disposed on or near the fourth channel (164) and inhibit the flow of fluid through the fourth channel (164). A fifth channel (165) may fluidly connect to the third binding chamber port (126), and a sixth channel (166) may fluidly connect to the fourth binding chamber port (127) and the second vial port (135). In some embodiments, the second actuator (152) fluidly connects the fourth channel (164) to the fifth channel (165). In some embodiments, when activated, the second actuator (152) provides pressure for either (i) moving fluid, air, or both from the second vial (130; e.g., a collection vial)) to the binding chamber (120) via the fourth channel (164) and fifth channel (165) and from the binding chamber (120) to the second vial (130; e.g., a collection vial) via the sixth channel (166); or (ii) moving fluid, air, or both from the second vial (130; e.g., a collection vial)) to the binding chamber (120) via the sixth channel (166) and from the binding chamber (120) to the second vial (130; e.g., a collection vial)) via the fourth channel (164) and fifth channel (165).

Referring to FIG. 11B (middle), the present invention also features a system (100) comprising a binding chamber (120) comprising a first end (121), a second end (122), and a binding component (170), e.g., capable of binding nucleic acid, disposed therein, a first actuator (151) fluidly connected to the second end (122) of the binding chamber (120), a first coupler (140a) fluidly connected to the second end (122) of the binding chamber (120) and fluidly connected to the first actuator (151), a first valve (181), a second actuator (152) fluidly connected to the first end (121) of the binding chamber (120), a second coupler (140b) fluidly connected to the first end (121) of the binding chamber (120) and fluidly connected to the second actuator (152), and a second valve (182).

In some embodiments, the first valve (181) disposed between the binding chamber (120) and the first actuator (151) and is capable of inhibiting the fluid connection between the binding chamber (120) and the first actuator (151). In some embodiments, the second valve (182) disposed between the second coupler (140b) and the second actuator (152) and is capable of inhibiting the fluid connection between the second coupler (140b) and the second actuator (152).

In some embodiments, the first end (121) of the binding chamber (120) comprises or is fluidly connected to a third binding chamber port (126) and a fourth binding chamber port (127), and the second end (122) of the binding chamber (120) comprises or is fluidly connected to a first binding chamber port (124) and a second binding chamber port (125).

The first and second couplers may comprise an inner side (142) and an outer side (141). The inner side (142) of the first coupler (140a) comprises or is fluidly connected to a first coupler port (144) and a second coupler port (145), and the inner side (142) of the second coupler (140b) comprises or is fluidly connected to a third coupler port (146) and a fourth coupler port (147). In some embodiments, the first coupler (140a) comprises a filter (e.g., a prefilter) disposed therein. In other embodiments, the first coupler (140a), the second coupler (140b), or both the first and second coupler (140b) comprise a filter disposed therein.

In some embodiments, the first actuator (151), the first coupler (140a), and the binding chamber (120), and the second actuator (152), the second coupler (140b), and the binding chamber (120) are fluidly connected via a plurality of channels. For example, a first channel (161) may fluidly connect the second coupler port (145) to the second binding chamber port (125), a second channel (162) may fluidly connect the first binding chamber port (124) to the first actuator (151), and a third channel (163) fluidly connects the first actuator (151) to the first coupler port (144). Likewise, a fourth channel (164) may fluidly connect the third coupler port (146) to the second actuator (152), a fifth channel (165) may fluidly connect the third binding chamber port (126) to the second actuator (152), and a sixth channel (166) may fluidly connect the fourth binding chamber port (127) to the fourth coupler port (147). In some embodiments, the first channel (161) may comprise a filter (190; e.g., a first filter (191); e.g., an inline filter) disposed therein. In some embodiments, the sixth channel (166) may comprise a filter (190; e.g., a second filter (191); e.g., an inline filter) disposed therein. In other embodiments, the first channel (161) and the sixth channel (166) may comprise a filter (190) disposed therein (e.g., a first filter (191) disposed within the first channel (161) and a second filter (192) disposed within the sixth channel (166)). See FIGS. 8A and 8B (right).

In some embodiments, the system (100) further comprises a first vial (110; e.g., a sample vial) comprising a first end (111) and a second end (112). The first end (111) of the first vial (110) is removably attachable to the outer side (141) of the first coupler (140a). The first vial (110) further comprises a lysis buffer. In some embodiments, the first vial (110) further comprises a lysis buffer and a sample, e.g., a biological sample or an environmental sample. In other embodiments, the system (100) further comprises a second vial (130; e.g., a collection vial) comprising a first end (131) and a second end (132). The first end (131) of the second vial (130) is removably attachable to the outer side (141) of the second coupler (140b). In some embodiments, the second vial (120) comprises an elution buffer. In other embodiments, the second vial (120) comprises a wash buffer.

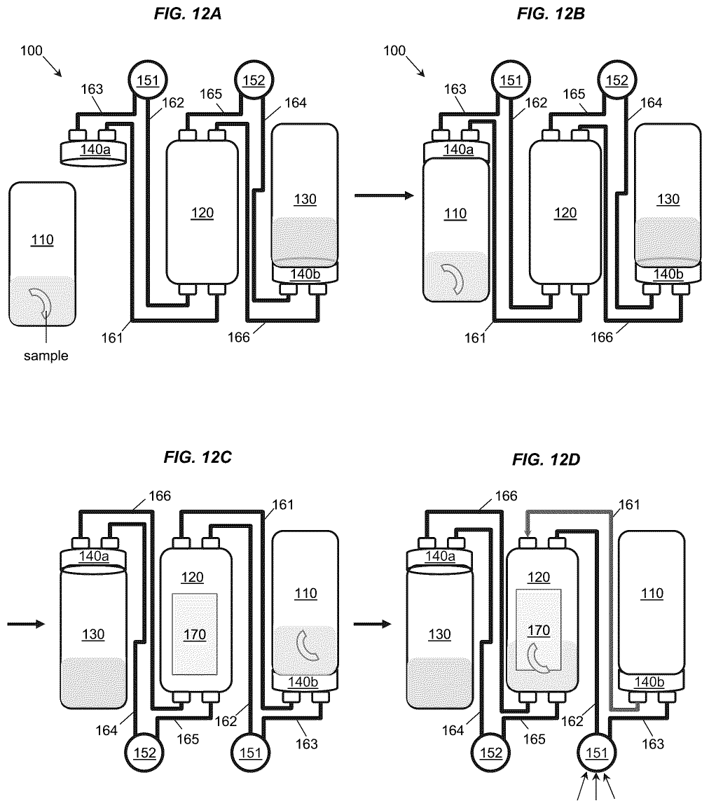

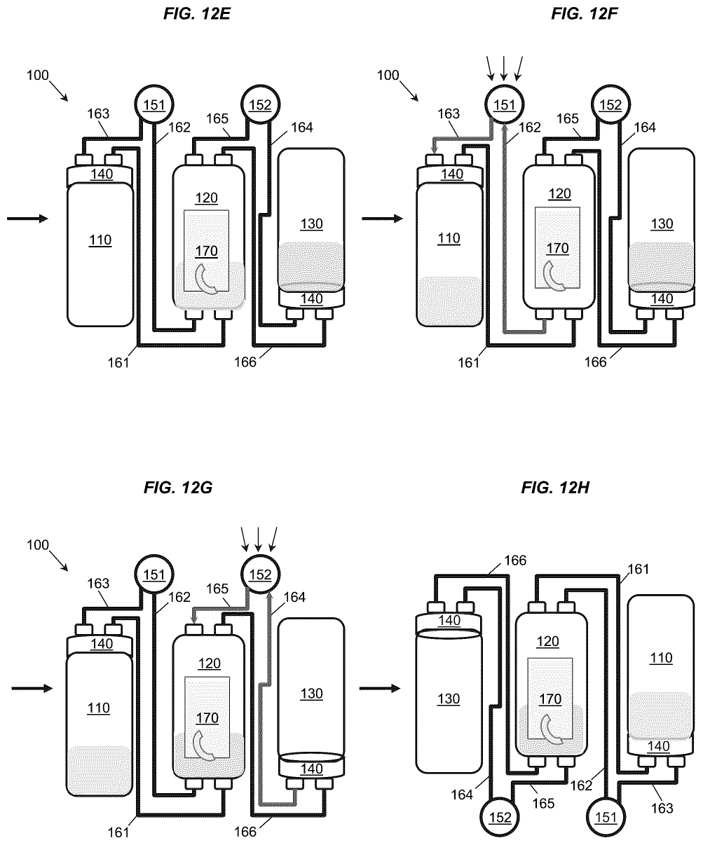

Referring to FIG. 12A-121, the present invention also features a method of isolating nucleic acid, e.g., extracting and isolating nucleic acid, from a sample with a system (100) described herein, wherein the method comprises attaching the first end (111) of the first vial (110) comprising a lysis buffer and a sample to the outer side (141) of the first coupler (140a) and rotating the system (100) into a second position. When the system is in the second position, the lysis buffer and the sample contact the first coupler (140; e.g., the first coupler port (144) and the second coupler port (145)). The method further comprises pressing the first actuator (151) at least once. Pressing the first actuator (151) when the system (100) is in the second position allows the lysis buffer and the sample to move from the first vial (110) into the binding chamber (120), e.g., via the first channel (161). The system (100) is then rotated into a first position, and the first actuator (151) is pressed at least once. When the system is in the first position, the lysis buffer and sample contact the second end (122) of the binding chamber (120), e.g., the first binding chamber port (124) and the second binding chamber port (125). Pressing the first actuator (151) when the system (100) is in the first position allows the lysis buffer and the sample to move from the binding chamber (120) into the first vial (110), e.g., via the second channel (162) and third channel (163). Next, the second actuator (152) is pressed at least one time. When the system (100) is in the first position, the second fluid (e.g., an elution buffer) contacts the second coupler (142), e.g., the third coupler port (146), and the fourth coupler port (147). Pressing the second actuator (152) when the system (100) is in the first position allows the second fluid (e.g., elution buffer) to move from the second vial (130) into the binding chamber (120), e.g., via the fourth channel and the fifth channel. The method comprises rotating the system (100) into the second position and pressing the second actuator (152) at least once. When the system is in the second position, the second fluid (e.g., elution buffer) contacts the first end (121) of the binding chamber (120); e.g., the third binding chamber port (126), and the fourth binding chamber port (127). Pressing the second actuator (152) when the system (100) is in the second position allows the second fluid (e.g., elution buffer) to move from the binding chamber (120) into the second vial (130), e.g., via the sixth channel (166).

The present invention may additionally feature a method of isolating nucleic acid, e.g., extracting and isolating nucleic acids, from a sample with a system (100) described herein. The second vial (130; e.g., a collection vial) houses a second fluid (e.g., an elution buffer or wash buffer) in some embodiments. The method may comprise introducing a sample to the sample vial (110) and rotating the system such that the sample contacts the first sample vial port (124) and second sample vial port (125). The first actuator (151) is activated at least once to allow the sample to move to the binding chamber (120). The system (100) is then rotated such that the sample contacts the first binding chamber port (124) and the second binding chamber port (125). The first actuator (151) is then activated again at least one time to allow the sample to move from the binding chamber (120) to the sample vial (110). The system (100) is then rotated such that the second fluid (e.g., the elution buffer or the wash buffer) in the second vial (130) contacts the first vial port (134) and the second vial port (135). Next, the second actuator (152) is activated at least one time to allow the second fluid to move from the second vial (130) to the binding chamber (120). Then the system (100) is rotated such that the second fluid contacts the third binding chamber port (136) and fourth binding chamber port (137). Lastly, the second actuator (152) may be activated at least once to allow the second solution to move from the binding chamber (120) to the second vial (130).

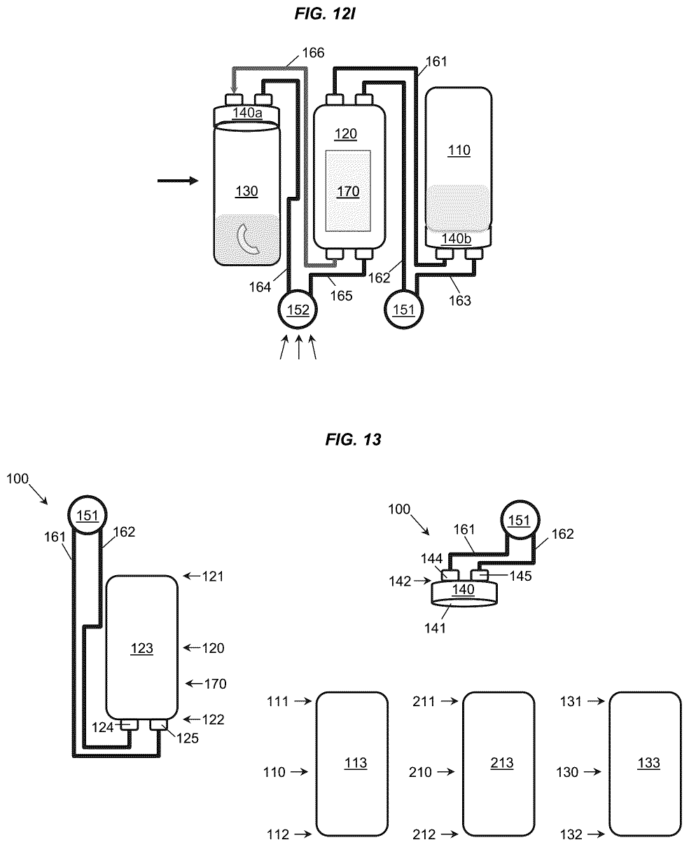

Referring to FIG. 13, in some embodiments, the system (100) may comprise a single bulb and one vial attachment location. In some embodiments, the system comprises a single actuator (151) comprising a one-way valve fluidly connected to a single vial (e.g., a first vial (110); see FIG. 13, left). In other embodiments, the system comprises a single actuator (151) comprising a one-way valve fluidly connected to a coupler (140; see FIG. 13, right). In some embodiments, the system further comprises a vial.

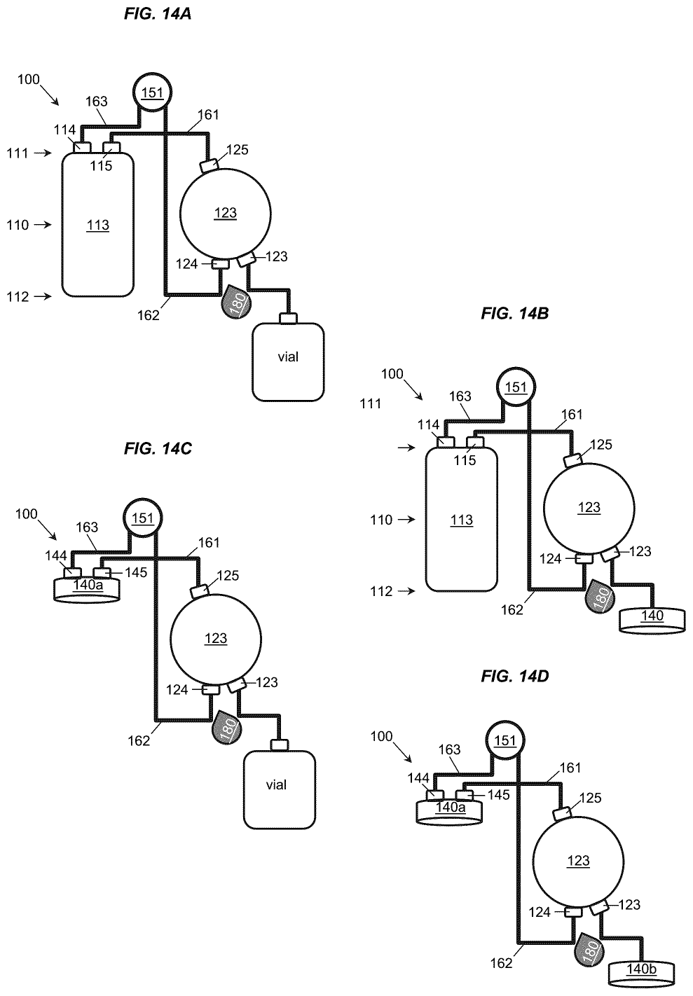

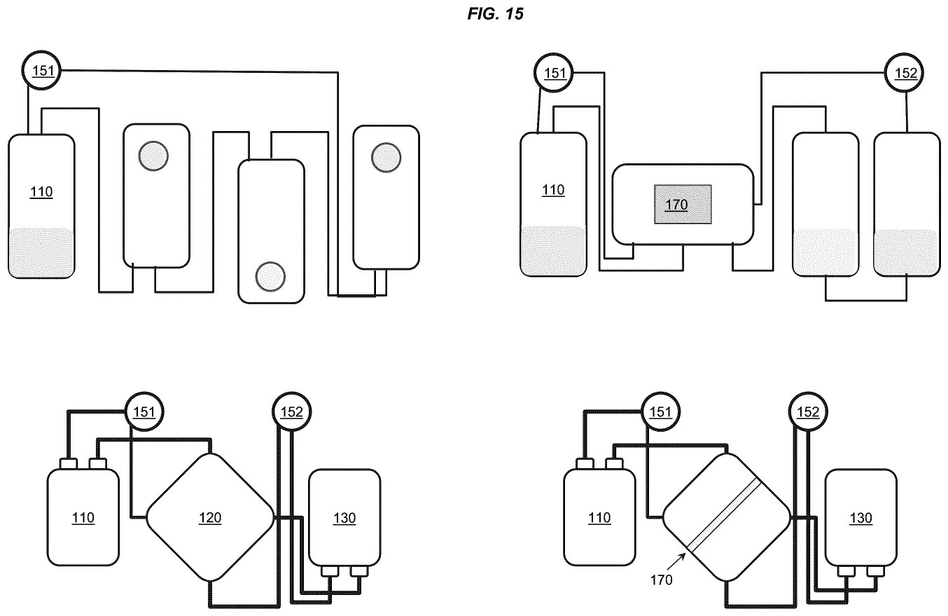

Referring to FIG. 14A-14D, in some embodiments, the present invention features a system for isolating nucleic acids from a sample. In some embodiments, the system (100) comprises a binding chamber (120) comprising a first end (121), a second end (122), and a binding component (170), e.g., capable of binding nucleic acid, disposed therein, a first actuator (151) comprising a one-way valve fluidly connected to the second end (122) of the binding chamber (120), and a first vial (110) fluidly connected to the first end (121) of the binding chamber (120; i.e., the first actuator is fluidly connected to the opposite end of the binding chamber as compared to the first vial (110)) and fluidly connected to the first actuator (151). The system may further comprise a vial (e.g., a waste vial) fluidly connected to the second end (122) of the binding chamber (120). In some embodiments, the binding chamber (120), the first actuator (151), and the first vial (110) are fluidly connected via a plurality of channels, e.g., a first channel (161) may fluidly connect the first vial (110) to the binding chamber (120), a second channel (162) may fluidly connect the first actuator (151) to the binding chamber (120), and a third channel (163) may fluidly connect the first vial (110) to the first actuator (151). In some embodiments, the system further comprises a fourth channel (164) fluidly connecting the binding chamber (120) to the vial (e.g., a waste vial). In further embodiments, the system comprises a valve between the second channel (162) and the fourth channel (164).

In some embodiments, a waste vial may comprise an absorbent material. In some embodiments, the system (100) may further comprise an absorbent material (e.g., an absorbent pad) in lieu of a vial (e.g., a waste vial).

### Supplementary Embodiments of Devices and Systems Herein

In some embodiments, the devices and systems described herein are manufactured from a single molded piece (e.g., a molded piece of plastic). In some embodiments, the molded piece further comprises a film disposed on the mold. The film is configured to seal the mold piece such that it prevents leakage. In some embodiments, the devices and systems described herein are manufactured as modular systems, wherein particular components can be attached together or combined when appropriate.

In some embodiments, the devices and systems described herein are manufactured to allow access to the binding component and/or the binding chamber. For example, the binding chamber may feature an opening that can move or be moved between at least an open position allowing access to the binding chamber, and a closed position preventing access to the binding chamber. In some embodiments, a closing means (e.g., means for preventing access to the binding chamber) moves the opening to the closed position, e.g., temporarily. Non-limiting examples of a closing means are a cap, a valve, a plug, a seal, etc.

In some embodiments, the binding component can be removed from the binding chamber. In some embodiments, the system or device (e.g., the binding chamber) can be opened to access the binding component. In some embodiments, the binding component can be removed via an access door disposed in the device. The access door or slot may be moved between an open position (allowing access to the binding component) and a closed position (preventing access to the binding component). In some embodiments, the binding component is attached to the access door or slot, and the access door or slot can be removed from the device or system. In some embodiments, the access door or slot comprises closing means to prevent access to the binding component, wherein the binding component is attached to the closing means, and the closing means can be removed from the device or system.

In some embodiments, the devices and systems herein feature magnets, e.g., paramagnetic particles. In some embodiments, the devices and systems feature a magnetic chamber, e.g., the magnetic chamber in a lysis circuit.

In some embodiments, the devices and systems feature a heating component operatively connected to or integrated with a portion thereof used for lysis (e.g., cell lysis), optionally before filtration, if applicable. The heating component may help facilitate lysis. In some embodiments, the devices and systems feature a heating component operatively connected to or integrated with a portion thereof used for elution (e.g., elute circuit), which may help with amplification (e.g., isothermal amplification).

In some embodiments, the devices and systems are adapted for the filtration and/or purification of wastewater. In some embodiments, the channel or tube diameters may be altered so as to alter and/or optimize the pressure. In some embodiments, one or more filters are immobile. In some embodiments, one or more filters are mobile.

In some embodiments, devices and systems described herein may be high throughput systems. In other embodiments, the devices and systems described herein may be integrated into a high throughput system, e.g., machines can be used to do the pumping. For example, parts can be attached and detached from machines that do the pumping (e.g., a portion of the housing is configured to be attached and detached from an automated system, e.g., an automated pumping system). In some embodiments, parts can be made smaller to accommodate more samples in a high throughput system. In some embodiments, parts can be made to be disposable.

In some embodiments, the devices and systems described herein may be modular, e.g., various parts may be lined up in a series and attached (such as a stack, for example). In some embodiments, the devices and systems (e.g., stacked forms) may be connected to a pumping machine.

In some embodiments, the nucleic acids isolated using the devices and systems described herein may be used in various downstream applications. For example, the isolated nucleic acids may be used for sequencing.

The devices (1100) or systems (100) described herein are hand-held and/or enclosed.

As used herein, a “port” may refer to a conduit, a hole, a channel, an opening, or the like for the passage of a fluid.

The present invention is not limited to isolation of nucleic acid, e.g., genomic DNA. In some embodiments, the methods, systems, and devices herein may be used to isolate specific nucleic acid by using a binding component comprising material that is capable of binding the specific nucleic acid, e.g., a binding component comprising a probe (e.g., DNA/RNA probe), an aptamer, or other appropriate capture molecule.

The present invention is not limited to isolation of nucleic acid, e.g., genomic DNA, specific nucleic acid of interest, target nucleic acid, etc. The methods, systems, and devices can be modified to isolate other molecules, e.g., biomolecules, or components other than nucleic acid. As a non-limiting example, in some embodiments, the binding component comprises an immobilized antibody specific for a particular antigen in a biological sample. As another non-limiting example, in some embodiments, the binding component comprises additives such as glutathione or another high-affinity molecule so as to configure it to isolate expressed proteins containing tags such as glutathione S-transferase (GST) or other affinity tags.

In some embodiments, the device may be modified and configured so as to be used to perform a biochemical reaction in one of the chambers, wherein the reaction product can be isolated. For example, the binding chamber may contain an immobilized enzyme such as a restriction endonuclease or CRISPR/Cas endonuclease which can cut a specific site in a nucleic acid so that the digested nucleic acid can be isolated.

Such embodiments may also use different kinds of lysis, wash or elution buffers adjusted or optimized based on the biomolecule being isolated.

The device may be used for other methods of known chromatography separation techniques known in art, such as affinity chromatography, paper chromatography.

### Example

The following is a non-limiting example of the present invention. It is to be understood that said example is not intended to limit the present invention in any way. Equivalents or substitutes are within the scope of the present invention.

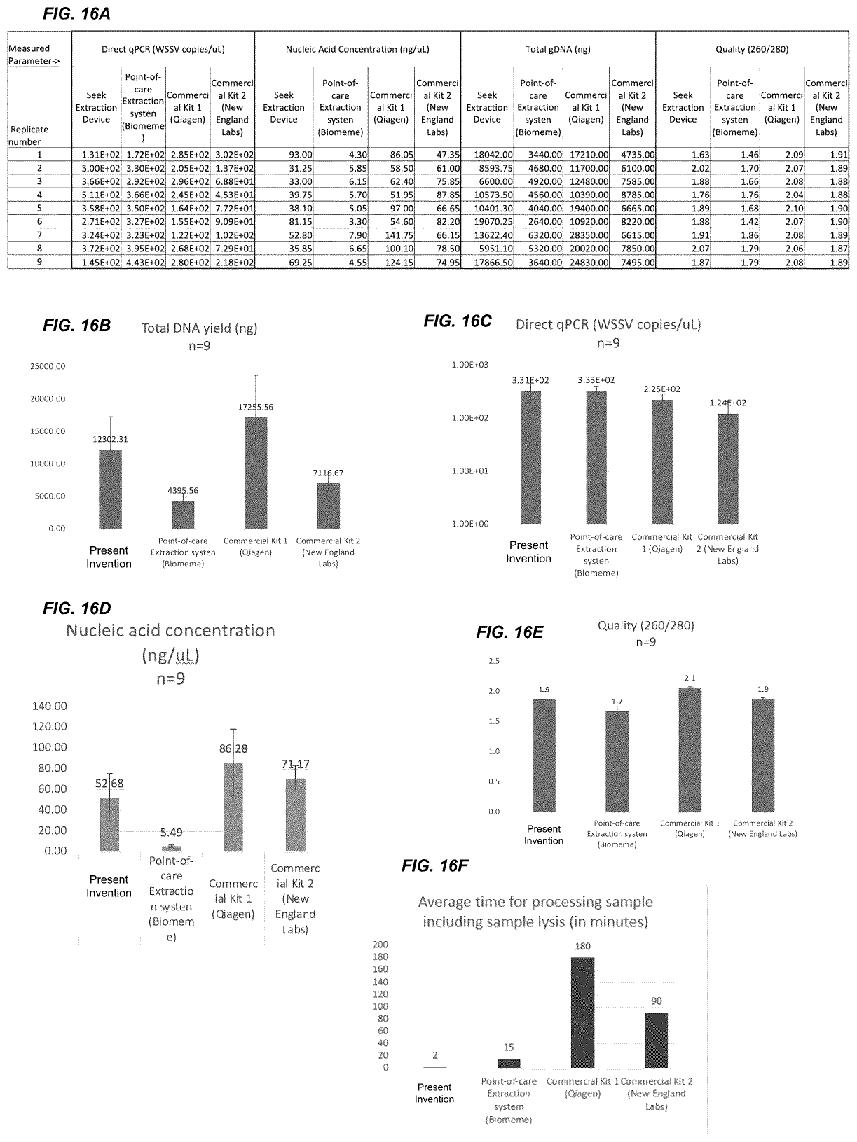

Referring to FIG. 16A-F, multiple shrimps were infected with WSSV virus in a controlled water body. Twenty four hours after infection, the shrimps were isolated and sacrificed. Tissue parts were cut from one of the segments and divided into multiple sections, each weighing 24-25 mg for consistent comparison. Each section was processed by different methods to isolate DNA: a prototype of the present invention described herein, a point-of-use extraction system from Biomeme (M1 Sample prep), a commercial kit from Qiagen, and a commercial kit from New England Biolabs (Monarch). Similar steps were repeated from multiple shrimps from the same set. The DNA from each of these processed samples was measured using UV/vis absorbance spectrophotometer to quantify some parameters of the DNA extracted, such as the concentration in ng/uL and quality as a ratio of 260 nm absorbance to 280 nm absorbance. The total DNA yield in ng was measured by first measuring the amount of elution solution in uL collected in each processing method and multiplying that volume to the DNA concentration from the spectrophotometer. The extracted samples were also used to run qPCR targeting the WSSV VP28 gene using an Agilent AriaMx machine and the WSSV copy numbers were calculated based on standards that were run in the same plate, the standards being known copies of viral DNA. All the values were compared by measuring average and are shown. The standard deviations were measured and used as error bars for the graph comparisons. The devices and systems of the present invention work comparably or better than the other kits. The average processing time for the devices and systems of the present invention is approximately 2 minutes; the average processing time for the alternative point-of-care system was 15 minutes and 90-180 minutes for the commercial DNA isolation kits.

### EMBODIMENTS

The following embodiments are intended to be illustrative only and not to be limiting in any way.

### Embodiment Set A

Embodiment 1A: A system (100) for isolating nucleic acid from a sample, said system (100) comprising: a) a sample vial (110) having a first end (111) and an inner cavity (113) for housing a sample, wherein the first end (111) of the sample vial (110) comprises or is fluidly connected to a first sample vial port (114) and a second sample vial port (115); b) a binding chamber (120) comprising an inner cavity (123), a first end (121), a second end (122), and a binding component (170) capable of binding nucleic acid disposed in the inner cavity (123) between the first end (121) and the second end (122), wherein the second end (122) of the binding chamber (120) comprises or is fluidly connected to a first binding chamber port (124), and a second binding chamber port (125); c) a first channel (161) fluidly connected to the second sample vial port (115) and the second binding chamber port (125); d) a second channel (162) fluidly connected to the first binding chamber port (124); e) a third channel (163) fluidly connected to the first sample vial port (114); and f) a first actuator (151), wherein the first actuator (151) fluidly connects the third channel (163) to the second channel (162), wherein when activated, the first actuator (151) provides pressure for moving fluid, air, or both from the sample vial (110) to the binding chamber (120) via the first channel (161) or from the binding chamber (120) to the sample vial (110) via the second channel (162) and third channel (163).

Embodiment 2A: The system of embodiment 1A, wherein the first actuator (151) is a pressure pump. Embodiment 3A: The system of embodiment 1A, wherein the first actuator (151) comprises a directional guide component. Embodiment 4A: The system of embodiment 3A, wherein the directional guide component is a valve. Embodiment 5A: The system of embodiment 4A, wherein the valve is a one-way valve. Embodiment 6A: The system of embodiment 4A, wherein the directional guide component is a component that is adapted to allow gravity to guide fluid flow. Embodiment 7A: The system of any one of embodiments 1A-6A, wherein the binding component (170) comprises cellulose. Embodiment 8A: The system of any one of embodiments 1A-7A, wherein fluid moving from the first binding chamber port (124) to the second binding chamber port (125) necessarily passes through the binding component (170). Embodiment 9A: The system of any one of embodiments 1A-8A, wherein the sample is a liquid sample. Embodiment 10A: The system of any one of embodiments 1A-9A, wherein the sample comprises a combination of liquid and solid material. Embodiment 11A: The system of any one of embodiments 1A-10A, wherein the sample is a biological sample. Embodiment 12A: The system of any one of embodiments 1A-10A, wherein the sample is an environmental sample.

Embodiment 13A: A system (100) for isolating nucleic acids from a sample, the system (100) comprising: a) a binding chamber (120) comprising a first end (121), a second end (122), and a binding component (170) capable of binding nucleic acid disposed therein; b) a first actuator (151) fluidly connected to the second end (122) of the binding chamber (120); and c) a coupler (comprising an inner side (142) and an outer side (141), wherein the coupler is fluidly connected to the second end (122) of the binding chamber (120) and fluidly connected to the first actuator (151).

Embodiment 14A: The system (100) of embodiment 13A, the second end (122) of the binding chamber (120) comprises or is fluidly connected to a first binding chamber port (124), and a second binding chamber port (125). Embodiment 15A: The system (100) of embodiment 13A or embodiment 14A, wherein the inner side (142) of the coupler comprises or is fluidly connected to a first coupler port (144) and a second coupler port (145). Embodiment 16A: The system (100) of any one of embodiments 13A-15A, wherein the first actuator (151), binding chamber (120), and the coupler are fluidly connected via a plurality of channels. Embodiment 17A: The system of any one of embodiments 13A-16A, wherein a first channel (161) fluidly connects the second coupler port (145) to the second binding chamber port (125), a second channel (162) fluidly connects the first binding chamber port (124) to the first actuator (151), and a third channel (163) fluidly connects first actuator (151) to the first coupler port (144). Embodiment 18A: The system of any one of embodiments 13A-17A, wherein fluid moving from the first binding chamber port (124) to the second binding chamber port (125) necessarily passes through the binding component (170). Embodiment 19A: The system (100) of any one of embodiments 13A-18A, wherein the binding component (170) comprises cellulose. Embodiment 20A: The system (100) of any one of embodiments 13A-19A, further comprising a first vial (110) comprising a first end (111) and a second end (112), wherein the first end (111) of the first vial (110) is removably attachable to the outer side (141) of the coupler. Embodiment 21A: The system (100) of embodiment 20A, wherein the first vial (110) further comprises a lysis buffer. Embodiment 22A: The system (100) of embodiment 20A or embodiment 21A, wherein the first vial (110) further comprises a sample. Embodiment 23A: The system (100) of any one of embodiments 13A-22A further comprising a second vial (130) comprising a first end (131) and a second end (132), wherein the first end (131) of the second vial (130) is removably attachable to the outer side (141) of the coupler. Embodiment 24A: The system (100) of embodiment 23A, wherein the second vial (130) further comprises an elution buffer. Embodiment 25A: The system of embodiment 13A, wherein the first actuator (151) comprises a directional guide component. Embodiment 26A: The system of embodiment 25A, wherein the directional guide component is a valve. Embodiment 27A: The system of embodiment 26A, wherein the valve is a one-way valve. Embodiment 28A: The system of embodiment 25A, wherein the directional guide component is a component that is adapted to allow gravity to guide fluid flow. Embodiment 29A: The system of any one of embodiments 13A-28A, wherein the sample is a liquid sample. Embodiment 30A: The system of any one of embodiments 13A-29A, wherein the sample comprises a combination of liquid and solid material. Embodiment 31A: The system of any one of embodiments 13A-30A, wherein the sample is a biological sample. Embodiment 32A: The system of any one of embodiments 13A-30A, wherein the sample is an environmental sample.

Embodiment 33A: A method of isolating nucleic acids from a sample with a system (100) according to any one of embodiments 13A-32A, the method comprising: a) attaching the first end (111) of the first vial (110) comprising lysis buffer and a sample to the outer side (141) of the coupler; b) rotating the system (100) into a second position, wherein the second position allows the lysis buffer and the sample to contact the coupler; c) pressing the first actuator (151) at least one time; wherein pressing the first actuator (151) when the system (100) is in the second position allows the lysis buffer and the sample to move from the first vial (110) into the binding chamber (120); and d) rotating the system (100) into a first position and pressing the first actuator (151) at least once, wherein pressing the first actuator (151) when the system (100) is in the first position allows the lysis buffer and the sample to move from the binding chamber (120) into the first vial (110). Embodiment 34A: The method of embodiment 33A further comprising: a) detaching the first vial (100) from the outer side (141) of the coupler and attaching a second vial (130) comprising a second fluid to the outer side (141) of the coupler; b) rotating the system (100) into the second position and pressing the first actuator (151) at least one time; wherein pressing the first actuator (151) when the system (100) is in the second position allows the second fluid to move from the second vial (130) into the binding chamber (120); c) rotating the system (100) into the first position; and d) pressing the first actuator (151) at least one time; wherein pressing the first actuator (151) when the system (100) is in the second position allows the second fluid to move from the binding chamber (120) into the second vial (130).

Embodiment 35A: A system (100) for isolating nucleic acid from a sample, said system (100) comprising: a) a sample vial (110) having a first end (111) and an inner cavity (113) for housing a sample, wherein the first end (111) of the sample vial (110) comprises or is fluidly connected to a first sample vial port (114) and a second sample vial port (115); b) a binding chamber (120) comprising an inner cavity (123), a first end (121), a second end (122), and a binding component (170) capable of binding nucleic acid disposed in the inner cavity (123) between the first end (121) and the second end (122), wherein the second end (122) of the binding chamber (120) comprises or is fluidly connected to a first binding chamber port (124) and a second binding chamber port (125), and the first end (121) of the binding chamber (120) comprises or is fluidly connected to a third binding chamber port (126) and a fourth binding chamber port (127); c) a first channel (161) fluidly connected to the second sample vial port (115) and the second binding chamber port (125); d) a second channel (162) fluidly connected to the first binding chamber port (124); e) a third channel (163) fluidly connected to the first sample vial port (115); f) a first actuator (151), wherein the first actuator (151) fluidly connects the third channel (163) to the second channel (162), wherein when activated, the first actuator (151) provides pressure for moving fluid, air, or both from the sample vial (110) to the binding chamber (120) via the first channel (161) and from the binding chamber (120) to the sample vial (110) via the second channel (162) and third channel (163); g) a second vial (130) having a second end (132) and an inner cavity (133) for housing a fluid, wherein the second end (132) of the second vial (130) comprises or is fluidly connected to a first vial port (134) and a second vial port (135); h) a fourth channel (164) fluidly connected to the first vial port (134); i) a fifth channel (165) fluidly connected to the third binding chamber port (126); j) a sixth channel (166) fluidly connected to the fourth binding chamber port (127) and the second vial port (135); and k) a second actuator (152), wherein the second actuator (152) fluidly connects the fourth channel (164) to the fifth channel (165), wherein when activated, the second actuator (152) provides pressure for moving fluid, air, or both from the from the second vial (130) to the binding chamber (120) via the fourth channel (164) and fifth channel (165) and from the binding chamber (120) to the vial (130) via the sixth channel (166).

Embodiment 36A: The system (100) of embodiment 35A, wherein the first actuator (151), the second actuator (152), or both are a pressure pump. Embodiment 37A: The system (100) of embodiment 35A, wherein the first actuator (151) comprises a directional guide component. Embodiment 38A: The system (100) of embodiment 35A, wherein the second actuator (152) comprises a directional guide component. Embodiment 39A: The system (100) of embodiment 37A or 38A, wherein the directional guide component is a valve. Embodiment 40A: The system (100) of embodiment 39A, wherein the valve is a one-way valve. Embodiment 41A: The system (100) of embodiment 37A or embodiment 38A, wherein the directional guide component is a component that is adapted to allow gravity to guide fluid flow. Embodiment 42A: The system (100) of any one of embodiments 35A-41A, wherein the binding component (170) comprises cellulose. Embodiment 43A: The system (100) of any one of embodiments 35A-42A, wherein fluid moving from the first binding chamber port (124) to the second binding chamber port (125) or from the second binding chamber port (125) to the first binding chamber port (124) necessarily passes through the binding component (170). Embodiment 44A: The system (100) of any one of embodiments 35A-41A, wherein fluid moving from the third binding chamber port (126) to the fourth binding chamber port (127) or from the fourth binding chamber port (127) to the third binding chamber port (126) necessarily passes through the binding component (170). Embodiment 45A: A method of isolating nucleic acids from a sample with a system (100) according to any one of embodiments 35A-44A, wherein the second vial (230) housing elution buffer, the method comprising: a) introducing a sample to the sample vial (110); b) rotating the system (100) such that the sample contacts the first sample vial port (124) and second sample vial port (125); c) activating the first actuator (151) at least one time to allow the sample to move to the binding chamber (120); d) rotating the system (100) such that the sample contacts the first binding chamber port (124) and the second binding chamber port (125); e) activating the first actuator (151) at least one time to allow the sample to move from the binding chamber (120) to the sample vial (110); f) rotating the system (100) such that the elution buffer in the second vial (130) contacts the first vial port (134) and the second vial port (135); g) activating the second actuator (151) at least one time to allow the elution buffer to move from the second vial (130) to the binding chamber (120); h) rotating the system (100) such that the elution buffer contacts the third binding chamber port (136) and fourth binding chamber port (137); i) activating the second actuator (152) at least once to allow the elution buffer to move from the binding chamber (120) to the second vial (130).

Embodiment 46A: A system (100) for isolating nucleic acids from a sample, the system (100) comprising: a) a binding chamber (120) comprising a first end (121), a second end (122), and a binding component (170) capable of binding nucleic acid disposed therein; b) a first actuator (151) fluidly connected to the second end (122) of the binding chamber (120); c) a first coupler (140a) comprising an inner side (142) and an outer side (141), wherein the first coupler (140a) is fluidly connected to the second end (121) of the binding chamber (120) and fluidly connected to the first actuator (151); d) a second actuator (152) fluidly connected to the first end (121) of the binding chamber (120); and e) a second coupler (140b) comprising an inner side (142) and an outer side (141), wherein the second coupler (142) is fluidly connected to the first end (121) of the binding chamber (120) and fluidly connected to the second actuator (152).

Embodiment 47A: The system (100) of embodiment 46A, wherein the second end (122) of the binding chamber (120) comprises or is fluidly connected to a third binding chamber port (126) and a fourth binding chamber port (127), and the first end (121) of the binding chamber (120) comprises or is fluidly connected to a first binding chamber port (124) and a second binding chamber port (125). Embodiment 48A: The system (100) of embodiment 46A or embodiment 47A, wherein the inner side (142) of the first coupler (140a) comprises or is fluidly connected to a first coupler port (144) and a second coupler port (145). Embodiment 49A: The system (100) of any one of embodiments 46A-48A, wherein the inner side (142) of the second coupler (140b) comprises or is fluidly connected to a third coupler port (146) and a fourth coupler port (147). Embodiment 50A: The system (100) of any one of embodiments 46A-49A, wherein the first actuator (151), the first coupler (10) and the binding chamber (120), and the second actuator (152), the second coupler (140b) and the binding chamber (120) are fluidly connected via a plurality of channels. Embodiment 51A: The system (100) of any one of embodiments 46A-50A, wherein a first channel (161) fluidly connects the second coupler port (145) to the second binding chamber port (125), a second channel (162) fluidly connects the first binding chamber port (124) to the first actuator (151), and a third channel (163) fluidly connects the first actuator (151) to the first coupler port (144). Embodiment 52A: The system (100) of any one of embodiments 46A-50A, wherein a fourth channel (164) fluidly connects the third coupler port to the second actuator (152), a fifth channel (165) fluidly connects the third binding chamber port (126) to the second actuator (152), and a sixth channel (166) fluidly connects the fourth binding chamber port (127) to the fourth coupler port (147). Embodiment 53A: The system (100) of any one of embodiments 46A-52A, further comprising a first vial (110) comprising a first end (111) and a second end (112), wherein the first end (111) of the first vial (110) is removably attachable to the outer side (141) of the first coupler (140a). Embodiment 54A: The system (100) of embodiment 53A, wherein the first vial (110) further comprises a lysis buffer. Embodiment 55A: The system (100) of embodiment 53A or embodiment 54A, wherein the first vial (110) further comprises a biological sample or an environmental sample. Embodiment 56A: The system (100) of any one of embodiments 46A-55A further comprising a second vial (130) comprising a first end (131) and a second end (132), wherein the first end (131) of the second vial (130) is removably attachable to the outer side (141) of the second coupler (140b). Embodiment 57A: The system (100) of embodiment 56A, wherein the second vial (130) further comprises an elution buffer. Embodiment 58A: The system (100) of any one of embodiments 46A-57A, wherein the binding component (170) comprises cellulose. Embodiment 59A: The system (100) of any one of embodiments 46A-58A, further comprising one of more valves. Embodiment 60A: The system (100) of any one of embodiments 46A-58A, further comprising a first valve (181) and a second valve (182). Embodiment 61A: The system (100) of any one of embodiments 46A-60A, wherein the first actuator (151) comprises a directional guide component. Embodiment 62A: The system (100) of any one of embodiments 46A-60A, wherein the second actuator (152) comprises a directional guide component. Embodiment 63A: The system (100) of embodiment 60A or 61A, wherein the directional guide component is a valve. Embodiment 64A: The system (100) of embodiment 62A, wherein the valve is a one-way valve. Embodiment 65A: The system (100) of embodiment 60A or 61A, wherein the directional guide component is a component that is adapted to allow gravity to guide fluid flow. Embodiment 66A: The system (100) of any one of embodiments 46A-66A, wherein the sample is a biological sample. Embodiment 67A: The system (100) of any one of embodiments 46A-66A, wherein the sample is an environmental sample.

Embodiment 68A: A method of isolating nucleic acids from a sample with a system (100) according to any one of embodiments 46A-67A, the method comprising: a) attaching the first end (111) of the first vial (110) comprising a lysis buffer and a sample to the outer side (141) of the first coupler (140a); b) rotating the system (100) into a second position, wherein the second position allows the lysis buffer and the sample to contact the first coupler (140a); c) pressing the first actuator (151) at least one time; wherein pressing the first actuator (151) when the system (100) is in the second position allows the lysis buffer and the sample to move from the first vial (110) into the binding chamber (120); d) rotating the system (100) into a first position and pressing the first actuator (151) at least once, wherein pressing the first actuator (151) when the system (100) is in the first position allows the lysis buffer and the sample to move from the binding chamber (120) into the first vial (110); e) pressing the second actuator (152) at least one time; wherein pressing the second actuator (152) when the system (100) is in the first position allows the elution buffer to move from the second vial (130) into the binding chamber (120); f) rotating the system (100) into the second position; and g) pressing the second actuator (152) at least one time; wherein pressing the second actuator (152) when the system (100) is in the second position allows the elution buffer to move from the binding chamber (120) into the second vial (130).

Embodiment 69A: A system (100) for isolating nucleic acids from a sample, the system (100) comprising: a) a binding chamber (120) comprising a first end (121), a second end (122), and a binding component (170) capable of binding nucleic acid disposed therein; b) a first actuator (151) fluidly connected to the second end (122) of the binding chamber (120); c) a first coupler (140a) comprising an inner side (142) and an outer side (141), wherein the first coupler (140a) is fluidly connected to the second end (121) of the binding chamber (120) and fluidly connected to the first actuator (151); d) a first valve (181) disposed between the binding chamber (120) and the first actuator (151), wherein the first valve (181) is capable of inhibiting the fluid connection between the binding chamber (120) and the first actuator (151); e) a second actuator (152) fluidly connected to the first end (121) of the binding chamber (120); f) a second coupler (140b) comprising an inner side (142) and an outer side (141), wherein the second coupler (142) is fluidly connected to the first end (121) of the binding chamber (120) and fluidly connected to the second actuator (152); and g) a second valve (182) disposed between the second coupler (140b) and the second actuator (152), wherein the second valve (182) is capable of inhibiting the fluid connection between the second coupler (140b) and the second actuator (152).

Embodiment 70A: The system (100) of embodiment 69A, wherein the first end (121) of the binding chamber (120) comprises or is fluidly connected to a third binding chamber port (126) and a fourth binding chamber port (127), and the second end (122) of the binding chamber (120) comprises or is fluidly connected to a first binding chamber port (124) and a second binding chamber port (125). Embodiment 71A: The system (100) of embodiment 69A or embodiment 70A, wherein the inner side (142) of the first coupler (140a) comprises or is fluidly connected to a first coupler port (144) and a second coupler port (145). Embodiment 72A: The system (100) of any one of embodiments 69A-71A, wherein the inner side (142) of the second coupler (140b) comprises or is fluidly connected to a third coupler port (146) and a fourth coupler port (147). Embodiment 73A: The system (100) of any one of embodiments 69A-72A, wherein the first actuator (151), the first coupler (140a) and the binding chamber (120), and the second actuator (152), the second coupler (140b) and the binding chamber (120) are fluidly connected via a plurality of channels. Embodiment 74A: The system (100) of any one of embodiments 69A-73A, wherein a first channel (161) fluidly connects the second coupler port (145) to the second binding chamber port (125), a second channel (162) fluidly connects the first binding chamber port (124) to the first actuator (151), a third channel (163) fluidly connects the first coupler port (144) to the first actuator (151). Embodiment 75A: The system (100) of any one of embodiments 69A-73A, wherein a fourth channel (164) fluidly connects the third coupler port to the second actuator (152), a fifth channel (165) fluidly connects the third binding chamber port (126) to the second actuator (152), and a sixth channel (166) fluidly connects the fourth binding chamber port (127) to the fourth coupler port (147). Embodiment 76A: The system (100) of any one of embodiments 69A-75A, wherein the binding component (170) comprises cellulose. Embodiment 77A: The system (100) of any one of embodiments 69A-76A, further comprising a first vial (110) comprising a first end (111) and a second end (112), wherein the first end (111) of the first vial (110) is removably attachable to the outer side (141) of the first coupler (140a). Embodiment 78A: The system (100) of embodiment 77A, wherein the first vial (110) further comprises a lysis buffer. Embodiment 79A: The system (100) of embodiment 77A or embodiment 78A, wherein the first vial (110) further comprises a biological sample or an environmental sample. Embodiment 80A: The system (100) of any one of embodiments 69A-79A further comprising a second vial (130) comprising a first end (131) and a second end (132), wherein the first end (131) of the second vial (130) is removably attachable to the outer side (141) of the second coupler (140b). Embodiment 81A: The system (100) of embodiment 80A, wherein the second vial (130) further comprises an elution buffer. Embodiment 82A: The system (100) of embodiment 80A, wherein the second vial (130) further comprises a wash buffer. Embodiment 83A: The system (100) of any one of embodiments 69A-82A, wherein the first actuator (151) comprises a directional guide component. Embodiment 84A: The system (100) of any one of embodiments 69A-82A, wherein the second actuator (152) comprises a directional guide component. Embodiment 85A: The system (100) of embodiment 83A or embodiment 84A, wherein the directional guide component is a valve. Embodiment 86A: The system (100) of embodiment 85A, wherein the valve is a one-way valve. Embodiment 87A: The system (100) of embodiment 83A or embodiment 84A, wherein the directional guide component is a component that is adapted to allow gravity to guide fluid flow. Embodiment 88A: The system (100) of any one of embodiments 69A-87A, wherein the sample is a biological sample. Embodiment 89A: The system (100) of any one of embodiments 69A-87A, wherein the sample is an environmental sample.

Embodiment 90A: A system (100) for isolating nucleic acid from a sample, said system (100) comprising: a) a sample vial (110) having a first end (111) and an inner cavity (113) for housing a sample, wherein the first end (111) of the sample vial (110) comprises or is fluidly connected to a first sample vial port (114) and a second sample vial port (115); b) a binding chamber (120) comprising an inner cavity (123), a first end (121), a second end (122), and a binding component (170) capable of binding nucleic acid disposed in the inner cavity (123) between the first end (121) and the second end (122), wherein the second end (122) of the binding chamber (120) comprises or is fluidly connected to a first binding chamber port (124) and a second binding chamber port (125), and the second end (122) of the binding chamber (120) comprises or is fluidly connected to a third binding chamber port (126) and a fourth binding chamber port (127); c) a first channel (161) fluidly connected to the second sample vial port (115) and the second binding chamber port (125); d) a second channel (162) fluidly connected to the first binding chamber port (124); e) a first valve (181) disposed on or near the second channel (162), wherein the first valve inhibits the flow of a fluid through the second channel (162); f) a third channel (163) fluidly connected to and the first sample vial port (114); g) a first actuator (151), wherein the first actuator (151) fluidly connects the third channel (163) to the second channel (162), wherein when activated, the first actuator (151) provides pressure for moving fluid, air, or both from the sample vial (110) to the binding chamber (120) via the first channel (161) and from the binding chamber (120) to the sample vial (110) via the second channel (162) and third channel (163); h) a second vial (130) having a second end (132) and an inner cavity (133) for housing a fluid, wherein the second end (132) of the second vial (130) comprises or is fluidly connected to a first vial port (134) and a second vial port (135); i) a fourth channel (164) fluidly connected to the first elution vial port (134); j) a second valve (182) disposed on or near the fourth channel (164), wherein the second valve (182) inhibits the flow of a fluid through the fourth channel (164); k) a fifth channel (165) fluidly connected to the third binding chamber port (126); I) a sixth channel (166) fluidly connected to the fourth binding chamber port (127) and the second elution vial port (135); and m) a second actuator (152), wherein the second actuator (152) fluidly connects the fourth channel (164) to the fifth channel (165), wherein when activated, the second actuator (152) provides pressure for moving fluid, air, or both from the second vial (130) to the binding chamber (120) via the fourth channel (164) and fifth channel (165) and from the binding chamber (120) to the second vial (130) via the sixth channel (166).

Embodiment 91A: The system (100) of embodiment 90A, wherein the binding component (170) comprises cellulose. Embodiment 92A: The system (100) of embodiment 90A or embodiment 91A, wherein the first actuator (151), the second actuator (152), or both are a pressure pump. Embodiment 93A: The system (100) of any one of embodiments 90A-92A, wherein fluid moving from the first binding chamber port (124) to the second binding chamber port (125) necessarily passes through the binding component (170). Embodiment 94A: The system (100) of any one of embodiments 90A-93A, wherein the sample is a liquid sample. Embodiment 95A: The system (100) of any one of embodiments 90A-94A, wherein the sample comprises a combination of liquid and solid material. Embodiment 96A: The system (100) of any one of embodiments 90A-95A, wherein the sample is a biological sample. Embodiment 97A: The system (100) of any one of embodiments 90A-95A, wherein the sample is an environmental sample.

Embodiment 98A: The system (100) of any of the preceding embodiments, wherein the first actuator (151) comprises a directional guide component. Embodiment 99A: The system (100) of any of the preceding embodiments, wherein the second actuator (152) comprises a directional guide component. Embodiment 100A: The system (100) of any of the preceding embodiments, wherein the directional guide component is a valve. Embodiment 101A: The system (100) of any of the preceding embodiments, wherein the valve is a one-way valve. Embodiment 102A: The system (100) of any of the preceding embodiments, wherein the directional guide component is a component that is adapted to allow gravity to guide fluid flow.

Embodiment 103A: A device (1100) for isolating nucleic acid from a sample, said device comprising: a) a binding chamber (1120) having an inner cavity therein (1123), wherein a binding component (1170) capable of binding nucleic acid is disposed in the inner cavity (1123); b) a first channel (1161) having a first end fluidly connected to the binding chamber (1120) and a second end configured to fluidly couple to a first vial (1110); c) a second channel (1162) having a first end fluidly connected to the binding chamber (1120); d) a first actuator (1151) operatively connected to the second channel (1162), the first actuator (1151) is configured to assist movement of a fluid through at least the second channel (1162), the first actuator (1151) is optionally fluidly connected to a second end of the second channel (1162); e) a fifth channel (1165) having a first end fluidly connected to the binding chamber (1120); f) a sixth channel (1166) having a first end fluidly connected to the binding chamber (1120) and a second end configured to fluidly couple to a second vial (1130); g) a second actuator (1152) operatively connected to the fifth channel (1165), the second actuator (1152) is configured to assist movement of a fluid through at least the fifth channel (1165), the second actuator (1152) is optionally fluidly connected to a second end of the fifth channel (1165). Embodiment 104A: The device (1100) of embodiment 103A, wherein the first actuator (1151) is fluidly connected to a third channel (1163). Embodiment 105A: The device (1100) of embodiment 103A or embodiment 104A, wherein the third channel comprises an end configured to fluidly connect to the first vial (1110). Embodiment 106A: The device (1100) of any one of embodiments 103A-105A, wherein the second actuator (1151) is fluidly connected to a fourth channel (1164). Embodiment 107A: The device (1100) of embodiment 106A, wherein the fourth channel (1164) comprises an end configured to fluidly connect to the second vial (1130).

Embodiment 108A: A device (1100) for isolating nucleic acid from a sample, said device comprising: a) a first vial (1110) having a first end (1111) and an inner cavity (1113) for housing a sample, wherein the first end (1111) of the first vial (1110) comprises or is fluidly connected to a first vial port (1114) and a second vial port (1115); b) a binding chamber (1120) having an inner cavity (1123) therein; wherein a binding component (1170) capable of binding nucleic acid is disposed in the inner cavity (1123); wherein the binding chamber (1120) is fluidly connected to a first binding chamber port (1124), a second binding chamber port (1125), a third binding chamber port (1126) and a fourth binding chamber port (1127); c) a first channel (1161) fluidly connected to the second binding chamber port (1125) and the second vial port (1115); d) a second channel (1162) fluidly connected to the first binding chamber port (1124); e) a third channel (1163) fluidly connected to the first vial port (1114); f) a first actuator (1151), wherein the first actuator (1151) fluidly connects the second channel (1162) to the third channel (1163), wherein when activated, the first actuator (1151) provides pressure for moving fluid, air, or both from the first vial (1110) to the binding chamber (1120) via the first channel (1161) and from the binding chamber (1120) to the sample vial (1110) via the second channel (1162) and third channel (1163); g) a second vial (1130) having a second end (1132) and an inner cavity (1133) for housing a fluid, wherein the second end (1132) of the second vial (1130) comprises or is fluidly connected to a first vial port (1134) and a second vial port (1135); h) a fourth channel (1164) fluidly connected to the first vial port (1134); i) a fifth channel (1165) fluidly connected to the third binding chamber port (1126); j) a sixth channel (1165) fluidly connected to the fourth binding chamber port (1127) and the second vial port (1135); and k) a second actuator (1152), wherein the second actuator (1152) fluidly connects the fourth channel (1164) to the fifth channel (1165), wherein when activated, the second actuator (1152) provides pressure for moving fluid, air, or both from the second vial (1130) to the binding chamber (1120) via the fourth channel (1164) and fifth channel (1165) and from the binding chamber (1120) to the second vial (1130) via the sixth channel (1166).

Embodiment 109A: A device (1100) for isolating nucleic acids from a sample, the device (1100) comprising: a) a binding chamber (1120) comprising a binding component (1170) capable of binding nucleic acid disposed therein; b) a first actuator (1151) fluidly connected to the binding chamber (1120); c) a first coupler (1140a) comprising an inner side (1142) and an outer side (1141), wherein the first coupler (1140a) is fluidly connected the binding chamber (1120) and fluidly connected to the first actuator (1151); d) a second actuator (1152) fluidly connected to the binding chamber (1120); and e) a second coupler (1140b) comprising an inner side (1142) and an outer side (1141), wherein the second coupler (1142) is fluidly connected to the binding chamber (1120) and fluidly connected to the second actuator (1152).

Embodiment 110A: The device (1100) of embodiment 109A, wherein a first end (1121) of the binding chamber (1120) comprises or is fluidly connected to a third binding chamber port (1126) and a fourth binding chamber port (1127), and a second end (1122) of the binding chamber (1120) comprises or is fluidly connected to a first binding chamber port (1124) and a second binding chamber port (1125). Embodiment 111A: The device (1100) of embodiment 109A and embodiment 110A, wherein the inner side (1142) of the first coupler (1140a) comprises or is fluidly connected to a first coupler port (1144) and a second coupler port (1145). Embodiment 112A: The device (1100) of any one of embodiments 109A-111A, wherein the inner side (1142) of the second coupler (1140b) comprises or is fluidly connected to a third coupler port (1146) and a fourth coupler port (1147). Embodiment 113A: The device (1100) of any one of embodiments 109A-111A, wherein the first actuator (1151), the first coupler (1140a) and the binding chamber (1120), and the second actuator (1152), the second coupler (1140b) and the binding chamber (1120) are fluidly connected via a plurality of channels. Embodiment 114A: The device (1100) of embodiment 113A, wherein a first channel (1161) fluidly connects the second coupler port (1145) to the second binding chamber port (1125), a second channel (1162) fluidly connects the first binding chamber port (1124) to the first actuator (1151), and a third channel (1163) fluidly connects the first coupler port (1144) to the first actuator (1151). Embodiment 115A: The device (1100) of embodiment 113A, wherein a fourth channel (1164) fluidly connects the third coupler port (1146) to the second actuator (1152), a fifth channel (1165) fluidly connects the third binding chamber port (1126) to the second actuator (1152), and a sixth channel (1166) fluidly connects the fourth binding chamber port (1127) to the fourth coupler port (1147).

Embodiment 116A: The device (1100) of any of the embodiments herein, wherein the binding component (1170) optionally subdivides the inner cavity (1233) into at least a first subcavity and a second subcavity. Embodiment 117A: The device (1100) of any of the embodiments herein, wherein the binding component (1170) is optionally immobilized in the binding chamber (1120). Embodiment 118A: The device (1100) of any of the embodiments herein, wherein the binding chamber (1120) comprises a cap, the cap can move between at least an open position to provide access to the inner cavity (1123) of the binding chamber (1120) or the binding component (1170) or both, and a closed position wherein the inner cavity (1123) of the binding chamber (1120) and binding component (1170) are not accessible. Embodiment 119A: The device (1100) of embodiment 118A, wherein the binding component (1170) is attached to the cap. Embodiment 120A: The device (1100) of any of the embodiments herein, wherein the first actuator (1151) optionally bisects a channel into a first half and a second half. Embodiment 121A: The device (1100) of embodiment 120A, wherein the first half is the second channel (1162) and the second half is the third channel (1163). Embodiment 122A: The device (1100) of any of the embodiments herein, wherein the second actuator (1152) optionally bisects a channel into a first half and a second half. Embodiment 123A: The device (1100) of embodiment 122A, wherein the first half is the fourth channel 1164) and the second half is the fifth channel (1165). Embodiment 124A: The device (1100) of any of the embodiments herein, wherein the first actuator (1151), second actuator (1152), or both are external to the channel. Embodiment 125A: The device (1100) of embodiment 124A, wherein the first actuator (1151), second actuator (1152), or both are a peristaltic pump. Embodiment 126A: The device (1100) of any of the embodiments herein, wherein the first actuator (1151), second actuator (1152), or both comprises a directional guide component. Embodiment 127A: The device (1100) of embodiment 126A, wherein the directional guide component is a valve. Embodiment 128A: The device (1100) of embodiment 127A, wherein the valve is a one-way valve. Embodiment 129A: The device (1100) of any one of embodiments 126A-128A, wherein the directional guide component is a component that is adapted to allow gravity to guide fluid flow.

Embodiment 130A: The device (1100) of any of the embodiments herein, wherein the channels extend outwardly from the binding chamber (1120) and are arranged radially around the binding chamber (1120). Embodiment 131A: The device (1100) of any of the embodiments herein, wherein the channels are arranged in a configuration that occupies one or a combination of the x-axis, y-axis, and z-axis with respect to the binding chamber (1120). Embodiment 132A: The device (1100) of any of the embodiments herein, wherein the channels extend outwardly from the binding chamber (1120) in any particular configuration. Embodiment 133A: The device (1100) of any of the embodiments herein, wherein the channels are arranged radially around the binding chamber (1120) and evenly spaced. Embodiment 134A: The device of (1100) any of the embodiments herein, wherein the device (1100) is configured such that fluid in the binding chamber (1120) necessarily contacts the binding component (1170).

Embodiment 135A: A method of isolating nucleic acid from a sample, said method comprising: using a device (1100) comprising: a) conveying a first fluid through a first channel (1161) into a binding chamber (1120), the binding chamber (1120) comprising a binding component (1170) capable of binding nucleic acids disposed therein, wherein the first fluid contacts the binding component (1170); b) conveying the first fluid from the binding chamber (1120) through a second channel (1162) to or towards a first actuator (351), to effectuate the removal of the first fluid from the binding chamber (1120); c) conveying a second fluid through a fifth channel (1165) to the binding chamber (1120), wherein the second fluid contacts the binding component (1170); and d) conveying the second fluid from the binding chamber (1120) through a sixth channel (1166), to effectuate the removal of the second fluid from the binding chamber (1120).

Embodiment 136A: The method of embodiment 135A, wherein step a) comprises conveying the first fluid from a first vial (1110) through the first channel (1161) to the binding chamber (1120). Embodiment 137A: The method of embodiment 135A, wherein step b) further comprises conveying the first fluid from the first actuator (1151) through a third channel (1163). Embodiment 138A: The method of embodiment 137A, wherein step b) further comprises conveying the first fluid from the first actuator (1151) through a third channel (1163) into a first vial (1110). Embodiment 139A: The method of embodiment 135A, wherein step c) further comprises conveying the second fluid from a second actuator (1152) through the fifth channel (1165). Embodiment 140A: The method of embodiment 139A, wherein step c) comprises conveying the second fluid through a fourth channel (1164) to the second actuator (1152) and further conveying the second fluid from the second actuator (1152) through the fifth channel (1165) to the binding chamber (1120). Embodiment 141A: The method of embodiment 139A, wherein step c) comprises conveying the second fluid from a second vial (1130) through a fourth channel (1164) to a second actuator (1152) and further conveying the second fluid from the second actuator (1152) through the fifth channel (1165) to the binding chamber (320). Embodiment 142A: The method of embodiment 134A, wherein step d) comprises conveying the second fluid from the binding chamber (1120) through the sixth channel (1166) to a second vial (1130). Embodiment 143A: The method of embodiment 135A, wherein the first actuator (1151) functions as a waste chamber.

Embodiment 144A: A method of isolating nucleic acid from a sample, said method comprising: using a device (1100) comprising: a) conveying a first fluid through a first channel (1161) into a binding chamber (1120), the binding chamber (1120) comprising a binding component (1170) disposed therein, wherein the first fluid contacts the binding component (1170); b) conveying the first fluid from the binding chamber (1120) through a second channel (1162) to or towards a first actuator (1151) to effectuate the removal of the first fluid from the binding chamber (1120); c) conveying a second fluid through a fifth channel (1165) to the binding chamber (1120), wherein the second fluid contacts the binding component (1170); and d) conveying the second fluid from the binding chamber (1120) through the fifth channel (365), to effectuate the removal of the second fluid from the binding chamber (320).

### Embodiment Set B

Embodiment 1B: A device (1100) for isolating nucleic acids from a sample, the device (1100) comprising: a) a housing comprising a binding chamber (1120), a first chamber (1210), and a second chamber (1220), wherein the binding chamber (1120) comprises a binding component capable (1170) of binding nucleic acid therein; b) a first channel (1161) fluidly connecting the binding chamber (1120) and the first chamber (1210); c) a means for conveying a first fluid through the first channel (1161) between the first chamber (1210) and the binding chamber (1120); d) a second channel (1162) fluidly connecting the binding chamber (1120) and the second chamber (1220); and a means for conveying a second fluid through the second channel (1162) between the second chamber (1220) and the binding chamber (1120).

Embodiment 2B: The device (1100) of embodiment 1B, wherein the means for conveying a first fluid through the first channel (1161) comprises a first actuator (1151) operatively connected to the first chamber (1210). Embodiment 3B: The device (1100) of embodiment 1B or embodiment 2B, wherein the means for conveying a second fluid through the second channel (1162) comprises a second actuator (1152) operatively connected to the second chamber (1220). Embodiment 4B: The device (1100) of any one of embodiments 1B-3B, wherein the means for conveying a first fluid through the first channel (1161) comprises a flexible vial operatively connected to the first chamber (1210). Embodiment 5B: The device (1100) of any one of embodiments 1B-4B, wherein the means for conveying the second fluid through the second channel (1162) comprises a flexible vial operatively connected to the second chamber (1220).

Embodiment 6B: A device (1100) for isolating nucleic acids from a sample, the device (1100) comprising: a) a housing comprising a binding chamber (1120), a first chamber (1210), and a second chamber (1220), wherein the binding chamber (1120) comprises a binding component capable (1170) of binding nucleic acid; b) a first channel (1161) fluidly connecting the binding chamber (1120) and the first chamber (1210); c) a second channel (1162) fluidly connecting the binding chamber (1120) and the second chamber (1220); d) a first actuator (1151) operatively connected to the first chamber (1210) and configured to affect fluid flow through the first channel (1161) between the first chamber (1210) and the binding chamber (1120); and e) a second actuator (1152) operatively connected to the second chamber (1220) and configured to affect fluid flow through the second channel (1162) between the second chamber (1220) and the binding chamber (1120).

Embodiment 7B: The device (1100) of any one of embodiments 1B-6B, wherein the first channel (1161) is disposed on a first side (1121) of the binding chamber and a first side (1211) of the first chamber (1210); and the second channel (1162) is disposed on a second side (1122) of the binding chamber and a first side (1221) of the second chamber (1220). Embodiment 8B: The device (1100) of any one of embodiments 1B-7B, wherein the first actuator (1151) and the second actuator (1152) is a pressure pump. Embodiment 9B: The device (1100) of any one of embodiments 1B-5B, wherein the first actuator (1151) is disposed at or near a second side (1212) of the first chamber (1210). Embodiment 10B: The device (1100) of any one of embodiments 1B-9B, wherein the second actuator (1152) is disposed at or near a second side (1212) of the second chamber (1220).

Embodiment 11B: The device (1100) of any one of embodiments 1B-10B, wherein the housing further comprises a first coupler (1140a) fluidly connected to the first chamber (1210), wherein the first coupler (1140a) comprises an opening to the first chamber (1210) and is configured to engage at least a cap (1149) or a first vial (1120). Embodiment 12B: The device (1100) of any one of embodiments 1B-10B, wherein the housing further comprises a first coupler (1140a) fluidly connected to the first chamber (1210), wherein the first coupler (1140a) comprises an opening to the first chamber (1210) and is configured to engage at least a means for preventing access to the first chamber (1210) through the first coupler (1140a), e.g., a closing means. Embodiment 12.1B: The device (1100) of any one of embodiments 1B-10B, wherein the housing further comprises a first coupler fluidly connected to the first chamber, wherein the first coupler is configured to allow and/or prevent access to the first chamber. Embodiment 13B: The device (1100) of embodiment 10B, wherein the means for preventing access to the first chamber (1210) through the first coupler (1140a), e.g., a closing means, comprises a cap (1149), a seal, a plug, or a vial.

Embodiment 14B: The device (1100) of any one of embodiments 1B-13B, wherein the housing further comprises a second coupler (1140b) fluidly connected to the second chamber (1220), wherein the second coupler (1140b) comprises an opening to the second chamber (1210) and is configured to engage at least a cap (1149) or a second vial (1130). Embodiment 15B: The device (1100) of any one of embodiments 1B-13B, wherein the housing further comprises a second coupler (1140b) fluidly connected to the second chamber (1220), wherein the second coupler (1140b) comprises an opening to the second chamber (1210) and is configured to engage at least a means for preventing access to the second chamber (1220) through the second coupler (1140b), e.g., a closing means. Embodiment 15.1B: The device (1100) of any one of embodiments 1B-13B, wherein the housing further comprises a second coupler fluidly connected to the second chamber, wherein the first coupler is configured to allow and/or prevent access to the first chamber. Embodiment 16B: The device (1100) of embodiment 15B, wherein the means for preventing access to the second chamber (1220) through the second coupler (1140b), e.g., a closing means, comprises a cap (1149), a seal, a plug, or a vial.

Embodiment 17B: The device (1100) of any one of embodiments 1B-16B, wherein the binding component (1170) comprises cellulose. Embodiment 18B: The device (1100) of any one of embodiments 1B-17B, wherein the binding component (1170) is immobilized in the binding chamber (1120).

Embodiment 19B: The device (1100) of any one of embodiments 1B-18B, wherein the sample is a biological sample. Embodiment 20B: The device (1100) of any one of embodiments 1B-18B, wherein the sample is an environmental sample.

Embodiment 21B: A device (1100) for isolating nucleic acids from a sample, the device (1100) comprising: a) a housing comprising a binding chamber (1120), the binding chamber (1120) comprising a binding component (1170) therein capable of binding nucleic acid; b) a first channel (1161) fluidly connected to the binding chamber (1120); c) a second channel (1162) fluidly connected to the binding chamber (1120); d) a first actuator (1151) operatively connected to the first channel (1161) and configured to affect fluid flow through the first channel (1161) between a first chamber (1210) and the binding chamber (1120); and e) a second actuator (1152) operatively connected to the second channel (1162) and configured to affect fluid flow through the second channel (1162) between a second chamber (1220) and the binding chamber (1120).

Embodiment 22B: The device (1100) of embodiment 21B, wherein the first chamber (1210) is either within the housing or external to the housing. Embodiment 23B: The device (1100) of embodiment 21B or embodiment 22B, wherein the second chamber (1220) is either within the housing or external to the housing. Embodiment 24B: The device (1100) of any one of embodiments 21B-23B, wherein the first channel (1161) is disposed on a first side (1121) of the binding chamber; and the second channel (1162) is disposed on a second side (1122) of the binding chamber. Embodiment 25B: The device (1100) of any one of embodiments 21B-24B, wherein the first actuator (1151) and the second actuator (1152) is a pressure pump. Embodiment 26B: The device (1100) of any one of embodiments 21B-25B, wherein the binding component (1170) comprises cellulose.

Embodiment 27B: The device (1100) of any one of embodiments 21B-26B, wherein the binding component (1170) is immobilized in the binding chamber (1120). Embodiment 28B: The device (1100) of any one of embodiments 21B-27B, wherein the sample is a biological sample. Embodiment 29B: The device (1100) of any one of embodiments 21B-27B, wherein the sample is an environmental sample.

Embodiment 30B: A device (1100) for isolating nucleic acids from a sample, the device (1100) comprising: a) a binding chamber (1120) comprising a binding component (1170) capable of binding nucleic acid disposed therein; b) a first chamber (1210) fluidly connected to the binding chamber (1120); c) a first actuator (1151) fluidly connected to the first chamber (1120); d) a second chamber (1220) fluidly connected to the binding chamber (1120); and e) a second actuator (1152) fluidly connected to the second chamber (1220). Embodiment 31B: The device (1100) of embodiment 30B, wherein the first chamber (1210) and the binding chamber (1120) are fluidly connected via a first channel (1161). Embodiment 32B: The device (1100) of embodiment 31B, wherein the first channel (1161) is disposed on a first side (1121) of the binding chamber and a first side (1211) of the first chamber (1210). Embodiment 33B: The device (1100) of any one of embodiments 30B-32B, wherein the second chamber (1220) and the binding chamber (1120) are fluidly connected via a second channel (1162). Embodiment 34B: The device (1100) of embodiment 33B, wherein the second channel (1162) is disposed on a second side (1122) of the binding chamber and a first side (1221) of the second chamber (1220). Embodiment 35B: The device (1100) of any one of embodiments 30B-34B, wherein the first actuator (1151) and the second actuator (1152) is a pressure pump. Embodiment 36B: The device (1100) of any one of embodiments 30B-35B, wherein the first actuator (1151) is disposed at or near a second side (1212) of the first chamber (1210). Embodiment 37B: The device (1100) of any one of embodiments 30B-36B, wherein the second actuator (1152) is disposed at or near a second side (1212) of the second chamber (1220). Embodiment 38B: The device (1100) of any one of embodiments 30B-37B, wherein the first chamber (1210), the binding chamber (1120), and the second chamber (1220) are manufactured from a single molded piece. Embodiment 39B: The device (1100) of any one of embodiments 30B-37B, wherein the first chamber (1210), the binding chamber (1120), and the second chamber (1220) are disposed within a housing.

Embodiment 40B: The device (1100) of any one of embodiments 30B-39B further comprises a first coupler (1140a) fluidly connected to the first chamber (1210), wherein the first coupler (1140a) comprises an opening to the first chamber (1210) and is configured to engage at least a cap (1149) or a first vial (1120). Embodiment 41B: The device (1100) of any one of embodiments 30B-39B further comprises a first coupler (1140a) fluidly connected to the first chamber (1210), wherein the first coupler (1140a) comprises an opening to the first chamber (1210) and is configured to engage at least a means for preventing access to the first chamber (1210) through the first coupler (1140a), e.g., a closing means. Embodiment 42B: The device (1100) of embodiment 41B, wherein the means for preventing access to the first chamber (1210) through the first coupler (1140a), e.g., a closing means, comprises a cap (1149), a seal, a plug, or a vial. Embodiment 43B: The device (1100) of any one of embodiments 30B-42B further comprises a second coupler (1140b) fluidly connected to the second chamber (1220), wherein the second coupler (1140b) comprises an opening to the second chamber (1210) and is configured to engage at least a cap (1149) or a second vial (1130). Embodiment 44B: The device (1100) of any one of embodiments 30B-44 further comprises a second coupler (1140b) fluidly connected to the second chamber (1220), wherein the second coupler (1140b) comprises an opening to the second chamber (1210) and is configured to engage at least a means for preventing access to the second chamber (1220) through the second coupler (1140b), e.g., a closing means. Embodiment 45B: The device (1100) of embodiment 44B, wherein the means for preventing access to the second chamber (1220) through the second coupler (1140b), e.g., a closing means, comprises a cap (1149), a seal, a plug, or a vial.

Embodiment 46B: The device (1100) of any one of embodiments 30B-45B, wherein the binding component (1170) comprises cellulose. Embodiment 47B: The device (1100) of any one of embodiments 30B-46B, wherein the binding component (1170) is immobilized in the binding chamber (1120). Embodiment 48B: The device (1100) of any one of embodiments 30B-47B, wherein the sample is a biological sample. Embodiment 49B: The device (1100) of any one of embodiments 28B-47B, wherein the sample is an environmental sample.

Embodiment 50B: A device (1100) for isolating nucleic acid from a sample, said device (1100) comprising: a) a binding chamber (1120) having an inner cavity therein (1123), wherein a binding component (1170) capable of binding nucleic acid is disposed in the inner cavity (1123); b) a first channel (1161) having a first end fluidly connected to the binding chamber (1120); c) a first chamber (1210) fluidly connected a second end of the first channel (1161); d) a means for conveying a first fluid between the first chamber (1210) and the binding chamber (1120) through the first channel (1161); e) a second channel (1162) having a first end fluidly connected to the binding chamber (1120); f) a second chamber (1220) fluidly connected a second end of the second channel (1162); and g) a means for conveying a second fluid between the second chamber (1220) and the binding chamber (1120) through the second channel (1162).

Embodiment 51B: The device (1100) of embodiment 50B, wherein the means for conveying a first fluid through the first channel (1161) comprises a first actuator (1151) operatively connected to the first chamber (1210). Embodiment 52B: The device (1100) of embodiment 50B or embodiment 51B, wherein the means for conveying a second fluid through the second channel (1162) comprises a second actuator (1152) operatively connected to the second chamber (1220). Embodiment 53B: The device (1100) of any one of embodiments 50B-52B, wherein the means for conveying a first fluid through the first channel (1161) comprises a flexible vial operatively connected to the first chamber (1210). Embodiment 54B: The device (1100) of any one of embodiments 50B-53B, wherein the means for conveying the second fluid through the second channel (1162) comprises a flexible vial operatively connected to the second chamber (1220).

Embodiment 55B: A device (1100) for isolating nucleic acid from a sample, said device (1100) comprising: a) a binding chamber (1120) having an inner cavity therein (1123), wherein a binding component (1170) capable of binding nucleic acid is disposed in the inner cavity (1123); b) a first channel (1161) having a first end fluidly connected to the binding chamber (1120); c) a first chamber (1210) fluidly connected a second end of the first channel (1161); d) a first actuator (1151) operatively connected to the first chamber (1210), the first actuator (1151) is configured to assist movement of a fluid through the first channel (1161) between the binding chamber (1120) and the first chamber (1210); e) a second channel (1162) having a first end fluidly connected to the binding chamber (1120); f) a second chamber (1220) fluidly connected a second end of the second channel (1162); and g) a second actuator (1152) operatively connected to the second chamber (1220), the second actuator (1152) is configured to assist movement of a fluid through the second channel (1162) between the binding chamber (1120) and the second chamber (1220).

Embodiment 56B: The device (1100) of any one of embodiments 50B-55B, wherein the first end of the first channel (1161) is disposed on a first side (1121) of the binding chamber and a second side end of the first channel (1162) is disposed on a first side (1211) of the first chamber (1210). Embodiment 57B: The device (1100) of any one of embodiments 50B-56B, wherein the first end of the second channel (1162) is disposed on a second side (1122) of the binding chamber and the second end of the second channel (1162) is disposed on a first side (1221) of the second chamber (1220). Embodiment 58B: The device (1100) of any one of embodiments 50B-57B, wherein the first actuator (1151) and the second actuator (1152) is a pressure pump. Embodiment 59B: The device (1100) of any one of embodiments 50B-58B, wherein the first actuator (1151) is disposed at or near a second side (1212) of the first chamber (1210). Embodiment 60B: The device (1100) of any one of embodiments 50B-59B, wherein the second actuator (1152) is disposed at or near a second side (1212) of the second chamber (1220). Embodiment 61B: The device (1100) of any one of embodiments 50B-60B, wherein the first chamber (1210), the first channel (1161), the binding chamber (1120), the second channel (1162), and the second chamber (1220) are manufactured from a single molded piece. Embodiment 62B: The device (1100) of any one of embodiments 50B-60B, wherein the first chamber (1210), the first channel (1161), the binding chamber (1120), the second channel (1162), and the second chamber (1220) are disposed within a housing.

Embodiment 63B: The device (1100) of any one of embodiments 50B-62B further comprises a first coupler (1140a) fluidly connected to the first chamber (1210), wherein the first coupler (1140a) comprises an opening to the first chamber (1210) and is configured to engage at least a cap (1149) or a first vial (1120). Embodiment 64B: The device (1100) of any one of embodiments 50B-62B further comprises a first coupler (1140a) fluidly connected to the first chamber (1210), wherein the first coupler (1140a) comprises an opening to the first chamber (1210) and is configured to engage at least a means for preventing access to the first chamber (1210) through the first coupler (1140a), e.g., a closing means. Embodiment 65B: The device (1100) of embodiment 60B, wherein the means for preventing access to the first chamber (1210) through the first coupler (1140a), e.g., a closing means, comprises a cap (1149), a seal, a plug, or a vial. Embodiment 66B: The device (1100) of any one of embodiments 50B-65B further comprises a second coupler (1140b) fluidly connected to the second chamber (1220), wherein the second coupler (1140b) comprises an opening to the second chamber (1210) and is configured to engage at least a cap (1149) or a second vial (1130). Embodiment 67B: The device (1100) of any one of embodiments 50B-65B further comprises a second coupler (1140b) fluidly connected to the second chamber (1220), wherein the second coupler (1140b) comprises an opening to the second chamber (1210) and is configured to engage at least a means for preventing access to the second chamber (1220) through the second coupler (1140b), e.g., a closing means. Embodiment 68B: The device (1100) of embodiment 67B, wherein the means for preventing access to the second chamber (1220) through the second coupler (1140b), e.g., a closing means, comprises a cap (1149), a seal, a plug, or a vial.

Embodiment 69B: The device (1100) of any one of embodiments 50B-68B, wherein the binding component (1170) comprises cellulose. Embodiment 70B: The device (1100) of any one of embodiments 50B-69B, wherein the binding component (1170) is immobilized in the binding chamber (1120). Embodiment 71B: The device (1100) of any one of embodiments 50B-70B, wherein the sample is a biological sample. Embodiment 72B: The device (1100) of any one of embodiments 50B-70B, wherein the sample is an environmental sample.

Embodiment 73: A device (1100) for isolating nucleic acid from a sample, said device (1100) comprising: a) a binding chamber (1120) having an inner cavity therein (1123), wherein a binding component (1170) capable of binding nucleic acid is disposed in the inner cavity (1123); b) a first channel (1161) having a first end fluidly connected to the binding chamber (1120); c) a first chamber (1210) fluidly connected a second end of the first channel (1161); d) a first coupler (1140a) fluidly connected to the first chamber (1210), wherein the first coupler (1140a) comprises an opening to the first chamber (1210) and is configured to engage at least a means for preventing access to the first chamber (1210) through the first coupler (1140a), e.g., a closing means; e) a means for conveying a first fluid between the first chamber (1210) and the binding chamber (1120) through the first channel (1161); f) a second channel (1162) having a first end fluidly connected to the binding chamber (1120); g) a second chamber (1220) fluidly connected a second end of the second channel (1162); and h) a means for conveying a second fluid between the second chamber (1220) and the binding chamber (1120) through the second channel (1162).

Embodiment 74B: The device (1100) of embodiment 73B, wherein the means for conveying a first fluid through the first channel (1161) comprises a first actuator (1151) operatively connected to the first chamber (1210). Embodiment 75B: The device (1100) of embodiment 73B or embodiment 74B, wherein the means for conveying a second fluid through the second channel (1162) comprises a second actuator (1152) operatively connected to the second chamber (1220). Embodiment 76B: The device (1100) of any one of embodiments 73B-75B, wherein the means for conveying a first fluid through the first channel (1161) comprises a flexible vial operatively connected to the first chamber (1210). Embodiment 77B: The device (1100) of any one of embodiments 73B-76B, wherein the means for conveying the second fluid through the second channel (1162) comprises a flexible vial operatively connected to the second chamber (1220).

Embodiment 78B: A device (1100) for isolating nucleic acid from a sample, said device (1100) comprising: a) a binding chamber (1120) having an inner cavity therein (1123), wherein a binding component (1170) capable of binding nucleic acid is disposed in the inner cavity (1123); b) a first channel (1161) having a first end fluidly connected to the binding chamber (1120); c) a first chamber (1210) fluidly connected a second end of the first channel (1161); d) a first coupler (1140a) fluidly connected to the first chamber (1210), wherein the first coupler (1140a) comprises an opening to the first chamber (1210) and is configured to engage at least a means for preventing access to the first chamber (1210) through the first coupler (1140a), e.g., a closing means; e) a first actuator (1151) operatively connected to the first chamber (1210), the first actuator (1151) is configured to assist movement of a fluid through the first channel (1161) between the binding chamber (1120) and the first chamber (1210); f) a second channel (1162) having a first end fluidly connected to the binding chamber (1120); g) a second chamber (1220) fluidly connected a second end of the second channel (1162); and h) a second actuator (1152) operatively connected to the second chamber (1220), the second actuator (1152) is configured to assist movement of a fluid through the second channel (1162) between the binding chamber (1120) and the second chamber (1220).

Embodiment 79B: The device (1100) of any one of embodiments 73B-78B, wherein the first end of the first channel (1161) is disposed on a first side (1121) of the binding chamber and a second side end of the first channel (1162) is disposed on a first side (1211) of the first chamber (1210). Embodiment 80B: The device (1100) of any one of embodiments 73B-79B, wherein the first end of the second channel (1162) is disposed on a second side (1122) of the binding chamber and the second end of the second channel (1162) is disposed on a first side (1221) of the second chamber (1220). Embodiment 81B: The device (1100) of any one of embodiments 73B-80B, wherein the first actuator (1151) and the second actuator (1152) is a pressure pump. Embodiment 82B: The device (1100) of any one of embodiments 73B-81B, wherein the first actuator (1151) is disposed at or near a second side (1212) of the first chamber (1210). Embodiment 83B: The device (1100) of any one of embodiments 73B-82B, wherein the second actuator (1152) is disposed at or near a second side (1212) of the second chamber (1220). Embodiment 84B: The device (1100) of any one of embodiments 73B-83B, wherein the first chamber (1210), the first channel (1161), the binding chamber (1120), the second channel (1162), and the second chamber (1220) are manufactured from a single molded piece. Embodiment 85B: The device (1100) of any one of embodiments 73B-84B, wherein the first chamber (1210), the first channel (1161), the binding chamber (1120), the second channel (1162), and the second chamber (1220) are disposed within a housing. Embodiment 86B: The device (1100) of any one of embodiments 73B-85B, the means for preventing access to the first chamber (1210) through the first coupler (1140a), e.g., a closing means, comprises a cap (1149), a seal, a plug, or a vial. Embodiment 87B: The device (1100) of any one of embodiments 73B-85B, wherein the first coupler (1140a) is configured to engage at least a cap (1149) or a first vial (1120). Embodiment 88B: The device (1100) of any one of embodiments 73B-87B, wherein the means for preventing access to the second chamber (1220) through the second coupler (1140b), e.g., a closing means, comprises a cap (1149), a seal, a plug, or a vial. Embodiment 89B: The device (1100) of any one of embodiments 73B-87B, wherein the second coupler (1140b) is configured to engage at least a cap (1149) or a second vial (1130).

Embodiment 90B: The device (1100) of any one of embodiments 73B-89B, wherein the binding component (1170) comprises cellulose. Embodiment 91B: The device (1100) of any one of embodiments 73-90B, wherein the binding component (1170) is immobilized in the binding chamber (1120). Embodiment 92B: The device (1100) of any one of embodiments 73B-91B, wherein the sample is a biological sample. Embodiment 93B: The device (1100) any one of embodiments 73B-91B, wherein the sample is an environmental sample.

Embodiment 94B: A method of isolating nucleic acid from a sample, said method comprising: using a device (1100) comprising: a) conveying a first fluid through a first channel (1161) into a binding chamber (1120), the binding chamber (1120) comprising a binding component (1170) capable of binding nucleic acids disposed therein, wherein the first fluid contacts the binding component (370); b) conveying the first fluid from the binding chamber (1120) through the first channel (1161), to effectuate the removal of the first fluid from the binding chamber (1120); c) conveying a second fluid through a second channel (1162) to the binding chamber (1120), wherein the second fluid contacts the binding component (1170); and d) conveying the second fluid from the binding chamber (1120) through the second channel (1162), to effectuate the removal of the second fluid from the binding chamber (1120).

Embodiment 95B: The method of embodiment 94B, wherein step a) comprises conveying the first fluid from a first chamber (1210) through the first channel (1161) into the binding chamber (1120). Embodiment 96B: The method of embodiment 94B, wherein step b) comprises conveying the first fluid from the binding chamber (1120) through the first channel (1161) into the first chamber (1210). Embodiment 97B: The method of embodiment 94B, wherein step c) comprises conveying a second fluid from a second chamber (1220) through the second channel (1162) into the binding chamber (1120). Embodiment 98B: The method of embodiment 94B, wherein step d) comprises conveying the second fluid from the binding chamber (1120) through the second channel (1162) into the second chamber (1220).

Embodiment 99B: The method of any one of embodiments 94B-98B, wherein the first fluid comprises a lysis buffer, a biological sample, or a combination thereof. Embodiment 100B: The method of any one of embodiments 94B-99B, wherein the second fluid comprises an elution buffer. Embodiment 101B: The method of any one of embodiments 94B-99B, wherein the second fluid comprises a wash buffer.

Embodiment 102B: A system (100) for isolating nucleic acids from a sample, the system comprising: a) a binding chamber (120), the binding chamber comprising: i) a binding component (170) disposed in an inner cavity (123) of the binding chamber (120), the binding component (170) is capable of binding nucleic acid; ii) a first binding chamber port (124) disposed in the binding chamber (120) for providing access to the inner cavity (123) of the binding chamber (120); and b) a sample chamber (110) having an inner cavity (113) for housing a fluid sample, the sample chamber (110) comprises a sample chamber port (114) for providing access to the inner cavity (113) of the sample chamber (110), the sample chamber port (114) and the first binding chamber port (124) are adapted to engage so as to fluidly connect the inner cavity (113) of the sample chamber (110) and the inner cavity (123) of the binding chamber (120); and the sample chamber (110) either functions as an actuator or comprises an actuator component to facilitate movement of fluid from the inner cavity (113) of the sample chamber (110) to the inner cavity (123) of the binding chamber (120) or from the inner cavity (123) of the binding chamber (120) of the inner cavity (113) of the sample chamber (110).

Embodiment 103B: The system (100) of embodiment 102B, wherein the binding chamber (120) further comprises a second binding chamber port (125) disposed for providing access to the inner cavity (123) of the binding chamber. Embodiment 104B: The system (100) of embodiment 103B, wherein the first binding chamber port (124) is positioned on an opposite end of the binding chamber (120) to the second binding chamber port (125). Embodiment 105B: The system (100) of embodiment 103B, wherein the first binding chamber port (124) is disposed on a first end (121) of the binding chamber (120) and the second binding chamber port (125) is disposed on a second end (122) of the binding chamber (120); wherein the second end (122) is opposite the first end (121).

Embodiment 106B: The system (100) of any of embodiments 102B-105B, wherein the system (100) further comprises a second chamber (130) having an inner cavity (133) for housing a fluid sample, the second chamber (130) comprises a second chamber port (134) for providing access to the inner cavity (133) of the second chamber (130), the second chamber port (134) and the second binding chamber port (125) are adapted to engage so as to fluidly connect the inner cavity (133) of the second chamber (130) and the inner cavity (123) of the binding chamber (120); and the second chamber (130) either functions as an actuator or comprises an actuator component to facilitate movement of fluid from the inner cavity (133) of the second chamber (130) to the inner cavity (123) of the binding chamber (120) or from the inner cavity (123) of the binding chamber (120) of the inner cavity (133) of the second chamber (130). Embodiment 107B: The system (100) of any of embodiments 102B-106B, wherein the fluid sample is a wash buffer or elution buffer. Embodiment 108B: The system (100) of embodiment 102B, wherein the fluid sample is a test sample or a buffer. Embodiment 109B: The system (100) of embodiment 102B, wherein at least a portion of the sample chamber (110) is constructed from a flexible material to allow the sample chamber (102) to be squeezed. Embodiment 110B: The system (100) of embodiment 102B, wherein the first binding chamber port (124) is configured to move between at least an open position to allow fluid in or out of the binding chamber (120) and a closed position to prevent fluid flowing in or out of the binding chamber (120). Embodiment 111B: The system (100) of embodiment 110B, wherein the first binding chamber port (124) comprises a valve that occupies a closed position when the first sample chamber port (114) is not engaged with the first binding chamber port (124).

Embodiment 112B: The system (100) of any one of embodiments 102B-111B, wherein the binding component (127) comprises cellulose. Embodiment 113B: The system (100) of any one of embodiments 102B-112B, wherein the binding component (127) is immobilized in the binding chamber (120).

Embodiment 114B: A device (1100) for isolating nucleic acids from a sample, the device (1100) comprising: a) a housing comprising a binding chamber (1120), a first chamber (1210), and a second chamber (1220), wherein the binding chamber (1120) comprises a binding component capable (1170) of binding nucleic acid therein; b) a first channel (1161) fluidly connecting the binding chamber (1120) and the first chamber (1210); c) a second channel (1162) fluidly connecting the binding chamber (1120) and the second chamber (1220); and d) a first actuator (1151) configured to affect fluid flow through at least the first channel (1161) between the first chamber (1210) and the binding chamber (1120).

Embodiment 115B: The device (1100) of embodiment 114B, wherein the first actuator (1151) is operatively connected to the first chamber (1210). Embodiment 116B: The device (1100) of embodiment 114B or 115B, wherein the first actuator (1151) is configured to affect fluid flow through the second channel (1162) between the second chamber (1220) and the binding chamber (1120). Embodiment 117B: The device of embodiment 114B or embodiment 115B further comprising a second actuator (1152) operatively connected to the second chamber (1220). Embodiment 118B: The device of embodiment 117B, wherein the second actuator (1152) is configured to affect fluid flow through the second channel (1162) between the second chamber (1220) and the binding chamber (1120).

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

The reference numbers recited in the below claims are solely for ease of examination of this patent application, and are exemplary, and are not intended in any way to limit the scope of the claims to the particular features having the corresponding reference numbers in the drawings.

What is claimed is:

1. A device (1100) for isolating nucleic acids from a sample, the device (1100) comprising:
a) a housing comprising a binding chamber (1120), a first chamber (1210), and a second chamber (1220), wherein the binding chamber (1120) comprises a binding component capable (1170) of binding nucleic acid therein;
b) a first channel (1161) fluidly connecting the binding chamber (1120) and the first chamber (1210);
c) a means for conveying a first fluid through the first channel (1161) between the first chamber (1210) and the binding chamber (1120);
d) a second channel (1162) fluidly connecting the binding chamber (1120) and the second chamber (1220); and
e) a means for conveying a second fluid through the second channel (1162) between the second chamber (1220) and the binding chamber (1120).

2. The device (1100) of claim 1, wherein the binding component (1170) comprises cellulose.

3. The device (1100) of claim 1 or 2, wherein the means for conveying the first fluid through the first channel (1161) comprises a first actuator (1151) operatively connected to the first chamber (1210).

4. The device (1100) of any of claims 1-3, wherein the means for conveying the second fluid through the second channel (1162) comprises a second actuator (1152) operatively connected to the second chamber (1220).

5. The device (1100) of claim 1 or 2, wherein the means for conveying a first fluid through the first channel (1161) comprises a flexible vial operatively connected to the first chamber (1210).

6. The device (1100) of claim 1, 2, or 5, wherein the means for conveying the second fluid through the second channel (1162) comprises a flexible vial operatively connected to the second chamber (1220).

7. The device (1100) of claim 1, wherein the first channel (1161) is fluidly connected to a first side (1121) of the binding chamber (1120); and the second channel (1162) if fluidly connected to a second side (1122) of the binding chamber (1120).

8. A device (1100) for isolating nucleic acids from a sample, the device (1100) comprising:
a) a housing comprising a binding chamber (1120), a first chamber (1210), and a second chamber (1220), wherein the binding chamber (1120) comprises a binding component capable (1170) of binding nucleic acid;
b) a first channel (1161) fluidly connecting the binding chamber (1120) and the first chamber (1210);
c) a second channel (1162) fluidly connecting the binding chamber (1120) and the second chamber (1220);
d) a first actuator (1151) operatively connected to the first chamber (1210) and configured to affect fluid flow through the first channel (1161) between the first chamber (1210) and the binding chamber (1120); and
e) a second actuator (1152) operatively connected to the second chamber (1220) and configured to affect fluid flow through the second channel (1162) between the second chamber (1220) and the binding chamber (1120).

9. The device (1100) of any one of claims 1-8, wherein the first channel (1161) is fluidly connected to a first side (1121) of the binding chamber (1120); and the second channel (1162) if fluidly connected to a second side (1122) of the binding chamber (1120).

10. The device (1100) of any one of claim 3-4, or 8-9, wherein the first actuator (1151) and the second actuator (1152) is a pressure pump.

11. The device (1100) of any one of claim 3-4, or 8-10, wherein the first actuator (1151) is disposed at or near a second side (1212) of the first chamber (1210).

12. The device (1100) of any one of claim 3-4, or 8-11, wherein the second actuator (1152) is disposed at or near a second side (1212) of the second chamber (1220).

13. The device (1100) of any of claims 1-12, wherein the housing further comprises a first coupler (1140a) fluidly connected to the first chamber (1210), wherein the first coupler (1140a) comprises an opening to the first chamber (1210) and is configured to engage a means for preventing access to the first chamber (1210) through the first coupler (1140a).

14. The device (1100) of claim 13, wherein the means for preventing access to the first chamber (1210) through the first coupler (1140a) comprises a cap, a seal, a plug, or a vial.

15. The device (1100) of any of claims 1-14, wherein the housing further comprises a second coupler (1140b) fluidly connected to the second chamber (1220), wherein the second coupler (1140b) comprises an opening to the second chamber (1210) and is configured to engage a means for preventing access to the second chamber (1220) through the second coupler (1140b).

16. The device (1100) of claim 15, wherein the means for preventing access to the second chamber (1220) through the second coupler (1140b) comprises a cap, a seal, a plug, or a vial.

17. The device (1100) of any one of claims 1-16, wherein the binding component (1170) comprises cellulose.

18. The device (1100) of any one of claims 1-17, wherein the binding component (1170) is immobilized in the binding chamber (1120).

19. The device (1100) of any one of claims 1-18, wherein the sample is a biological sample.

20. The device (1100) of any one of claims 1-18, wherein the sample is an environmental sample.
